SEQUENCE LISTING (39 sequences)

SEQ ID NO: 1 - protein, 284 aa, Gallus gallus
MEAIKKKMQMLKLDKENAIDRAEQAEADKKQAEDRCKQLEEEQQGLQKKLKGTEDEVEKYSESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESRVRQLEEELRTMDQSLKSLIASEEEYSTKEDKYEEEIKLLGEKLKEAETRAEFAERSVAKLEKTIDDLEESLASAKEENVGIHQVLDQTLLELNNL

SEQ ID NO: 2 - protein, 154 aa, Gallus gallus
SESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESRVRQLEEELRTMDQSLKSLIASEEEY

SEQ ID NO: 3 - protein, 284 aa, Gallus gallus
MDAIKKKMQMLKLDKENALDRAEQAEADKKAAEERSKQLEDDIVQLEKQLRVTEDSRDQVLEELHKSEDSLLSAEENAAKAESEVASLNRRIQLVEEELDRAQERLATALQKLEEAEKAADESERGMKVIENRAQKDEEKMEIQEIQLKEAKHIAEEADRKYEEVARKLVILEGDLERAEERAELSESKCAELEEELKLVTNEAKSLEAQAEKYSQKEDKYEEEIKVLTDKLKEAETRAEFAERSVTKLEKSIDDLEEKVAHAKEENLNMHQMLDQTLLELNNM

SEQ ID NO: 4 - protein, 154 aa, Gallus gallus
LEELHKSEDSLLSAEENAAKAESEVASLNRRIQLVEEELDRAQERLATALQKLEEAEKAADESERGMKVIENRAQKDEEKMEIQEIQLKEAKHIAEEADRKYEEVARKLVILEGDLERAEERAELSESKCAELEEELKLVTNEAKSLEAQAEKY

SEQ ID NO: 5 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEDELYAQKLKYKAISEELDHALNDMTSI

SEQ ID NO: 6 - protein, 154 aa, Homo sapiens
SEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKY

SEQ ID NO: 7 - protein, 284 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESRARQLEEELRTMDQALKSLMASEEEYSTKEDKYEEEIKLLEEKLKEAETRAEFAERSVAKLEKTIDDLEETLASAKEENVEIHQTLDQTLLELNNL

SEQ ID NO: 8 - protein, 154 aa, Homo sapiens
SESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESRARQLEEELRTMDQALKSLMASEEEY

SEQ ID NO: 9 - protein, 284 aa, Homo sapiens
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 10 - protein, 154 aa, Homo sapiens
SEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKY

SEQ ID NO: 11 - protein, 248 aa, Homo sapiens
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 12 - protein, 154 aa, Homo sapiens
EDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKY

SEQ ID NO: 13 - protein, 284 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKYSTKEDKYEEEIKLLEEKLKEAETRAEFAERSVAKLEKTIDDLEDEVYAQKMKYKAISEELDNALNDITSL

SEQ ID NO: 14 - protein, 154 aa, Homo sapiens
SESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKY

SEQ ID NO: 15 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDRYEEEIKVLSDKLKEAETRAEFAERSVTKLEKSIDDLEEKVAHAKEENLSMHQMLDQTLLELNNM

SEQ ID NO: 16 - protein, 154 aa, Homo sapiens
SEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKY

SEQ ID NO: 17 - protein, 248 aa, Homo sapiens
MAGITTIEAVKRKIQVLQQQADDAEERAERLQREVEGERRAREQAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESRCREMDEQIRLMDQNLKCLSAAEEKYSQKEDKYEEEIKILTDKLKEAETRAEFAERSVAKLEKTIDDLEDKLKCTKEEHLCTQRMLDQTLLDLNEM

SEQ ID NO: 18 - protein, 154 aa, Homo sapiens
EERAERLQREVEGERRAREQAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESRCREMDEQIRLMDQNLKCLSAAEEKY

SEQ ID NO: 19 - protein, 227 aa, Homo sapiens
CRLRIFLRTASSEHLHERKLRETAEADVASLNRRIQLVEEELDRAQERLATVLQKLEEAEKAADESERGMKVIESRAQKDEEKMEIQEIQLKEAKHIAEDADRKYEEVARKLVIIESDLERAEERAELSEGQVRQLEEQLRIMDSDLESINAAEDKYSQKEDRYEEEIKVLSDKLKEAETRAEFAERSVTKLEKSIDDLEEKVAHAKEENLSMHQMLDQTLLELNNM

SEQ ID NO: 20 - protein, 154 aa, Homo sapiens
RIFLRTASSEHLHERKLRETAEADVASLNRRIQLVEEELDRAQERLATVLQKLEEAEKAADESERGMKVIESRAQKDEEKMEIQEIQLKEAKHIAEDADRKYEEVARKLVIIESDLERAEERAELSEGQVRQLEEQLRIMDSDLESINAAEDKY

SEQ ID NO: 21 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 22 - protein, 526 aa, Lepus americanus
ATLQCSWALTPTDCKTTKPLAEKALDLINKWRRDGYLFQLLRVADAHLDGAESATVYYLVLDVKETDCSVLSRKHWEDCDPDLTKRPSLDVIGQCKVIATRYSDEYQTLRLNDFNCTTSSVSSALANTKDSPVLFDFIEDTEPFRKSADKALEVYKSESEAYASFRVDRVERVTRVKGGERTNYYVDFSVRNCSRSHFHRHPAFGFCRADLSFDVEASNLENPEDVIISCEVFNFEEHGNISGFRPHLGKTPLGTDGSRDHHHPHKPHKFGCPPPQEGEDFSEGPPLQGGTPPLSPPFRPRCRHRPFGTNETHRFPHHRISVNIIHRPPPHGHHPHGPPPHGHHPHGPPPHGHPPHGPPPRHPPHGPPPHGHPPHGPPPHGHPPHGPPPHGHPPHGPPPHGHPPHGHGFHDHGPCDPPSHKEGPQDLHQHAMGPPPKHPGKRGPGKGHFPFHWRRIGSVYQLPPLQKGEVLPLPEANFPQLLLRNHTHPLKPEIQPFPQVASERCPEEFNGEFAQLSKFFPSTFPK

SEQ ID NO: 23 - protein, 148 aa, Homo sapiens
HPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFP

SEQ ID NO: 24 - protein, 101 aa, Lepus americanus
SVNIIHRPPPHGHHPHGPPPHGHHPHGPPPHGHPPHGPPPRHPPHGPPPHGHPPHGPPPHGHPPHGPPPHGHPPHGPPPHGHPPHGHGFHDHGPCDPPSHK

SEQ ID NO: 25 - protein, 626 aa, Homo sapiens
QESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQTDPNGLSFNPISDFPDTTSPKCPGRPWKSVSEINPTTQMKESYYFDLTDGLS

SEQ ID NO: 26 - protein, 125 aa, Homo sapiens
VSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLAS

SEQ ID NO: 27 - DNA, 28 bp, Artificial Sequence
  primer
cgggatccgtaagtccaccccacacttc

SEQ ID NO: 28 - DNA, 25 bp, Artificial Sequence
  primer
cgaattctcagcttgccaaatgctc

SEQ ID NO: 29 - protein, 5 aa, Lepus americanus
HHPHG

SEQ ID NO: 30 - protein, 5 aa, Lepus americanus
PPPHG

SEQ ID NO: 31 - protein, 5 aa, Lepus americanus
HPPHG

SEQ ID NO: 32 - protein, 5 aa, Artificial Sequence
  synthetic sequence
XXPHG

SEQ ID NO: 33 - protein, 5 aa, Artificial Sequence
  synthetic sequence
APPHG

SEQ ID NO: 34 - protein, 8 aa, Artificial Sequence
  synthetic sequence
HKNKGKKN

SEQ ID NO: 35 - protein, 8 aa, Artificial Sequence
  synthetic sequence
TRRHDWGH

SEQ ID NO: 36 - protein, 5 aa, Artificial Sequence
  synthetic sequence
AHPHG

SEQ ID NO: 37 - protein, 18 aa, Artificial Sequence
  synthetic sequence
CGPNWAGDGTYLGGGGPC

SEQ ID NO: 38 - protein, 18 aa, Artificial Sequence
  synthetic sequence
CGPNTPDPDGFWWVDGPC

SEQ ID NO: 39 - protein, 18 aa, Artificial Sequence
  synthetic sequence
CGPTIYKTDGGGETTGPC

(19) United States
(12) Patent Application Publication
(10) Pub. No.: US 2005/0124794 A1
(43) Pub. Date: Jun. 9, 2005

(54) Cell surface tropomyosin as a target of angiogenesis inhibition

(76) Inventors: Keith McCrae, Pepper Pike, OH (US); Fernando Donate, San Diego, CA (US); Jose Juarez, San Diego, CA (US); Andrew P. Mazar, San Diego, CA (US)

Correspondence Address: MCKENNA LONG & ALDRIDGE LLP, 1900 K STREET, NW, WASHINGTON, DC, 20006, US

(21) Appl. No.: 10/507,734

(22) PCT Filed: Mar. 17, 2003
(86) PCT No.: PCT/US03/08060

Related U.S. Application Data
(60) Provisional application No. 60/364,047, filed on Mar. 15, 2002.

(51) Int. Cl. A61K 38/17; A61K 38/12; C07K 14/705; C07K 16/30; C07K 7/64

(52) U.S. Cl. 530/350; 530/388.22; 530/317; 435/69.1; 435/325; 435/320.1; 424/144.1; 514/9; 514/12; 536/23.5

(57) ABSTRACT

The present invention is directed to novel methods for inhibiting angiogenesis and treating tumors and cancer by targeting tropomyosin (Tpm) expressed on the surface of endothelial cells and/or tumor cells, to Tpm polypeptides and peptides, as well as variants and derivatives thereof that bind inhibitors of angiogenesis, and to anti-Tpm antibodies that block or stimulate angiogenesis. Cyclic peptides that bind to the D5 subunit of HKₐ and inhibit angiogenesis are also included. Method for screening test compounds as candidate antiangiogenic molecule that binds to Tpm are disclosed, as are affinity ligands comprising the proteins, peptides, variants and derivatives of the invention.

### BACKGROUND OF THE INVENTION

1. Field of the Invention

The present invention in the field of biochemistry and medicine is directed to novel methods for inhibiting angiogenesis and treating tumors and cancer by targeting tropomyosin expressed on the surface of endothelial cells and/or tumor cells, to tropomyosin polypeptides and peptides that bind inhibitors of angiogenesis, and to anti-tropomyosin antibodies that block or stimulate angiogenesis.

2. Description of the Background Art

Angiogenesis, the formation of new capillaries form pre-existing ones (Folkman, J., N. Engl. J. Med., 1971, 285:1182-1186; Hanahan D. et al., Cell, 1996, 86:353-364), is a normal part of embryonic development, wound healing and female reproductive function. However, angiogenesis also plays a pathogenic role in the establishment and progression of certain diseases. Cancer, rheumatoid arthritis and diabetic retinopathy are examples of such diseases (Carmeliet P. et al., Nature, 2000, 407:249-257). Anti-angiogenic therapy holds promise in inhibiting the progression of these diseases.

Angiogenesis can be triggered by several pro-angiogenic cytokines. In the setting of cancer, tumor cells under hypoxic conditions secrete vascular endothelial growth factor (VEGF) and/or fibroblast growth factor (bFGF). These proteins diffuse and bind to specific receptors on endothelial cells (ECs) in the local vasculature, perturbing the balance of pro- and anti-angiogenic forces in favor of angiogenesis. As a consequence of binding these proteins, ECs are activated to (a) secrete enzymes that induce remodeling of the associated tissue matrix, and (b) change the patterns and levels of expression of adhesion molecules such as integrins. Following matrix degradation, ECs proliferate and migrate toward the hypoxic tumor, resulting in the generation and maturation of new blood vessels.

Interestingly, many anti-angiogenic factors result from the degradation of matrix proteins—i.e., are a result of the action of pro-angiogenic enzymes. Examples include endostatin, a fragment of collagen XIII (O'Reilly, M. S. et al., Cell 1997, 88:277-285); kringle 5 of plasminogen (O'Reilly, M. S. et al., Cell, 994, 79:315-328) and PEX, the C-terminus non-catalytic subunit of MMP-2 (Brooks P. C. et al., Cell, 1998, 92:391-400).

The concept has emerged that, due to the abundance of pro-angiogenic factors, these anti-angiogenic molecules are unable to overcome the pro-angiogenic balance in a primary tumor. However, since they are secreted into circulation, these anti-angiogenic molecules are capable of inhibiting angiogenesis at other locations where tumor cells may have begun to invade. Consequently, micro-metastases comprising these tumor cells at these new locations remain dormant. This hypothesis explains the puzzling observation made by surgeons many years ago: at various times after surgical removal of a primary tumor in a patient with no obvious metastatic disease, the patient returns with advanced metastatic disease.

Thus, clinical intervention by treatment with one or more of the anti-angiogenic factors could inhibit the angiogenic process and halt tumor growth as well as metastasis. Significant evidence in the literature (cited above) supports this notion.

Unregulated angiogenesis contributes to the pathology of no only many neoplastic diseases but also a number of non-neoplastic diseases associated with abnormal neovascularization including arthritis, various ocular disorders, and psoriasis. See, for example, Moses et al., 1991, Biotech. 9: 630-634; Folkman et al., 1995, N. Engl. J. Med., 333:1757-1763; Auerbach, R et al., 1985, J. Microvasc. Res. 29:401-411; Folkman, 1985, Adv Canc Res 43:175-203; Patz, A, 1982, Am. J. Opthalmol. 94:715-743; Patz, A, 1982, Am. J. Opthalmol. 94:552-554. Maintenance of the avascularity of the cornea, lens, and trabecular meshwork is crucial for vision as well as to normal ocular physiology. A number of ocular diseases, some of which lead to blindness, result from ocular neovascularization and include diabetic retinopathy, neovascular glaucoma, ocular inflammatory diseases and ocular tumors (e.g., retinoblastoma). Other eye diseases which are associated with neovascularization, including retrolental fibroplasia, uveitis, retinopathy of prematurity, and macular degeneration. About twenty eye diseases are associated with choroidal neovascularization and about forty with iris neovascularization (Waltman D D et al., 1978, Am. J. Ophthal. 85:704-710 and Gartner, S. et al., 1978, Surv. Ophthal. 22:291-312. Current treatments of these diseases, especially once neovascularization has occurred, are frequently inadequate to stave off blindness. Studies have suggested that vasoinhibitory factors which are present in normal ocular tissue (cornea and vitreous) are lost in the diseased states.

Tropomyosin (Tpm)

Tropomyosin (Tpm) was first discovered in skeletal muscle in 1948 (Bailey, K., Biochem. J. (1948) 43:271-279). Tpm binds the troponin complex, thereby playing a role in the modulation of Ca⁺²-regulated muscle contraction (Perry S V, J Muscle Res. Cell Motil. (2001) 22:5-49; Squire J M et al., FASEB J. (1998) 12:761-71). Tpm was later found to be an ubiquitous protein (Lin J J et al., Int. Rev. Cytol. (1997). 170:1-38) that is found in vertebrate non-muscle cells in many isoforms arising from four different genes as a consequence of alternative splicing and differential promoter regulation (Lin et al., supra). It binds actin, a component of the actin cytoskeleton that is involved in maintaining cell integrity and in the processes of motility, cytokinesis and exocytosis. As many other coiled coil proteins, Tpm's sequence comprises repetitions of a heptapeptide motif in a α-helix conformation (Squire et al., supra; Lin et al., supra). Each unit of the dimer is a rod-like molecule that intertwines with the other.

The first link between Tpm and angiogenesis came from a study testing the hypothesis that endostatin mediates its anti-angiogenic effect by the interaction with intracellular Tpm isoform 3 (hTm3) (MacDonald N J et al., J. Biol. Chem. (2001). 276:25190-196). This study showed binding of endostatin to permeabilized human umbilical vein ECs (HUVEC) by fluorescence microscopy and further characterized the binding of endostatin to hTm3 in vitro by Surface Plasmon Resonance (SPR). A Kd of approximately 100 μM was disclosed. This is in apparent contradiction with a significant body of literature showing endostatin biological activity in the nanomolar range (O'Reilly M S et al., Cell (1997). 88:277-285; Boehm T et al., Nature (1997) 390:404-407). McDonald et al. concluded that endostatin is internalized and binds to intracellular Tpm, resulting in disruption of the actin cytoskeleton and leading to apoptosis. However, this explanation appeared unlikely to the present inventors. This reference provides no indication as to the identity of the cell-surface receptor for endostatin. The Kd of binding to immobilized hTm3, said to reflect intracellular binding, does not correspond to the measured biological activity which should properly reflect binding to a relevant receptor. Moreover, there was no suggestion that Tpm was expressed on the cell surface or could serve as a cell surface receptor for endostatin or any other ligand on any type of cell.

Although Tpm is generally known to be an intracellular protein, it has been reported to be the extracellular domain (“ECD”) of at least two chimeric oncoproteins that were created by chromosomal translocations: the Trk oncoprotein (Nakagawara A, Cancer Lett. (2001).169:107-14) and in the TPM3-ALK fusion protein (Lamant L et al., Blood (1999). 93:3088-9510). Extracellular dimeric Tpm seemed to permanently activate the tyrosine kinase domain of the fusion protein.

Kesari K V et al. (Clin. Exp. Immunol. (1999). 118:219-227) studied Tpm isoform 5 (hTM5), an intracellular protein as a putative target autoantigen in ulcerative colitis. hTM5 was found on the surface of colon epithelial cells and cells of the LS-180 colon cancer cell line, but not on small intestinal epithelial cells, in a form loosely associated with the membrane bound colon epithelial protein (CEP). (LS-180 cells spontaneously released hTM5 as well as CEP into the culture medium.) The authors concluded that hTM5 is externalized in colon but not in small intestinal epithelial cells and, based on the physical association with CEP, suggested a possible chaperone function of CEP in the transport of hTM5.

However, any physiological role for Tpm on the cell surface in the limited number of aforementioned instances remains obscure. Moreover, there are no reports in the literature of Tpm residing on the surface of ECs.

### SUMMARY OF THE INVENTION

The present invention is directed to an isolated tropomyosin (Tpm)-related anti-angiogenic receptor polypeptide or peptide which,
(a) is a fragment of a full length native Tpm protein expressed on the surface of endothelial cells or a variant of the fragment,
(b) has a molecular mass of about 17 kDa and corresponds in its sequence to, or is a variant of, an internal fragment of a native Tpm isoform, preferably a human isoform, which is a binding site for antiangiogenic polypeptide agents, and
(c) binds to the antiangiogenic polypeptide agents which bind to the native Tpm internal fragment binding site;
   wherein the peptide has between about 4 and about 40 amino acids; and the variant of the polypeptide or peptide is a conservative substitution variant of a native Tpm sequence; and the isolated anti-angiogenic receptor polypeptide, peptide or variant has substantially the same biochemical activity of binding to the antiangiogenic polypeptide agents as does the native Tpm internal fragment.

In a preferred embodiment of the above isolated polypeptide, peptide or variant, the native Tpm isoform has an amino acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, and SEQ ID NO:19.

In yet another preferred isolated polypeptide, peptide or variant, the internal fragment of the native Tpm has an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, and SEQ ID NO:20.

The above isolated polypeptide, peptide or variant of claim is preferably one which binds to (a) human histidine-proline rich glycoprotein (HPRG); (b) rabbit HPRG; (c) a Tpm-binding, antiangiogenic homologue, variant, domain or fragment of human or rabbit HPRG; (d) two chain human kininogen human kininogen (HKₐ); (e) the D5 domain of HKₐ; or (f) a Tpm-binding, antiangiogenic homologue, variant, domain or fragment of the HKₐ or the D5 domain thereof. The above polypeptide, peptide or variant preferably binds to one or more of SEQ ID NO:21, 22, 23, 24, 25 and 26.

When the foregoing is a peptide or peptide variant, it may be capped at its N-terminus, its C-terminus, or both its N- and its C-terminus.

Also provided is a cyclic peptide which of between about 4 and about 20 amino acids which binds to the D5 domain of HKₐ and inhibit angiogenesis in an in vitro or in vivo assay of angiogenesis.

All the methods summarized herein that rely on a linear antiangiogenic peptide may be practiced with the antiangiogenic cyclic peptide of the present invention.

The invention is also directed to an antibody, preferably a monoclonal antibody (mAb), more preferably a human or humanized mAb, or an antigen-binding fragment (ABF) thereof which antibody is specific for an epitope of a Tpm isoform expressed on the surface an activated endothelial cell, which antibody or ABF has:
(a) antiangiogenic activity in that it binds to the activated endothelial cell, causing the generation of an antiangiogenic signal in the cell, resulting in (i) inhibition of migration, invasion, proliferation or angiogenesis, or (ii) apoptosis; or
(b) proangiogenic activity in that it binds, preferably by competitive binding, to Tpm on the endothelial cell and inhibits the binding to the cell of a Tpm-binding antiangiogenic agent, thereby permitting or promoting migration, invasion, proliferation or angiogenesis that would otherwise be inhibited by the antiangiogenic agent.

The above antibody or ABF preferably is specific for an epitope that is present in, or formed by, a polypeptide or peptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20.

The antibody or ABF is preferably one wherein the Tpm-binding antiangiogenic agent is a) human histidine-proline rich glycoprotein (HPRG); (b) rabbit HPRG; (c) a Tpm-binding, antiangiogenic homologue, variant, domain or fragment of human or rabbit HPRG; (d) two chain human kininogen human kininogen (HKₐ); (e) the D5 domain of HKₐ; or (f) a Tpm-binding, antiangiogenic homologue, variant, domain or fragment of the HKₐ or the D5 domain thereof.

The antibody of ABF which may be used for detecting a Tpm polypeptide or peptide that serves as an anti-angiogenic receptor on endothelial cells is preferably detectably labeled. A preferred detectable label includes a radionuclide, a PET-imageable agent, an MRI-imageable agent, a fluorescer, a fluorogen, a chromophore, a chromogen, a phosphorescer, a chemiluminescer or a bioluminescer. Preferred radionuclides include of ³H, ¹⁴C, ³⁵S, ⁶⁷Ga, ⁶⁸Ga, ⁷²As, ⁸⁹Zr, ⁹⁷Ru, ⁹⁹Tc, ¹¹¹In, ¹²³I, ¹²⁵I, ¹³¹I, ¹⁶⁹Yb and ²⁰¹Tl. Preferred fluorescers or fluorogens include fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green and Texas Red.

Also provided is a diagnostically useful Tpm-binding antibody composition comprising the above detectably labeled antibody or ABF and a diagnostically acceptable carrier.

Another embodiment is a therapeutically useful antiangiogenic antibody or ABF that targets Tpm or an epitope thereof and inhibits angiogenesis in vitro or in vivo; such a composition comprises the above antibody or ABF to which is optionally bound, directly or indirectly, a therapeutically active moiety, such as a radionuclide, a drug or a toxin radionuclide is selected from the group consisting of ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹ Au, ²¹¹At, ²¹²Pb and ²¹⁷Bi.

A therapeutic antiangiogenic pharmaceutical composition comprises the above antibody (optionally labeled with a therapeutic label) and a pharmaceutically acceptable carrier, preferably in a form suitable for injection. The composition may be one that inhibits angiogenesis in vitro, in vivo, or both.

Also provided herein is a therapeutically useful proangiogenic antibody or ABF as above (and composition that includes a pharmaceutically acceptable carrier) which antibody targets Tpm or an epitope of Tpm and stimulates angiogenesis in vitro or in vivo. This therapeutic antibody or pharmaceutical composition is preferably in a form suitable for injection.

The invention also provides a method for inhibiting endothelial cell migration, invasion, proliferation or angiogenesis, or for inducing endothelial cell apoptosis, comprising contacting endothelial cells with an effective amount of a antiangiogenic polypeptide or peptide that binds to Tpm expressed on the surface of activated endothelial cells, and thereby causes the inhibition or the apoptosis.

In this method, the Tpm-binding polypeptide may be (a) human histidine-proline rich glycoprotein (HPRG); (b) rabbit HPRG; (c) a Tpm-binding, antiangiogenic homologue, variant, domain or fragment of human or rabbit HPRG; (d) two chain human kininogen human kininogen (HKₐ); (e) the D5 domain of HKₐ; or (f) a Tpm-binding, antiangiogenic homologue, variant, domain or fragment of the HKₐ (or the D5 domain thereof; (g) troponin T; (h) tropomodulin; (i) caldesmon; (j) actin; (k) calponin; (1) pEL98; (m) glutamic dehydrogenase; and (n) a Tpm-binding, antiangiogenic homologue, variant, domain or fragment of any of (g)-(m).

Also provided is a method for treating a subject, preferably human, having a disease or condition associated with undesired cell migration, invasion, proliferation, or angiogenesis, comprising administering to the subject an effective angiogenesis-inhibiting amount of the above pharmaceutical composition. When the disease is one in which the subject has a tumor, the angiogenesis inhibition results in reduction in size or growth rate of the tumor or destruction of the tumor.

Another embodiment is a method for stimulating angiogenesis in a subject in need of enhanced angiogenesis, comprising administering to the subject an effective amount of the present pharmaceutical composition that comprises a proangiogenic antibody or ABF.

Another embodiment is a method for detecting in a biological sample the presence of Tpm of an isoform expressed on the surface of activated endothelial cells, comprising the steps of (a) contacting the sample with the present antibody or ABF in detectably labeled form, and (b) detecting the presence of the label associated with the sample.

An alternate method for detecting the presence of Tpm in a biological sample comprises
(a) contacting the sample with the detectably labeled antiangiogenic polypeptide or peptide of the invention that binds to Tpm expressed on the surface of activated endothelial cells; and
(b) detecting the presence of the label associated with the sample. Examples of such antiangiogenic polypeptides or peptides include (a) human histidine-proline rich glycoprotein (HPRG); (b) rabbit HPRG; (c) a Tpm-binding, antiangiogenic homologue, variant, domain or fragment of human or rabbit HPRG; (d) two chain human kininogen human kininogen (HKₐ); (e) the D5 domain of HKₐ; or (f) a Tpm-binding, antiangiogenic homologue, variant, domain or fragment of the HKₐ or the D5 domain thereof.

Samples which may be tested with the above methods include plasma, serum, cells, a tissue, an organ, and an extract of the cells, tissue or organ.

In the foregoing method, the contacting and the detecting may be performed in vitro; alternatively contacting is in vivo and the detecting is in vitro. The method may also be practiced wherein the contacting is in vitro and the detecting is in vivo. Finally, both contacting and detecting may be in vivo.

The present invention includes a screening test to identify a test compound as a candidate antiangiogenic molecule that binds to Tpm. Such a test comprises:
(a) adding the test compound to a mixture of a source of Tpm and a Tpm-binding antiangiogenic polypeptide or peptide agent or anti-Tpm antibody, wherein at least one of (i) the Tpm or (ii) the agent or antibody is detectably labeled
(b) in parallel, mixing similar amounts of the Tpm and the agent or antibody in the absence of the test compound; and
(c) measuring the binding of the agent with the Tpm in (a) and (b);
   wherein, if the binding in (a) is less than the binding in (b), the test is considered positive for the test compound being an inhibitor of the binding, thereby identifying the test compound as a candidate antiangiogenic molecule.

The above screening test may further comprise testing a test compound that has been identified as a candidate antiangiogenic molecule for its activity as an inhibitor of angiogenesis in an in vitro or in vivo angiogenesis assay.

Also provided herein is an affinity ligand useful for binding to or isolating a Tpm-binding antiangiogenic molecule or cells expressing the binding molecule, comprising the isolated polypeptide or peptides described above immobilized to a solid support or carrier.

The above affinity ligand provides a basis for a method for isolating a Tpm-binding antiangiogenic molecule from a complex mixture comprising:
(a) contacting the mixture with the affinity ligand;
(b) allowing any material in the mixture to bind to the ligand;
(c) removing unbound material from the ligand; and
(d) eluting the bound Tpm-binding molecule.
   The anti-angiogenic receptor polypeptide or peptide may:
(i) have the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20;
(ii) be a Tpm-binding peptide fragment of one of the above sequences; or
(iii) be a Tpm-binding conservative substitution variant of one of these sequences or of the peptide fragment thereof.

### BRIEF DESCRIPTION OF THE DRAWINGS

FIGS. 1A and 1B show inhibition of EC proliferation by HKa and prevention by an anti-Tpm antibody. FIG. 1A shows inhibition of bFGF (10 ng/ml) induced EC proliferation by 20 nM HKa (open bars) and 60 nM HKa domain 5 (black bars) is prevented by increasing concentrations of mAb TM-311. FIG. 1B shows the effect of mAb TM-311 and antibodies against other EC HK- or HKa-binding proteins on the inhibition of EC proliferation by HKa. Proliferation was measured after addition of (1) 10 ng/ml bFGF (stimulus), (2) bFGF+20 nM HKa, or (3) bFGF+HKa+antibody (300 nM). The antibodies tested were polyclonal antibodies against the urokinase receptor (Anti-uPAR) or cytokeratin 1 (Anti-CK 1), or mAbs against the receptor for the globular heads of C1q (Anti-gC1qR) or against Tpm (TM-311). Control antibodies included MOPC-21 (murine IgG1) for the mAbs and nonimmune rabbit IgG (NRIgG) for the polyclonals.

FIG. 2 shows the effect of mAb TM-311 on HKa-induced EC apoptosis, assessed by endonucleolytic cleavage of DNA. EC DNA was isolated from cells cultured for 12 hours in the presence of mAb TM-311 alone (lane 1), 20 nM HKa (lane 2), 20 nM HKa+60 nM TM-311 (lane 3), 50 nM HKa domain 5 (lane 4), 50 nM HKa domain 5+150 nM mAb TM-311 (lane 5), 2 μM 2-methoxyestradiol (lane 6) or 2 μM 2-methoxyestradiol+6 μM mAb TM-311 (lane 7).

FIG. 3 shows the results of immunoprecipitating endothelial Tpm by mAb TM-311. Cell surface proteins on proliferating and confluent HUVEC were labeled with NHS-LC biotin. Detergent extracts were prepared, and equal amounts of protein from each culture were immunoprecipitated using TM-311 or MOPC-21. Immunoprecipitated proteins were separated using 10% SDS-PAGE, transferred to PVDF, and detected using streptavidin-peroxidase and chemiluminescence.

FIGS. 4A-4C is a set of photomicrographs showing confocal laser scanning microscopic analysis of proliferating and confluent ECs. FIG. 1A shows proliferating ECs stained with control MOPC-21. FIG. 1B shows proliferating ECs stained with TM-311. FIG. 4C shows confluent ECs stained with TM-311. The image in (C) represents a compilation of 8 individual confocal “cuts” (necessary to visualize the cells), while that in (B) represents only a single cut. All cells were permeabilized by exposure to 0.1% Triton-X-100 prior to staining.

FIG. 5 shows the cross-linking of HKa to EC surface proteins. Biotin-HKa was incubated with confluent (lanes 1 and 2) or proliferating (lanes 3 and 4) ECs in the absence (lanes 1 and 3) or presence (lanes 2 and 4) of a 20-fold molar excess of unlabeled HKa prior to cross-linking using BS³. Biotin-HKa was also incubated with MDA-MB-231 breast carcinoma cells under identical conditions (lane 5). Detergent extracts were separated by SDS-PAGE, then transferred to PVDF and detected by chemiluminescence. The arrowhead denotes a prominent band of about 140-150 kDa, the expected size of an HKa-Tpm complex.

FIGS. 6A and 6B show specific binding of HKa to proliferating ECs and its inhibition by mAb TM-311. FIG. 6A shows specific binding of HKa to ECs cultured under conditions inducing proliferation. Unfixed cells were incubated with increasing concentrations of biotin-HKa in the absence or presence of 10 μM Zn²⁺. Cell-bound biotin-HKa was detected using streptavidin peroxidase and the peroxidase substrate turbo-TMB. The curve was fit by nonlinear regression, yielding a K_{d} of about 2.5 nM. FIG. 6B shows inhibition of the binding of biotin-HKa to proliferating ECs by mAb TM-311. Biotin-HKa (20 nM) was incubated with ECs in the presence of 10 μM Zn²⁺ and increasing concentrations of mAb TM-311.

FIGS. 7A-7C show specific binding of HKa to purified immobilized Tpm and its inhibition. FIG. 7A shows specific binding of HKa to purified chicken gizzard Tpm immobilized to polystyrene (in 96 well microplates). Plates were incubated with increasing concentrations of biotin-HKa in the absence or presence of 10 μM Zn²⁺. Bound ligand was measured using streptavidin-peroxidase and turbo-TMB, and the curve fit by nonlinear regression, yielding a Kd of about 2.5 nM. FIG. 7B shows Inhibition of binding of 20 nM biotin-HKa to immobilized Tpm (as above) by mAb TM-311 in the presence of 10 μM Zn²⁺. FIG. 7C shows that HKa domain 5 (D5) inhibits binding of 20 nM biotin-HKa to purified Tpm. The IC₅₀ for this inhibition was about 8.1 nM.

FIGS. 8A-8D shows the effect of HKa and/or TM-311 on in vivo angiogenesis in the chick chorioallantoic membrane (CAM). Effect of bFGF (FIG. 8A), bFGF+TM-311 (FIG. 8B), bFGF+HKa (FIG. 8C) and bFGF+HKa+TM-311 (FIG. 8D). HKa inhibited basic FGF-induced angiogenesis. The antiangiogenic effects of HKa were in turn inhibited by TM-311.

FIG. 9 shows the direct binding of an anti-Tpm antibody to immobilized Tpm. Increasing amounts of a biotinylated anti-Tpm antibody were added to a 96 well plate previously coated with 200 ng of chicken gizzard Tpm. The bound anti-Tpm antibody was detected using avidin-HRP and a chromogenic substrate. The Kd was determined by non-linear regression analysis of the empirical data.

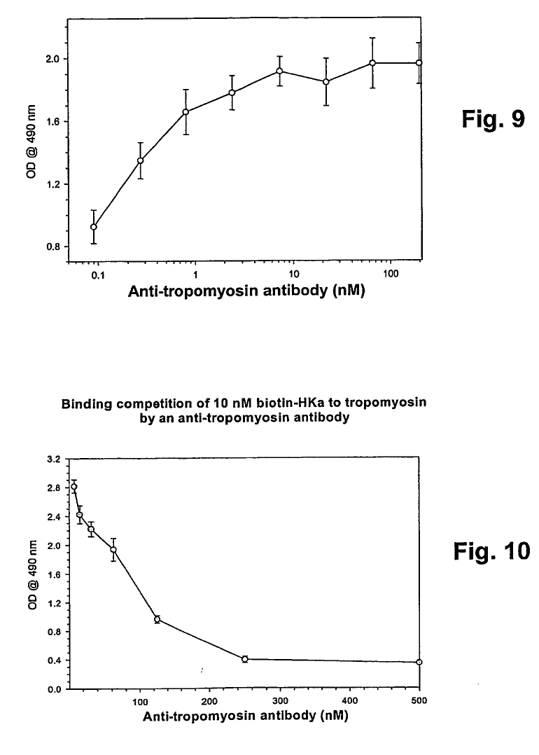

FIG. 10 shows the competition for binding of biotin-HKa to Tpm by an anti-Tpm antibody. 10 nM biotin-HKa was added to a 96 well plate previously coated with 200 ng of chicken gizzard Tpm in the presence of 10 μM ZnCl₂. Increasing amounts of anti-Tpm mAb TM311 antibody were added. Bound biotin-HKa was detected using avidin-HRP and a chromogenic substrate. Kd was determined as for FIG. 9.

FIG. 11 show the inhibition of angiogenesis by anti-Tpm mAb in the Matrigel plug model. Aliquots of Matrigel (0.5 mL) containing 400 ng/ml of bFGF, 50 μg/ml heparin with or without 20 μg of the anti-Tpm mAb or saline buffer were injected in the flanks of a mouse. After five days, the plugs were removed and the levels of hemoglobin determined. The level of hemoglobin in the positive control (no treatment) minus the negative control (no bFGF) was set as 100%.

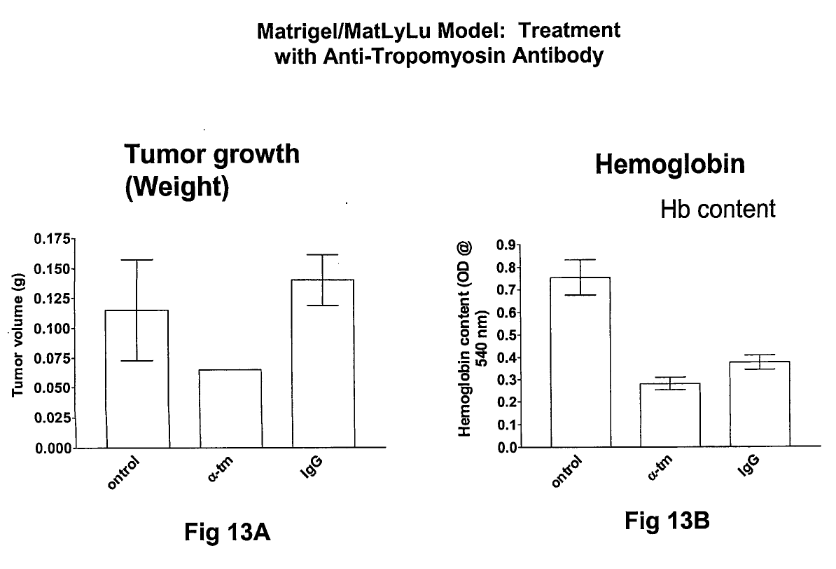

FIGS. 12 and 13 show inhibition of angiogenesis and tumor growth by anti-Tpm antibody in the Matrigel/MatLyLu model. Aliquots of Matrigel (0.5 mL) containing 2×10⁶ MatLyLu cells with or without 30 μg of the anti-Tpm mAb or saline buffer were injected in the flanks of a mouse. After seven days, the plugs were removed, scanned, weighed and the levels of hemoglobin determined. The level of hemoglobin in the positive control (no treatment) minus the negative control (no cells) was set as 100%.

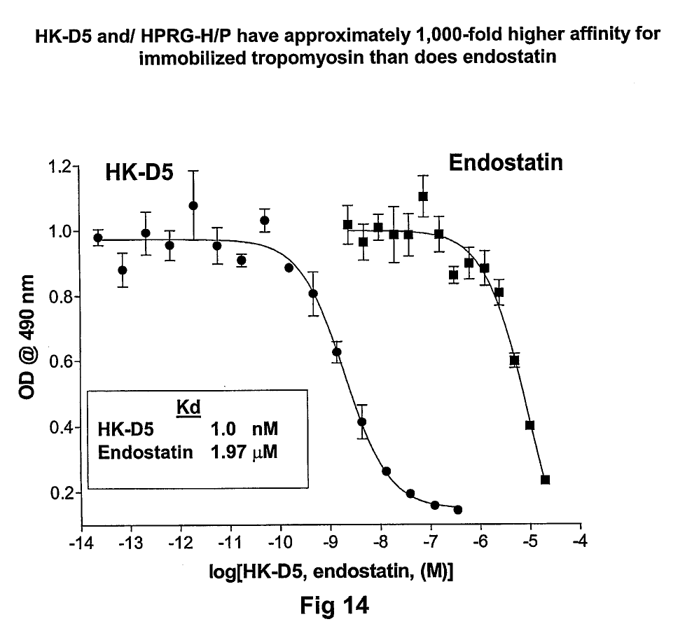

FIG. 14 shows that HK-D5 and HPRG-H/P have approximately 1,000-fold higher affinity for immobilized Tpm than does endostatin. 10 nM biotin-HKa was added to a 96 well plate previously coated with 200 ng of chicken gizzard Tpm in the presence of 10 μM ZnCl₂. Increasing amounts of HK-D5, HPRG-H/P domain or endostatin were added to the wells. Bound biotin-HKa was detected and Kd was calculated as for FIG. 10.

FIGS. 15A and 15B show that HPRG binds to immobilized chicken gizzard Tpm through its H/P domain. FIG. 15A: increasing amounts of a biotinylated HPRG, HKa or HKa-D5 were added to a 96 well Tpm-coated plate. FIG. 15B: 10 nM biotin-HKa was added to a 96 well Tpm-coated plate. Increasing amounts of HPRG-H/P domain or HPRG-N/C fragment were added to the wells. In both FIGS. 15A and 15B, bound biotin-HKa was detected and Kd was calculated as in FIG. 10.

FIG. 16 shows that ATN-228 bound to immobilized Tpm whereas ATN-246 did not. 10 nM biotin-HKa was added to a 96 well plate Tpm-coated plate. Increasing amounts of ATN-228 or ANT-246 were added to the wells. Bound biotin-HKa was detected and Kd was calculated as for FIG. 10.

FIG. 17 shows that ATN228 but not ATN246 inhibited angiogenesis in the Matrigel plug model. Aliquots of Matrigel (0.5 mL) containing 400 ng/ml of bFGF, 50 μg/ml heparin with or without 20 μg ATN-228 or ATN-246 or saline buffer were injected in the flanks of a mouse. After five days, the plugs were removed and scanned.

FIG. 18 shows that ATN230 but not ATN294 inhibited MatLyLu growth in the Matrigel model/MLL. Aliquots of Matrigel (0.5 mL) containing 2×10⁶ MatLyLu cells with or without 700 μM ATN230 or 900 μM ATN294 or saline buffer were injected in the flanks of a mouse. After seven days, the plugs were removed and weighted.

FIG. 19 shows the effects of Tpm digestion by chymotrypsin over time. Chicken gizzard Tpm (0.4 mg/ml) was incubated with 5 μg/ml chymotrypsin in TBS pH 7.5 at 37° C. and aliquots were taken at the indicated times. SDS-PAGE sample buffer was added and the sample heated at 80° C. for 4 minutes and then loaded onto and run on a 10% NuPage gel (Invitrogen).

FIG. 20 shows the results of a study identifying a fragment of Tpm that binds to HKa-D5. Chicken gizzard Tpm was partially digested with Chymotrypsin as described for FIG. 19 for 100 minutes so that a 20 kDa fragment was enriched. HK-D5 had previously immobilized in an activated Sepharose (CNBr-activated Sepharose 4 B, Amersham Biosciences). The chymotryptic fragments were incubated with the HK-D5-Sepharose resin in TBS containing 10 μM ZnCl₂, washed extensively in the same buffer and eluted in 2 M NaCl. The samples were run in a 10% NuPage gel (Invitrogen).

FIG. 21 shows that ATN-310, ATN-311 and ATN-312 displace HKa that is bound to Tpm. Ten nM biotin-HKa was added to a 96 well plate previously coated with 200 ng of chicken gizzard Tpm in the presence of 10 μM ZnCl₂. Increasing amounts of ATN-310, ATN311 or ANT-312 were added to the wells. Bound biotin-HKa was detected and Kd was calculated as in the description of FIG. 10.

### DESCRIPTION OF THE PREFERRED EMBODIMENTS

The present inventors have discovered that Tpm is present on the surface of activated ECs (ECs) and that it is an important mediator of anti-angiogenic signals.

The induction of EC apoptosis by HKa, as well as the antiangiogenic activity of HKa in the chick chorioallantoic membrane (CAM), was completely inhibited by a monoclonal anti-Tpm antibody (mAb TM-311). TM-311 also blocked the high affinity (K_{d}˜2.6 nM), Zn²⁺ -dependent binding of HKa to both purified Tpm and to proliferating ECs. Confocal microscopic analysis of ECs stained with mAb TM-311, as well as biotin labeling of cell surface proteins on intact ECs, revealed enhanced Tpm exposure on the surface of proliferating ECs. Thus, the present inventors discovered that the antiangiogenic effects of HKa are dependent upon high affinity binding to EC cell surface Tpm.

No role for Tpm as a mediator or anti-angiogenic signals had been suggested prior to the making of the present invention.

The sequence of smooth muscle Tpm (cTPM1) from chicken gizzard (ExPASy accession # P04267) is shown below as SEQ ID NO:1

| | | | | | | | |
|---|---|---|---|---|---|---|---|
| | 1 | 11 | 21 | 31 | 41 | 51 | |
| | \| | \| | \| | \| | \| | \| | |
| 1 | MEAIKKKMQM | LKLDKENAID | RAEQAEADKK | QAEDRCKQLE | EEQQGLQKKL | KGTEDEVEKY | 60 |
| 61 | SESVKEAQEK | LEQAEKKATD | AEAEVASLNR | RIQLVEEELD | RAQERLATAL | QKLEEAEKAA | 120 |
| 121 | DESERGMKVI | ENRAMKDEEK | MELQEMQLKE | AKHIAEEADR | KYEEVARKLV | VLEGELERSE | 180 |
| 181 | ERAEVAESRV | RQLEEELRTM | DQSLKSLIAS | EEEYSTKEDK | YEEEIKLLGE | KLKEAETRAE | 240 |
| 241 | FAERSVAKLE | KTIDDLEESL | ASAKEENVGI | HQVLDQTLLE | LNNL | | |

The Tyr (Y) residues after which chymotrypsin cleaved are underscored. A fragment corresponding to residues 61-214 of SEQ ID NO: 1 (underscored above) was identified as a shorter polypeptide that binds antiangiogenic polypeptide ligands such as HKa D5 (see below). This polypeptide has the following sequence and is designated SEQ ID NO:2

| | | | | | | | |
|---|---|---|---|---|---|---|---|
| 1 | SESVKEAQEK | LEQAEKKATD | AEAEVASLNR | RIQLVEEELD | RAQERLATAL | QKLEEAEKAA | 60 |
| 61 | DESERGMKVI | ENRAMKDEEK | MELQEMQLKE | AKHIAEEADR | KYEEVARKLV | VLEGELERSE | 120 |
| 121 | ERAEVAESRV | RQLEEELRTM | DQSLKSLIAS | EEEY | | | 154 |

This polypeptide is termed “Antiangiogenic Ligand-Binding Polypeptide” or “AALBP”.

The present inventors discovered the AALBP by passing a chymotryptic digest of cTPM1 over an affinity column comprising an antiangiogenic polypeptide, specifically, the D5 domain of human kininogen, HKa. Material that bound to the column was subjected to N-terminal sequencing. The sequence was determined to be SESVKEAQE, corresponding to residues 61-69 of cTPM1 (SEQ ID NO: 1) and residues 1-9 of SEQ ID NO:2. A minor contaminant had a different N terminal sequence. The size of the D5-binding polypeptide and hence its C-terminus was determined by mass spectrometry (MS), specifically, matrix-assisted laser desorption time-of-flight (MALDI-TOF) MS.

Eight human isoforms of Tpm are known.—similar—two groups—don't know which isoform it is. They are described below and their sequences shown (in an aligned format). Additional information about certain alignment comparisons between chicken and human (and chicken and chicken) Tpm is given. Also shown are the polypeptide fragments that correspond to the AALBP of cTPM1

TABLE 1

| | Tropomyosin | ExPASy | |
|---|---|---|---|
| Abbrev | species/type/isoform | Accession # | SEQ ID NO: |
| cTPM1 | chicken tropomyosin 1, | P04267 | SEQ ID NO: 1 |
| | smooth muscle (gizzard) | | |
| cTMP2 | chicken tropomyosin 2, | P04262 | SEQ ID NO: 3 |
| | smooth muscle (gizzard) | | |
| hTPM1 | human tropomyosin α chain, | P09493 | SEQ ID NO: 5 |
| | skeletal muscle | | |
| hTPM2 | human tropomyosin β chain, | P06468 | SEQ ID NO: 7 |
| | fibroblast and muscle-type | | |
| hTPM3 | human tropomyosin α chain | P06753 | SEQ ID NO: 9 |
| | skeletal, muscle type | | |
| hTPM4 | human tropomyosin, | P07226 | SEQ ID NO: 11 |
| | fibroblast non-muscle type | | |
| hTPMB | human tropomyosin β chain, | P07951 | SEQ ID NO: 13 |
| | skeletal muscle. | | |
| hTPMF | human tropomyosin α chain, | P09494 | SEQ ID NO: 15 |
| | fibroblast isoform TM3 | | |
| hTPMN | human tropomyosin, | P12324 | SEQ ID NO: 17 |
| | cytoskeletal type | | |
| hTPMS | human tropomyosin α chain, | P10469 | SEQ ID NO: 19 |
| | smooth muscle | | |

Alignment information, including % identity between certain of the full length Tpm sequences are shown in Table 2.

TABLE 2

| Alignment comparison | % Identity | # residues overlap | Score |
|---|---|---|---|
| human TPM2 and | 95.4 | 284 | 1315.0 |
| chicken gizzard TPM1 | | | |
| human TPM3 and | 77.8 | 284 | 1083.0 |
| chicken gizzard TPM1 | | | |
| chicken gizzard TPM1 and | 74.6 | 284 | 1040.0 |
| chicken gizzard TPM2 | | | |

Table 3, below shows the full length sequences of two chicken and eight human Tpm isoforms. Also indicated by underscoring is the shorter polypeptides corresponding to the AALBP as described above for cTPM1. The amino acid sequences of these shorter polypeptides appear separately in Table 4.

TABLE 3

| | | | |
|---|---|---|---|
| Abbrev | SEQUENCE | Residues | SEQ ID NO: |

| | | | | | | | |
|---|---|---|---|---|---|---|---|
| hTPM1_ | MDAIKKKMQM | LKLDKENALD | RAEQAEADKK | AAEDRSKQLE | DELVSLQKKL | 50 | SEQ ID NO:5 |
| CTPM1_ | MEAIKKKMQM | LKLDKENAID | RAEQAEADKK | QAEDRCKQLE | EEQQGLQKKL | 50 | SEQ ID NO:1 |
| hTPM2_ | MDAIKKKMQM | LKLDKENAID | RAEQAEADKK | QAEDRCKQLE | EEQQALQKKL | 50 | SEQ ID NO:7 |
| CTPM2_ | MDAIKKKMQM | LKLDKENALD | RAEQAEADKK | AAEERSKQLE | DDIVQLEKQL | 50 | SEQ ID NO:3 |
| hTPM3_ | MEAIKKKMQM | LKLDKENALD | RAEQAEAEQK | QAEERSKQLE | DELAAMQKKL | 50 | SEQ ID NO:9 |
| hTPM4_ | .......... | .......... | .......... | ......MAGL | NSLEAVKRKI | 14 | SEQ ID NO:11 |
| hTPMB_ | MDAIKKKMQM | LKLDKENAID | RAEQAEADKK | QAEDRCKQLE | EEQQALQKKL | 50 | SEQ ID NO:13 |
| hTPMF_ | MDAIKKKMQM | LKLDKENALD | RAEQAEADKK | AAEDRSKQLE | DELVSLQKKL | 50 | SEQ ID NO:15 |
| hTPMN_ | .......... | .......... | .......... | ......MAGI | TTIEAVKRKI | 14 | SEQ ID NO:17 |
| hTPM1_ | KGTEDELDKY | SEALKDAQEK | LELAEKKATD | AEADVASLNR | RIQLVEEELD | 100 | SEQ ID NO:5 |
| cTPM1_ | KGTEDEVEKY | SESVKEAQEK | LEQAEKKATD | AEAEVASLNR | RIQLVEEELD | 100 | SEQ ID NO:1 |
| hTPM2_ | KGTEDEVEKY | SESVKEAQEK | LEQAEKKATD | AEADVASLNR | RIQLVEEELD | 100 | SEQ ID NO:7 |
| cTPM2_ | RVTEDSRDQV | LEELHKSEDS | LLSAEENAAK | AESEVASLNR | RIQLVEEELD | 100 | SEQ ID NO:3 |
| hTPM3_ | KGTEDELDKY | SEALKDAQEK | LELAEKKAAD | AEAEVASLNR | RIQLVEEELD | 100 | SEQ ID NO:9 |
| hTPM4_ | QALQQQADEA | EDRAQGLQRE | LDGERERREK | AEGDVAALNR | RIQLVEEELD | 64 | SEQ ID NO:11 |
| hTPMB_ | KGTEDEVEKY | SESVKEAQEK | LEQAEKKATD | AEADVASLNR | RIQLVEEELD | 100 | SEQ ID NO:13 |
| hTPMF_ | KGTEDELDKY | SEALKDAQEK | LELAEKKATD | AEADVASLNR | RIQLVEEELD | 100 | SEQ ID NO:15 |
| hTPMN_ | QVLQQQADDA | EERAERLQRE | VEGERRAREQ | AEAEVASLNR | RIQLVEEELD | 64 | SEQ ID NO:17 |
| hTPMS_ | .......CRL | RIFLRTASSE | HLHERKLRET | AEADVASLNR | RIQLVEEELD | 43 | SEQ ID NO:19 |
| hTPM1_ | RAQERLATAL | QKLEEAEKAA | DESERGMKVI | ESRAQKDEEK | MEIQEIQLKE | 150 | SEQ ID NO:5 |
| CTPM1_ | RAQERLATAL | QKLEEAEKAA | DESERGMKVI | ENRAMKDEEK | MELQEMQLKE | 150 | SEQ ID NO:1 |
| hTPM2_ | RAQERLATAL | QKLEEAEKAA | DESERGMKVI | ENRAMKDEEK | MELQEMQLKE | 150 | SEQ ID NO:7 |
| CTPM2_ | RAQERLATAL | QKLEEAEKAA | DESERGMKVI | ENRAQKDEEK | MEIQEIQLKE | 150 | SEQ ID NO:3 |
| hTPM3_ | RAQERLATAL | QKLEEAEKAA | DESERGMKVI | ENRALKDEEK | MELQEIQLKE | 150 | SEQ ID NO:9 |
| hTPM4_ | RAGERLATAL | QKLEEAEKAA | DESERGMKVI | ENRAMKDEEK | MEIQEMQLKE | 114 | SEQ ID NO:11 |
| hTPMB_ | RAQERLATAL | QKLEEAEKAA | DESERGMKVI | ENRAMKDEEK | MELQEMQLKE | 150 | SEQ ID NO:13 |
| hTPMF_ | RAQERLATAL | QKLEEAEKAA | DESERGMKVI | ESRAQKDEEK | MEIQEIQLKE | 150 | SEQ ID NO:15 |
| hTPMN_ | RAQERLATAL | QKLEEAEKAA | DESERGMKVI | ENRALKDEEK | MELQEIQLKE | 114 | SEQ ID NO:17 |
| hTPMS_ | RAQERLATVL | QKLEEAEKAA | DESERGMKVI | ESRAQKDEEK | MEIQEIQLKE | 93 | SEQ ID NO:19 |
| HTPM1_ | AKHIAEDADR | KYEEVARKLV | IIESDLERAE | ERAELSEGKC | AELEEELKTV | 200 | SEQ ID NO:5 |
| CTPM1_ | AKHIAEEADR | KYEEVARKLV | VLEGELERSE | ERAEVAESRV | RQLEEELRTM | 200 | SEQ ID NO:1 |
| HTPM2_ | AKHIAEDSDR | KYEEVARKLV | ILEGELERSE | ERAEVAESRA | RQLEEELRTM | 200 | SEQ ID NO:7 |
| CTPM2_ | AKHIAEEADR | KYEEVARKLV | ILEGDLERAE | ERAELSESKC | AELEEELKLV | 200 | SEQ ID NO:3 |
| hTPM3_ | AKHIAEEADR | KYEEVARKLV | IIEGDLERTE | ERAELAESKC | SELEEELKNV | 200 | SEQ ID NO:9 |
| hTPM4_ | AKHIAEEADR | KYEEVARKLV | ILEGELERAE | ERAEVSELKC | GDLEEELKNV | 164 | SEQ ID NO:11 |
| hTPMB_ | AKHIAEDSDR | KYEEVARKLV | ILEGELERSE | ERAEVAESKC | GDLEEELKIV | 200 | SEQ ID NO:13 |
| hTPMF_ | AKHIAEDADR | KYEEVARKLV | IIESDLERAE | ERAELSEGQV | RQLEEQLRIM | 200 | SEQ ID NO:15 |
| hTPMN_ | AKHIAEEADR | KYEEVARKLV | IIEGDLERTE | ERAELAESRC | REMDEQIRLM | 164 | SEQ ID NO:17 |
| hTPMS_ | AKHIAEDADR | KYEEVARKLV | IIESDLERAE | ERAELSEGQV | RQLEEQLRIM | 143 | SEQ ID NO:19 |
| hTPM1_ | TNNLKSLEAQ | AEKYSQKEDR | YEEEIKVLSD | KLKEAETRAE | FAERSVTKLE | 250 | SEQ ID NO:5 |
| CTPM1_ | DQSLKSLIAS | EEEYSTKEDK | YEEEIKLLGE | KLKEAETRAE | FAERSVAKLE | 250 | SEQ ID NO:1 |
| hTPM2_ | DQALKSLMAS | EEEYSTKEDK | YEEEIKLLEE | KLKEAETRAE | FAERSVAKLE | 250 | SEQ ID NO:7 |
| CTPM2_ | TNEAKSLEAQ | AEKYSQKEDK | YEEEIKVLTD | KLKEAETRAE | FAERSVTKLE | 250 | SEQ ID NO:3 |
| hTPM3_ | TNNLKSLEAQ | AEKYSQKEDK | YEEEIKILTD | KLKEAETRAE | FAERSVAKLE | 250 | SEQ ID NO:9 |
| hTPM4_ | TNNLKSLEAA | SEKYSEKEDK | YEEEIKLLSD | KLKEAETRAE | FAERTVAKLE | 214 | SEQ ID NO:11 |
| hTPMB_ | TNNLKSLEAQ | ADKYSTKEDK | YEEEIKLLEE | KLKEAETRAE | FAERSVAKLE | 250 | SEQ ID NO:13 |
| hTPMF_ | DQTLKALMAA | EDKYSQKEDR | YEEEIKVLSD | KLKEAETRAE | FAERSVTKLE | 250 | SEQ ID NO:15 |
| hTPMN_ | DQNLKCLSAA | EEKYSQKEDK | YEEEIKILTD | KLKEAETRAE | FAERSVAKLE | 214 | SEQ ID NO:17 |
| hTPMS_ | DSDLESINAA | EDKYSQKEDR | YEEEIKVLSD | KLKEAETRAE | FAERSVTKLE | 193 | SEQ ID NO:19 |
| hTPM1_ | KSIDDLEDEL | YAQKLKYKAI | SEELDHALND | MTSI | | 284 | SEQ ID NO:5 |
| cTPM1_ | KTIDDLEESL | ASAKEENVGI | HQVLDQTLLE | LNNL | | 284 | SEQ ID NO:1 |
| hTPM2_ | KTIDDLEETL | ASAKEENVEI | HQTLDQTLLE | LNNL | | 284 | SEQ ID NO:7 |
| cTPM2_ | KSIDDLEEKV | AHAKEENLNM | HQMLDQTLLE | LNNM | | 284 | SEQ ID NO:3 |
| hTPM3_ | KTIDDLEDEL | YAQKLKYKAI | SEELDHALND | MTSI | | 284 | SEQ ID NO:9 |
| hTPM4_ | KTIDDLEEKL | AQAKEENVGL | HQTLDQTLNE | LNCI | | 248 | SEQ ID NO:11 |
| hTPMB_ | KTIDDLEDEV | YAQKMKYKAI | SEELDNALND | ITSL | | 284 | SEQ ID NO:13 |
| hTPMF_ | KSIDDLEEKV | AHAKEENLSM | HQMLDQTLLE | LNNM | | 284 | SEQ ID NO:15 |
| hTPMN_ | KTIDDLEDKL | KCTKEEHLCT | QRMLDQTLLD | LNEM | | 248 | SEQ ID NO:17 |
| hTPMS_ | KSIDDLEEKV | AHAKEENLSM | HQMLDQTLLE | LNNM | | 227 | SEQ ID NO:19 |

TABLE 4

| | | | | | |
|---|---|---|---|---|---|
| 10 | 20 | 30 | 40 | 50 | 60 |
| SESVKEAQEK | LEQAEKKATD | AEAEVASLNR | RIQLVEEELD | RAQERLATAL | QKLEEAEKAA |
| LEELHKSEDS | LLSAEENAAK | AESEVASLNR | RIQLVEEELD | RAQERLATAL | QKLEEAEKAA |
| SEALKDAQEK | LELAEKKATD | AEADVASLNR | RIQLVEEELD | RAQERLATAL | QKLEEAEKAA |
| SESVKEAQEK | LEQAEKKATD | AEADVASLNR | RIQLVEEELD | RAQERLATAL | QKLEEAEKAA |
| SEALKQAQEK | LELAEKKAAD | AEAEVASLNR | RIQLVEEELD | RAQERLATAL | QKLEEAEKAA |
| EDRAQGLQRE | LDGERERREK | AEGDVAALNR | RIQLVEEELD | RAQERLATAL | QKLEEAEKAA |
| SESVKEAQEK | LEQAEKKATD | AEADVASLNR | RIQLVEEELD | RAQERLATAL | QKLEEAEKAA |
| SEALKDAQEK | LELAEKKATD | AEADVASLNR | RIQLVEEELD | RAQERLATAL | QKLEEAEKAA |
| EERAERLQRE | VEGERRAREQ | AEAEVASLNR | RIQLVEEELD | RAQERLATAL | QKLEEAEKAA |
| RIFLRTASSE | HLHERKLRET | AEADVASLNR | RIQLVEEELD | RAQERLATVL | QKLEEAEKAA |
| 70 | 80 | 9 | 100 | 110 | 120 |
| DESERGMKVI | ENRAMKDEEK | MELQEMQLKE | AKHIAEEADR | KYEEVARKLV | VLEGELERSE |
| DESERGMKVI | ENRAQKDEEK | MEIQEIQLKE | AKHIAEEADR | KYEEVARKLV | ILEGDLERAE |
| DESERGMKVI | ESRAQKDEEK | MEIQEIQLKE | AKHIAEDADR | KYEEVARKLV | IIESDLERAE |
| DESERGMKVI | ENRAMKDEEK | MELQEMQLKE | AKHIAEDSDR | KYEEVARKLV | ILEGELERSE |
| DESERGMKVI | ENRALKDEEK | MELQEIQLKE | AKHIAEEADR | KYEEVARKLV | IIEGDLERTE |
| DESERGMKVI | ENRAMKDEEK | MEIQEMQLKE | AKHIAEEADR | KYEEVARKLV | ILEGELERAE |
| DESERGMKVI | ENRAMKDEEK | MELQEMQLKE | AKHIAEDSDR | KYEEVARKLV | ILEGELERSE |
| DESERGMKVI | ESRAQKDEEK | MEIQEIQLKE | AKHIAEDADR | KYEEVARKLV | IIESDLERAE |
| DESERGMKVI | ENRALKDEEK | MELQEIQLKE | AKHIAEEADR | KYEEVARKLV | IIEGDLERTE |
| DESERGMKVI | ESRAQKDEEK | MEIQEIQLKE | AKHIAEDADR | KYEEVARKLV | IIESDLERAE |
| 130 | 140 | 150 | 154 | | |
| ERAEVAESRV | RQLEEELRTM | DQSLKSLIAS | EEEY | | SEQ ID NO:2 |
| ERAELSESKC | AELEEELKLV | TNEAKSLEAQ | AEKY | | SEQ ID NO:4 |
| ERAELSEGKC | AELEEELKTV | TNNLKSLEAQ | AEKY | | SEQ ID NO:6 |
| ERAEVAESRA | RQLEEELRTM | DQALKSLMAS | EEEY | | SEQ ID NO:8 |
| ERAELAESKC | SELEEELKNV | TNNLKSLEAQ | AEKY | | SEQ ID NO:10 |
| ERAEVSELKC | GDLEEELKNV | TNNLKSLEAA | SEKY | | SEQ ID NO:12 |
| ERAEVAESKC | GDLEEELKIV | TNNLKSLEAQ | ADKY | | SEQ ID NO:14 |
| ERAELSEGQV | RQLEEQLRIM | DQTLKALMAA | EDKY | | SEQ ID NO:16 |
| ERAELAESRC | REMDEQIRLM | DQNLKCLSAA | EEKY | | SEQ ID NO:18 |
| ERAELSEGQV | RQLEEQLRIM | DSDLESINAA | EDKY | | SEQ ID NO:20 |

The present invention includes conservative amino acid substitution variants of any of the above polypeptides. Also included in the invention is a set of overlapping peptides having at least 4 amino acids, preferably between about 10 and about 40 amino acids, which are derived from SEQ ID NO:2, 4, 6, 8, 10, 12, 14, 16 18 or 20, or conservative substitution variants thereof. The polypeptides and peptides of the present invention are characterized in that they either (a) bind directly to an angiogenic polypeptide or peptide such as HKa D5 or HPRG or an angiogenic peptide fragment of HPRG or (b) inhibit the binding of such an angiogenic peptide to a Tpm protein which is either expressed on a cell surface, immobilized to a solid support, or in solution in a competitive binding assay such as is described herein.

In another embodiment, the invention encompasses peptides which are homologous to a human Tpm, preferably SEQ ID NO:5, 7 or 9 or fragments thereof, preferably SEQ ID NO:6, 8 or 10. In one embodiment, the amino acid sequence of the peptide is at least 70% identical to the sequence of a wild type fragment of human Tpm. Preferably the identity is at least 85%., more preferably, at least 90%.

In the case of the cTPM1 polypeptide SEQ ID NO:2, the TM-311 mAb does not recognize this fragment. It is noteworthy that the TM-311 mAb is not anti-angiogenic in human systems that have been tested such as HUVEC or a CAM assay, even though this mAb blocks HKa binding of Tpm. This mAb is antiangiogenic and has anti-tumor activity in murine systems.

The present invention is directed to antibodies, preferably mAbs that directly bind Tpm or a Tpm polypeptide or peptide such as AALBP as described above and act as inhibitors or antagonists of angiogenesis by evoking an antiangiogenic signal in a cell to which they bind via cell surface Tpm or bind to Tpm expressed on the surface of angiogenic ECs.

Although the inventors do not wishing to be bound by any particular mechanistic explanation for the results disclosed herein, they have conceived that the compositions and methods of the present invention exert antitumor effects either by (1) antiangiogenic effects mediated via generation of signal in EC's that lead to apoptosis, (2) direct apoptotic signals to tumor cells that express a Tpm polypeptide on their surface or (3) by both mechanisms.

According to this invention, any protein that can bind to cell surface Tpm and initiate an apoptotic pathway or otherwise inhibit angiogenesis can be used to treat conditions associated with undesired angiogenesis, for example, tumor growth and metastasis. The following is a nonlimiting list of Tpm-binding proteins (see, e.g., Perry S V, J Muscle Res. Cell Motil. (2001), 22:5-49; Squire J M et al., FASEB J. (1998) 12:761-771; Lin J J et al., Int. Rev. Cytol. (1997).170:1-38) that can be used in accordance with this invention to inhibit angiogenesis: troponin T (Smillie, L B, Trends Biochem Sci (1979). 4:151-155; Zot A S et al., Annu Rev Biophys Chem (1987) 16:535-559); tropomodulin (Fowler V M, J Biol Chem (1987) 262:12792-800); caldesmon (Marston S B et al., Biochem J (1991): 279:1-16); calponin (Gimona M et al., In: Barany (ed), Biochemistry of Smooth Muscle Contraction, pp 91-103, Academic Press, New York. (1996); Takahashi K et al., Biochem Biophys Res Commun (1986) 141: 20-26); pEL98 (Takenaga K et al., J Cell Biol (1994) 124:757-768); glutamic dehydrogenase (Akutsu S et al., Zool Sci (2000).17: 871-879) and actin.

Troponin T with two other molecules, Troponin I and C, form a trimer that binds to Tpm. Troponin T is the subunit responsible for Tpm binding. U.S. Pat. No. 6,025,331 (Moses, et al., Feb. 15, 2000) disclosed that all three of these proteins could inhibit bovine capillary EC proliferation induced by bFGF. This document indicated that these proteins could be used to inhibit angiogenesis. Moses, M A et al., 1999, Proc. Natl. Acad. Sci. USA 96:2645-2650, disclosed that human cartilage Troponin I, purified to apparent homogeneity, is a potent and specific inhibitor of angiogenesis in vivo and in vitro, as well as of tumor metastasis in vivo using the B16-BL6 murine melanoma model. The mechanism(s) by which Troponin I inhibited capillary EC proliferation in vitro and angiogenesis in vivo was said by these authors to be unknown although the authors suggested a connection with the known effects of modulation of EC cell shape via manipulation of the cytoskeleton and its regulatory elements, including actin-associated proteins, on EC cell growth and capillary morphogenesis. The existence of an EC Troponin I receptor was suggested as a means by which this protein could induce changes in EC shape that, in turn, would suppress growth of these cells. It was further suggested that, under physiological conditions, Troponin I (pI 8.88), with its relatively rich lysine content, could, through affinity for heparin, bind to and compete with bFGF and perhaps VEGF for heparin sulfate proteoglycan on the EC surface.

As noted above, Troponin I does not bind to Tpm. Rather, Troponin T is the subunit that binds tropomyosin whereas Troponin I had the antiangiogenic activity in the above study.

Tropomyosin-Binding Angiogenesis Inhibitors

According to the present invention, Tpm is targeted by several anti-angiogenic proteins. These are described below.

1. Histidine Proline Rich Glycoprotein and Fragments Thereof

These polypeptides and their antiangiogenic properties are described in detail in copending, commonly assigned U.S. patent application Ser. No. 10/074,225, filed 14 Feb. 2001, which is hereby incorporated by reference in its entirety.

Full Length Human HPRG has the Amino Acid Sequence SEQ ID NO:21

| | | | | | | |
|---|---|---|---|---|---|---|
| MKALIAALLL | ITLQYSCAVS | PTDCSAVEPE | AEKALDLINK | RRRDGYLFQL | LRIADAHLDR | 60 |
| VENTTVYYLV | LDVQESDCSV | LSRKYWNDCE | PPDSRRPSEI | VIGQCKVIAT | RHSHESQDLR | 120 |
| VIDFNCTTSS | VSSALANTKD | SPVLIDFFED | TERYRKQANK | ALEKYKEEND | DFASFRVDRI | 180 |
| ERVARVRGGE | GTGYFVDFSV | RNCPRHHFPR | HPNVFGFCRA | DLFYDVEALD | LESPKNLVIN | 240 |
| CEVFDPQEHE | NINGVPPHLG | HPFHWGGHER | SSTTKPPFKP | HGSRDHHHPH | KPHEHGPPPP | 300 |
| PDERDHSHGP | PLPQGPPPLL | PMSCSSCQHA | TFGTNGAQRH | SHNNNSSDLH | PHKHHSHEQH | 360 |
| PHGHHPHAHH | PHEHDTHRQH | PHGHHPHGHH | PHGHHPHGHH | PHGHHPHCHD | FQDYGPCDPP | 420 |
| PHNQGHCCHG | HGPPPGHLRR | RGPGKGPRPF | HCRQIGSVYR | LPPLRKGEVL | PLPEANFPSF | 480 |
| PLPHHKHPLK | PDNQPFPQSV | SESCPGKFKS | GFPQVSMFFT | HTFPK | | 525 |

Rabbit HPRG has the Amino Acid Sequence SEQ ID NO:22 as Follows:

| | | | | | | |
|---|---|---|---|---|---|---|
| ATLQCSWALT | PTDCKTTKPL | AEKALDLINK | WRRDGYLFQL | LRVADAHLDG | AESATVYYLV | 60 |
| LDVKETDCSV | LSRKHWEDCD | PDLTKRPSLD | VIGQCKVIAT | RYSDEYQTLR | LNDFNCTTSS | 120 |
| VSSALANTKD | SPVLFDFIED | TEPFRKSADK | ALEVYKSESE | AYASFRVDRV | ERVTRVKGGE | 180 |
| RTNYYVDFSV | RNCSRSHFHR | HPAFGFCRAD | LSFDVEASNL | ENPEDVIISC | EVFNFEEHGN | 240 |
| ISGFRPHLGK | TPLGTDGSRD | HHHPHKPHKF | GCPPPQEGED | FSEGPPLQGG | TPPLSPPFRP | 300 |
| RCRHRPFGTN | ETHRFPHHRI | SVNIIHRPPP | HGHHPHGPPP | HGHHPHGPPP | HGHPPHGPPP | 360 |
| RHPPHGPPPH | GHPPHGPPPH | GHPPHGPPPH | GHPPHGPPPH | GHPPHGHGFH | DHGPCDPPSH | 420 |
| KEGPQDLHQH | AMGPPPKHPG | KRGPGKGHFP | FHWRRIGSVY | QLPPLQKGEV | LPLPEANFQP | 480 |
| LLLRNHTHPL | KPEIQPFPQV | ASERCPEEFN | GEFAQLSKFF | PSTFPK | | 526 |

italic: signal sequence
double underscore: Pro-rich domain

single underscore: His-Pro (H/P) rich domain

| The H/P domain of human HPRG has the sequence SEQ ID NO:23: | | | | | |
|---|---|---|---|---|---|
| HPHKHHSHEQ | HPHGHHPHAH | HPHEHDTHRQ | HPHGHHPHGH | HPHGHHPHGH | HPHGHHPHCH |
| DFQDYGPCDP | PPHNQGHCCH | GHGPPPGHLR | RRGPGKGPRP | FHCRQIGSVY | RLPPLRKGEV |
| LPLPEANFPS | FPLPHHKHPL | KPDNQPFP | | | |
| The H/P domain of rabbit HPRG has the sequence SEQ ID NO:24: | | | | | |
| SVNIIHRPPP | HGHHPHGPPP | HGHHPHGPPP | HGHPPHGPPP | RHPPHGPPPH | GHPPHGPPPH |
| GHPPHGPPPH | GHPPHGPPPH | GHPPHGHGFH | DHGPCDPPSHK | | |

2. Human Kininogen (HK) and Fragments

The full sequence of the mature form of HK (SEQ ID NO:25) is presented below.

| | | | | | | |
|---|---|---|---|---|---|---|
| QESQSEEIDC | NDKDLFKAVD | AALKKYNSQN | QSNNQFVLYR | ITEATKTVGS | DTFYSFKYEI | 60 |
| KEGDCPVQSG | KTWQDCEYKD | AAKAATGECT | ATVGKRSSTK | FSVATQTCQI | TPAEGPVVTA | 120 |
| QYDCLGCVHP | ISTQSPDLEP | ILRHGIQYFN | NNTQHSSLFM | LNEVKRAQRQ | VVAGLNFRIT | 180 |
| YSIVQTNCSK | ENFLFLTPDC | KSLWNGDTGE | CTDNAYIDIQ | LRIASFSQNC | DIYPGKDFVQ | 240 |
| PPTKICVGCP | RDIPTNSPEL | EETLTHTITK | LNAENNATFY | FKIDNVKKAR | VQVVAGKKYF | 300 |
| IDFVARETTC | SKESNEELTE | SCETKKLGQS | LDCNAEVYVV | PWEKKIYPTV | NCQPLGMISL | 360 |
| MKRPPGFSPF | RSSRIGEIKE | ETTVSPPHTS | MAPAQDEERD | SGKEQGHTRR | HDWGHEKQRK | 420 |
| HNLGHGHKHE | RDQGHGHQRG | HGLGHGHEQQ | HGLGHGHKFK | LDDDLEHQGG | HVLDHGHKHK | 480 |
| HGHGHGKHKN | KGKKNGKHNG | WKTEHLASSS | EDSTTPSAQT | QEKTEGPTPI | PSLAKPGVTV | 540 |
| TFSDFQDSDL | IATMMPPISP | APIQSDDDWI | PDIQTDPNGL | SFNPISDFPD | TTSPKCPGRP | 600 |
| WKSVSEINPT | TQMKESYYFD | LTDGLS | | | | 626 |

The D5 domain of HK (amino acid residues 384-508 of the mature HK sequence; underscored above) is useful as an inhibitor of angiogenesis and of various EC functions including cell proliferation as disclosed in commonly assigned application of some of the present inventors, PCT/US01/23185, filed Jul. 24, 2001, “Human Kininogen D5 Domain Polypeptides” (A. Mazar & Jose Juarez), incorporated by reference in its entirety.

The 125 residue D5 domain has the sequence SEQ ID NO:26:

| | | | | | |
|---|---|---|---|---|---|
| VSPPHTSMAP | AQDEERDSGK | EQGHTRRHDW | GHEKQRKHNL | GHGHKHERDQ | 50 |
| GHGHQRGHGL | GHGHEQQHGL | GHGHKFKLDD | DLEHQGGHVL | DHGHKHKHGH | 100 |
| GHGKHKNKGK | KNGKHNGWKT | EHLAS | | | 125 |

The present invention is also directed to functional homologues of human or chicken Tpm or the AALBP fragment thereof. A functional homologue must possess the biochemical and biological activity, preferably anti-angiogenic and anti-tumor activity which can be tested using in vitro or in vivo methods described herein. In view of this functional characterization, use of homologous Tpm proteins from other species, including proteins not yet discovered, falls within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. For example, preferred alignment would be with human Tpm hTPM2 (SEQ ID NO:7) or its fragment SEQ ID NO:8, at least 30%, preferably at least 40%, more preferably at least 50%, even more preferably at least 60% and even more preferably at least 70, 80 or 90% of the amino acid residues are aligned. The amino acid residues (or nucleotides from the coding sequence) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acids encoding the present polypeptide sequences and the polypeptide sequences of this invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to human or murine HPRG nucleic acid molecules. BLAST protein searches can be performed with the XBLAST program, score=50, wordlength=3 to obtain amino acid sequences homologous to HPRG protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.k.

Thus, a homologue of a particular isoform of a human Tpm described above is characterized as having (a) functional activity of native Tpm or a ligand-binding fragment thereof, and (b) sequence similarity to a native Tpm when determined above, of at least about 30% (at the amino acid level), preferably at least about 50%, more preferably at least about 70%, even more preferably at least about 90%.

It is within the skill in the art to obtain and express such a protein using DNA probes based on the disclosed sequences of the Tpm's. Then, the protein's biochemical and biological activity can be tested readily using art-recognized methods such as those described herein.

Peptide Compositions

A preferred composition is, or comprises, a biologically active peptide of Tpm characterized in that it possesses the binding and/or biological activity of Tpm. Such binding is to a ligand that is preferably an antiangiogenic protein or peptide that interacts with EC's and promotes their apoptosis or otherwise generates a signal that downregulates any EC function associated with angiogenesis.

Moreover, a biologically active peptide has Tpm-like activity in an in vitro or in vivo assay of binding or of biological activity such as those characterized herein. Preferably the peptide blocks binding of HPRG or HKa D5 or anti-Tpm mAbs to (a) EC's via cell surface Tpm or (b) isolated Tpm in a direct binding assay. A preferred peptide comprises a minimal sequence needed to bind to an antiangiogenic anti-Tpm mAb.

The peptide may be capped at its N and C termini with an acyl (abbreviated “Ac”)—and an amido (abbreviated “Am”) group, respectively, for example acetyl (CH₃CO—) at the N terminus and amido (—NH₂) at the C terminus.

A broad range of N-terminal capping functions, preferably in a linkage to the terminal amino group, is contemplated, for example:
formyl;
alkanoyl, having from 1 to 10 carbon atoms, such as acetyl, propionyl, butyryl;
alkenoyl, having from 1 to 10 carbon atoms, such as hex-3-enoyl;
alkynoyl, having from 1 to 10 carbon atoms, such as hex-5-ynoyl;
aroyl, such as benzoyl or 1-naphthoyl;
heteroaroyl, such as 3-pyrroyl or 4-quinoloyl;
alkylsulfonyl, such as methanesulfonyl;
arylsulfonyl, such as benzenesulfonyl or sulfanilyl;
heteroarylsulfonyl, such as pyridine-4-sulfonyl;
substituted alkanoyl, having from 1 to 10 carbon atoms, such as 4-aminobutyryl;
substituted alkenoyl, having from 1 to 10 carbon atoms, such as 6-hydroxy-hex-3-enoyl;
substituted alkynoyl, having from 1 to 10 carbon atoms, such as 3-hydroxy-hex-5-ynoyl;
substituted aroyl, such as 4-chlorobenzoyl or 8-hydroxy-naphth-2-oyl;
substituted heteroaroyl, such as 2,4-dioxo-1,2,3,4-tetrahydro-3-methyl-quinazolin-6-oyl;
substituted alkylsulfonyl, such as 2-aminoethanesulfonyl;
substituted arylsulfonyl, such as 5-dimethylamino-1-naphthalenesulfonyl;
substituted heteroarylsulfonyl, such as 1-methoxy-6-isoquinolinesulfonyl; carbamoyl or thiocarbamoyl;
substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl;
substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

The C-terminal capping function can either be in an amide or ester bond with the terminal carboxyl. Capping functions that provide for an amide bond are designated as NR¹R² wherein R¹ and R² may be independently drawn from the following group:
hydrogen;
alkyl, preferably having from 1 to 10 carbon atoms, such as methyl, ethyl, isopropyl;
alkenyl, preferably having from 1 to 10 carbon atoms, such as prop-2-enyl;
alkynyl, preferably having from 1 to 10 carbon atoms, such as prop-2-ynyl;
substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;
substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;
substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;
aroylalkyl having up to 10 carbon atoms, such as phenacyl or 2-benzoylethyl;
aryl, such as phenyl or 1-naphthyl;
heteroaryl, such as 4-quinolyl;
alkanoyl having from 1 to 10 carbon atoms, such as acetyl or butyryl;
aroyl, such as benzoyl;
heteroaroyl, such as 3-quinoloyl;
OR′ or NR′R″ where R′ and R″ are independently hydrogen, alkyl, aryl, heteroaryl, acyl, aroyl, sulfonyl, sulfinyl, or SO₂—R′″ or SO—R″ where R′″ is substituted or unsubstituted alkyl, aryl, heteroaryl, alkenyl, or alkynyl.

Capping functions that provide for an ester bond are designated as OR, wherein R may be: alkoxy; aryloxy, heteroaryloxy; aralkyloxy; heteroaralkyloxy; substituted alkoxy; substituted aryloxy; substituted heteroaryloxy; substituted aralkyloxy; or substituted heteroaralkyloxy.

Either the N-terminal or the C-terminal capping function, or both, may be of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules.

Cyclic Peptides

Another class of peptides useful as angiogenesis inhibitors in accordance with the present invention are cyclic peptides (which can be considered peptide derivatives if they include additional chemical linkers). The cyclic peptides of this invention include a peptide that has between 4 and about 20 amino acids and has the property of binding to immobilized D5 domain of HKₐ (see above) or larger forms of a that include D5. The cyclic peptide must also inhibit angiogenesis in any of the known direct or surrogate assays described herein.

Methods for production of cyclic peptides, cyclization of linear peptides, etc., are well-known in the art and are therefore not set forth in detail herein. See, for example, Jones et al., U.S. Pat. No. 5,942,492; Mazar et al., U.S. Pat. No. 6,277,818; U.S. application Ser. No. 09/704,731, filed Nov. 3, 2000, (and which claims priority from the applications giving rise to the above two patents or from continuations thereof)). The contents of these patents/application are incorporated by reference in their entirety.

A general formula of an 11-mer cyclic peptide is shown below: 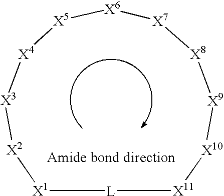
In this general formula, the amide bond (CO—NH) linking X¹ to X², is such that the carbonyl moiety is from amino acid X¹ and the amino moiety is from the amino acid X². The same is true for the link between X² and X³, and so on within this 11 mer peptide. The peptide has X¹ as its N-terminus and X¹¹ as its C-terminus. In a preferred embodiment, X¹ and Xⁿ (the C-terminal residue in the linear formula of the peptide) are Cys residues joined by a disulfide bond, which cyclizes the peptide. In such a case, no additional linker (L) is necessary. Other embodiments employ linkers which are discussed below.

To prepare a compound of the above formula, L is chosen to provide, at one terminus, a functional group that can be chemically bonded to the carboxyl C atom of amino acid X¹¹ and, at the other terminus, a functional group that can be chemically bonded to the α-amino N atom of amino acid X¹.

It is preferred that the linker L confer water solubility to the peptide and result in an intramolecular distance of 4-12 Å between the Cα of the N-terminal residue X¹ and the Cα of the C-terminal residue X¹¹.

Alternatively (again using an 11mer only as an example) the linear peptide X¹—X²—X³—X⁴—X⁵—X⁶—X⁷—X⁸—X⁹—X¹⁰—X¹¹ can be synthesized with an extension at X¹¹ comprising a portion of the ultimate final linker group L; that extension is termed L_{b}. After synthesis of the peptide chain, the X₁ terminus is extended with an extension that will also become part of the ultimate linker; this group is designated Lₐ. These steps yield a compound of the formula:
Lₐ—X¹—X²—X³—X⁴—X⁵—X⁶—X⁷—X⁸—X⁹—X¹⁰—X¹¹—L_{b}.
The free ends of Lₐ and L_{b} are then chemically bonded to each other. In this way, the linker L is formed during the cyclization step from pre-attached fragments Lₐ and L_{b}. In the examples given below for L, the direction of L, reading left to right, is from to X¹ to X¹¹, i.e., the C-terminus of L is bonded to X¹, and the N-terminus of L is bonded to X¹¹.

When L includes a Cys, HomoCys, Glu, Asp, γ-carboxyl modified Glu or a β-carboxyl modified Asp residue, the configuration of the enantiomeric center of such a residue can be either L- or D-.

Examples of Useful Linkers Follow.
L1—CO—CH₂—NH—CO—CH₂—CH₂—CH(CO—NH—CH₂—CO—NH₂)—NH—
L2-CO—CH₂—NH—CO—CH₂—CH₂—CH(CO—NH—CH(CH₂SH)—CO—NH₂)—NH—
L3-CO—CH(CH₂SH)—NH—CO—CH₂—CH₂—CH(CO—NH—CH₂—CONH₂)—NH—
L4-CO—CH₂—NH—CO—CH₂—CH₂—CH(CO—NH—CH(CH₂CH₂SH)—CO—NH₂)—NH—
L5-CO—CH(CH₂CH₂SH)—NH—CO—CH₂—CH₂—CH(CO—NH—CH₂—CONH₂)—NH—
L6 —CO—CH(CH₂CH₂COR¹)—NH—CO—CH₂—CH₂—CH(CO—NH—CH₂—CONH₂)—NH—
L7 —CO—CH(CH₂COR¹)—NH—CO—CH₂—CH₂—CH(CO—NH—CH₂—CONH₂)—NH—
L8 —CO—CH₂—NH—CO—CH₂—CH₂—CH(CO—NH—CH(CH₂CH₂COR¹)—CO—NH₂)—NH—
L9 —CO—CH₂—NH—CO—CH₂—CH₂—CH(CO—NH—CH(CH₂COR¹)—CO—NH₂)—NH—
L10 —CO—CH₂—NH—CO—CH₂—CH₂—CH(CO—NH—CH₂—COR¹)—NH—
L11 —CO—CH(CH₂CH₂COOH)—NH—CO—CH₂—CH₂—CH(CO—NH—CH₂—CONH₂)—NH—
L12 —CO—CH(CH₂COOH)—NH—CO—CH₂—CH₂—CH(CO—NH—CH₂—CONH₂)—NH—
L13 —CO—CH₂—NH—CO—CH₂—CH₂—CH(CO—NH—CH(CH₂CH₂COOH)—CO—NH₂)—NH—
L14 —CO—CH₂—NH—CO—CH₂—CH₂—CH(CO—NH—CH(CH₂COOH)—CO—NH₂)—NH—
L15 —CO—CH₂—NH—CO—CH₂—CH₂—CH(CO—NH—CH₂—CO—NH—R¹)—NH—

The R¹ group in L6-L10 is may be a weakly basic diamino group —NH—R²—NH₂, where the pKₐ of each of the primary amino groups in the parent diamine H₂N—R²—NH₂ is less than about 8.0 and where the pKₐ of the primary amino group in —NH—R²—NH₂, when it is part R¹ is also less than about 8.0. Preferred examples of R² are p-phenylene, o-phenylene or m-phenylene.

For introducing the R¹ group into linker L15 that is part of cyclic peptide of this invention, a weakly basic amine R¹NH₂ is preferably bonded to the glycine “spur” (which is the underscored part of L15 shown above). Amines intended for this linker are amine are not specifically limited by structure. Rather, the only requirement is that the pKₐ of its amino group be less than about 8.0. Aniline is a simple and prototypic example of a weakly basic amine, in fact, of the class of aromatic amines that are, in general, always weakly basic. To introduce an aromatic R¹ group, an aromatic amine is used. R¹ may be a homoaryl or a heteroaryl residue, and may be substituted with one or more substituents drawn from a broad range. The aromatic group may be polycyclic, wherein the various rings may be fused, unfused, or even both fused and unfused. In a polycyclic aromatic group, the rings may be homocyclic or heterocyclic, or even a mixture of both. The ring may be substituted with one or more substituents drawn from a broad range. In a preferred embodiment R¹ in L15 is phenyl or substituted phenyl.

The R¹ group of L15 need not be an aromatic residue to have the requisite property of weak basicity. For example, the class of amines comprise any aromatic residue substituted with an ω-(aminooxy)ₙ-alkyl group of 1 to 10 carbons—this is described by the formula: H₂N—O—(CH₂)ₓ— (where x=1-10). Another class of suitable amines are those having the formula H₂N—CH₂—CO—NH—(CH₂)ₓ-homoaryl or H₂N—CH₂—CO—NH—(CH₂)ₓ-heteroaryl, wherein x=2-10. The homoaryl or heteroaryl residue may be substituted with one or more substituents drawn from a broad range. As above, the homoaryl residue may be polycyclic, fused or unfused or both. The heteroaryl reside may additionally contain a homocylic ring or more than one homocyclic rings that may be fused, unfused or even both fused and unfused. These compounds described above are non-limiting and are illustrative of the broad structural nature that can be the property of a weakly basic amine included within th scope of this invention.

Three preferred cyclic peptides and results of studies using them are described in Example VII.

Production of Peptides and Derivatives

General Chemical Synthetic Procedures

The peptides of the invention may be prepared using recombinant DNA technology. However, given their length, they are preferably prepared using solid-phase synthesis, such as that generally described by Merrifield, J. Amer. Chem. Soc., 85:2149-54 (1963), although other equivalent chemical syntheses known in the art are also useful. Solid-phase peptide synthesis may be initiated from the C-terminus of the peptide by coupling a protected α-amino acid to a suitable resin. Such a starting material can be prepared by attaching an α-amino-protected amino acid by an ester linkage to a chloromethylated resin or to a hydroxymethyl resin, or by an amide bond to a BHA resin or MBHA resin.

Such methods, well-known in the art, are disclosed, for example, in U.S. Pat. No. 5,994,309 (issued Nov. 30, 1999) which is incorporated by reference in its entirety.

Amino Acid Substitution and Addition Variants

Also included in this invention are peptides in which at least one amino acid residue and preferably, only one, has been removed and a different residue inserted in its place compared to the native sequence. For a detailed description of protein chemistry and structure, see Schulz, G E, et al., Principles of Protein Structure, Springer-Verlag, New York, 1979, and Creighton, T. E., Proteins: Structure and Molecular Principles, W.H. Freeman & Co., San Francisco, 1984, which are hereby incorporated by reference. The types of substitutions which may be made in the peptide molecule of the present invention are conservative substitutions and are defined herein as exchanges within one of the following groups:
1. Small aliphatic, nonpolar or slightly polar residues: e.g., Ala, Ser, Thr, Gly;
2. Polar, negatively charged residues and their amides: e.g., Asp, Asn, Glu, Gln;
3. Polar, positively charged residues: e.g., His, Arg, Lys;
   Pro, because of its unusual geometry, tightly constrains the chain. Substantial changes in functional properties are made by selecting substitutions that are less conservative, such as between, rather than within, the above groups (or two other amino acid groups not shown above), which will differ more significantly in their effect on maintaining (a) the structure of the peptide backbone in the area of the substitution (b) the charge or hydrophobicity of the molecule at the target site, or (c) the bulk of the side chain. Most substitutions according to the present invention are those that do not produce radical changes in the characteristics of the peptide molecule. Even when it is difficult to predict the exact effect of a substitution in advance of doing so, one skilled in the art will appreciate that the effect can be evaluated by routine screening assays, preferably the biological assays described below. Modifications of peptide properties including redox or thermal stability, hydrophobicity, susceptibility to proteolytic degradation or the tendency to aggregate with carriers or into multimers are assayed by methods well known to the ordinarily skilled artisan.

The present invention provides methods to inhibit or reduce angiogenesis, tumor growth, EC proliferation, EC migration or EC tube formation or to induce EC apoptosis.

The invention also provides pharmaceutical compositions comprising fragments, peptides, conformers, antibodies against, biological equivalents of or derivatives of Tpm or AALBP.

The AALBP may be obtained from Tpm isolated from any appropriate tissue source such as tissue extracts or as a product of a cell line growing in culture that produces “native” Tpm, preferably cell surface Tpm, or the AALBP fragment of the Tpm, or a “nonnative” Tpm or AALBP that has been genetically modified, or a functional derivative thereof, such that such cells express this polypeptide or a functional derivative thereof such as a domain or shorter fragment.

Tpm fragments or derivatives are chemically synthesized, or produced by recombinant methods. Recombinant techniques known in the art include, but are not limited to DNA amplification using PCR of a cDNA library for example by reverse transcription of mRNA in cells extracts followed by PCR.

Basic texts disclosing general methods of molecular biology, all of which are incorporated by reference, include: Sambrook, J. et al., Molecular Cloning: A Laboratory Manual, 2^{nd} Edition, Cold Spring Harbor Press, Cold Spring Harbor, N.Y., 1989; Ausubel, F. M. et al. Current Protocols in Molecular Biology, Vol. 2, Wiley-Interscience; New York, (current edition); Kriegler, Gene Transfer and Expression: A Laboratory Manual (1990); Glover, D. M., ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J. D. et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Fragments of Tpm are be obtained by controlled protease reaction (Borza D-B. et al., Biochemistry, 1996, 35; 1925-1934). Chymotrypsin digestion is exemplified herein. Alternatively, Tpm can be subjected to limited plasmin digestion followed by partial reduction with dithiothreitol to create fragments of Tpm bind and block the action of Tpm-binding antiangiogenic agents. These may be useful in situations where it is desirable to promote angiogenesis by blocking endogenous homeostatic mechanisms that might otherwise limit it.

Chemical Derivatives of Tpm and AALBP

“Chemical derivatives” of Tpm or AALBP contain additional chemical moieties not normally a part of the protein. Covalent modifications of the polypeptide are included within the scope of this invention. Such derivatized moieties may improve the solubility, absorption, biological half life, and the like. Moieties capable of mediating such effects are disclosed, for example, in Remington's Pharmaceutical Sciences, 16^{th} ed., Mack Publishing Co., Easton, Pa. (1980).

Such modifications may be introduced into the molecule by reacting targeted amino acid residues of the polypeptide with an organic derivatizing agent that is capable of reacting with selected side chains or terminal residues. Another modification is cyclization of the protein.

Cysteinyl residues most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenyl, or chloro-7-nitrobenzo-2-oxa-1,3-diazole.

Histidyl residues are derivatized by reaction with diethylprocarbonate (pH 5.5-7.0) which agent is relatively specific for the histidyl side chain p-bromophenacyl bromide also is useful; the reaction is preferably performed in 0.1 M sodium cacodylate at pH 6.0.

Lysinyl and amino terminal residues are derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKₐ of the guanidine functional group. Furthermore, these reagents may react with the groups of lysine as well as the arginine ε-amino group.

Modification of tyrosyl residues has permits introduction of spectral labels into a peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively.

Carboxyl side groups, aspartyl or glutamyl, may be selectively modified by reaction with carbodiimides (R—N═C═N—R′) such as 1-cyclohexyl-3-(2-morpholinyl-(4-ethyl)carbodiimide or 1-ethyl-3-(4-azonia-4,4-dimethylpentyl)carbodiimide. Furthermore, aspartyl and glutamyl residues can be converted to asparaginyl and glutaminyl residues by reaction with ammonia.

Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention.

Derivatization with bifunctional agents is useful for cross-linking the peptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of the hydroxyl groups of seryl or threonyl residues, methylation of the α-amino groups of lysine, arginine, and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl groups.

Also included are peptides wherein one or more D-amino acids are substituted for one or more L-amino acids.

Diagnostic and Prognostic Compositions

The peptides of the invention can be detectably labeled and used, for example, to detect a peptide binding protein ligand or a cellular binding site/receptor (such as the binding sites on activated ECs, tumor cells, etc.) whether on the surface or in the interior of a cell. The fate of the peptide during and after binding can be followed in vitro or in vivo by using the appropriate method to detect the label. The labeled peptide may be utilized in vivo for diagnosis and prognosis, for example to image occult metastatic foci or for other types of in situ evaluations.

The term “diagnostically labeled” means that the polypeptide or peptide has attached to it a diagnostically detectable label. There are many different labels and methods of labeling known to those of ordinary skill in the art, described below. General classes of labels which can be used in the present invention include radioactive isotopes, paramagnetic isotopes, and compounds which can be imaged by positron emission tomography (PET), fluorescent or colored compounds, etc. Suitable detectable labels include radioactive, fluorescent, fluorogenic, chromogenic, or other chemical labels. Useful radiolabels (radionuclides), which are detected simply by gamma counter, scintillation counter or autoradiography include ³H, 125I, ¹³¹I, ³⁵S and ¹⁴. ¹³¹I is also a useful therapeutic isotope (see below).

A number of U.S. patents, incorporated by reference herein, disclose methods and compositions for complexing metals to larger molecules, including description of useful chelating agents. The metals are preferably detectable metal atoms, including radionuclides, and are complexed to proteins and other molecules. These documents include: U.S. Pat. No. 5,627,286 (Heteroatom-bearing ligands and metal complexes thereof); U.S. Pat. No. 5,618,513 (Method for preparing radiolabeled peptides); U.S. Pat. No. 5,567,408; U.S. Pat. No. 5,443,816 (Peptide-metal ion pharmaceutical preparation and method); U.S. Pat. No. 5,561,220 (Tc-⁹⁹m labeled peptides for imaging inflammation).

Common fluorescent labels include fluorescein, rhodamine, dansyl, phycoerythrin, phycocyanin, allophycocyanin, o-phthaldehyde and fluorescamine. The fluorophore, such as the dansyl group, must be excited by light of a particular wavelength to fluoresce. See, for example, Haugland, Handbook of Fluorescent Probes and Research Chemicals, Sixth Ed., Molecular Probes, Eugene, Oreg., 1996). Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Similarly, fluorophores may also be coupled to thiols using maleimide, iodoacetamide, and aziridine-reactive groups. The long wavelength rhodamines, which are basically Rhodamine Green™ derivatives with substituents on the nitrogens, are among the most photostable fluorescent labeling reagents known. Their spectra are not affected by changes in pH between 4 and 10, an important advantage over the fluoresceins for many biological applications. This group includes the tetramethylrhodamines, X-rhodamines and Texas Red™ derivatives. Other preferred fluorophores for derivatizing the peptide according to this invention are those which are excited by ultraviolet light. Examples include cascade blue, coumarin derivatives, naphthalenes (of which dansyl chloride is a member), pyrenes and pyridyloxazole derivatives. Also included as labels are two related inorganic materials that have recently been described: semiconductor nanocrystals, comprising, for example, cadmium sulfate (Bruchez, M. et al., Science 281:2013-2016 (1998), and quantum dots, e.g., zinc-sulfide-capped Cd selenide (Chan, W. C. W. et al., Science 281:2016-2018 (1998)).

In yet another approach, the amino group of the peptide is allowed to react with reagents that yield fluorescent products, for example, fluorescamine, dialdehydes such as o-phthaldial-dehyde, naphthalene-2,3-dicarboxylate and anthracene-2,3-dicarboxylate. 7-nitrobenz-2-oxa-1,3-diazole (NBD) derivatives, both chloride and fluoride, are useful to modify amines to yield fluorescent products.

The peptides of the invention can also be labeled for detection using fluorescence-emitting metals such as ¹⁵²Eu, or others of the lanthanide series. These metals can be attached to the peptide using such metal chelating groups as diethylenetriaminepentaacetic acid (DTPA, see Example X, infra) or ethylenediaminetetraacetic acid (EDTA). DTPA, for example, is available as the anhydride, which can readily modify the NH₂-containing peptides of this invention.

For in vivo diagnosis or therapy, radionuclides may be bound to the peptide either directly or indirectly using a chelating agent such as DTPA and EDTA. Examples of such radionuclides are ⁹⁹Tc, ¹²³I, ¹²⁵I, ¹³¹I, ¹¹¹In, ⁹⁷Ru, ⁶⁷Cu, ⁶⁷Ga, ⁶⁸Ga, ⁷²As, ⁸⁹Zr, ⁹⁰Y and ²⁰¹Tl. Generally, the amount of labeled peptide needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg.

The peptide can also be made detectable by coupling to a phosphorescent or a chemiluminescent compound. The presence of the chemiluminescent-tagged peptide is then determined by detecting the presence of luminescence that arises during the course of a chemical reaction. Examples of particularly useful chemiluminescers are luminol, isoluminol, theromatic acridinium ester, imidazole, acridinium salt and oxalate ester. Likewise, a bioluminescent compound may be used to label the peptides. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In yet another embodiment, colorimetric detection is used, based on chromogenic compounds which have, or result in, chromophores with high extinction coefficients.

In situ detection of the labeled peptide may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

For diagnostic in vivo radioimaging, the type of detection instrument available is a major factor in selecting a radionuclide. The radionuclide chosen must have a type of decay which is detectable by a particular instrument. In general, any conventional method for visualizing diagnostic imaging can be utilized in accordance with this invention. Another factor in selecting a radionuclide for in vivo diagnosis is that its half-life be long enough so that the label is still detectable at the time of maximum uptake by the target tissue, but short enough so that deleterious irradiation of the host is minimized. In one preferred embodiment, a radionuclide used for in vivo imaging does not emit particles, but produces a large number of photons in a 140-200 keV range, which may be readily detected by conventional gamma cameras.

In vivo imaging may be used to detect occult metastases which are not observable by other methods if such metastases express cell surface Tpm. Imaging could be used to stage tumors non-invasively or to detect other diseases which are associated with the presence of increased levels of surface Tpm by binding with anti-Tpm antibodies or ligands for the Tpm such as HPRG or HKa D5.

Peptidomimetics

A preferred type of chemical derivative of the peptides described herein is a peptidomimetic compound which mimics the biological effects of Tpm, AALBP or of a biologically active peptide thereof. A peptidomimetic agent may be an unnatural peptide or a non-peptide agent that recreates the stereospatial properties of the binding elements of Tpm such that it has the binding activity or biological activity of Tpm. Similar to biologically active peptides, a peptidomimetic will have a binding face (which interacts with any ligand to which Tpm binds) and a non-binding face. Again, similar to Tpm or its peptide, the non-binding face of a peptidomimetic will contain functional groups which can be modified by various therapeutic and diagnostic moieties without modifying the binding face of the peptidomimetic. A preferred embodiment of a peptidomimetic would contain an aniline on the non-binding face of the molecule. The NH₂-group of an aniline has a pKa˜4.5 and could therefore be modified by any NH₂— selective reagent without modifying any NH₂ functional groups on the binding face of the peptidomimetic. Other peptidomimetics may not have any NH₂ functional groups on their binding face and therefore, any NH₂, without regard for pKₐ, could be displayed on the non-binding face as a site for conjugation. In addition other modifiable functional groups, such as —SH and —COOH could be incorporated into the non-binding face of a peptidomimetic as a site of conjugation. A therapeutic or diagnostic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptide nature.

Peptidomimetic compounds, either agonists, substrates or inhibitors, have been described for a number of bioactive peptides such as opioid peptides, VIP, thrombin, HIV protease, etc. Methods for designing and preparing peptidomimetic compounds are known in the art (Hruby, V. J., Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that possess at least the binding capacity and specificity of the HPRG peptides and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the cystallographically-derived three-dimensional structure of a peptide of the invention either free or bound in complex with a ligand such as (a) heparin, plasminogen, fibrinogen, vitronectin and thrombospondin or (b) small ligands, such as heme and transition metal ions (zinc, copper and nickel). Alternatively, the structure of a peptide of the invention bound to its ligand can be gained by the techniques of nuclear magnetic resonance spectroscopy. The better knowledge of the stereochemistry of the interaction of the peptide with its ligand or receptor will permit the rational design of such peptidomimetic agents. The structure of a peptide or protein of the invention in the absence of ligand could also provide a scaffold for the design of mimetic molecules.

Antibodies Specific for Epitopes of Tropomyosin

The present invention provides antibodies, both polyclonal and monoclonal, reactive with an epitope of Tpm, preferably, an epitope of the AALBP fragment. These anti-Tpm antibodies may be xenogeneic, allogeneic, syngeneic, or modified forms thereof, such as humanized or chimeric antibodies. Antiidiotypic antibodies specific for the idiotype of an anti-Tpm antibody are also included.

In the following description, reference will be made to various methodologies known to those of skill in the art of immunology. Publications and other materials setting forth such known methodologies to which reference is made are incorporated herein by reference in their entireties as though set forth in full. Standard reference works setting forth the general principles of immunology include A. K. Abbas et al., Cellular and Molecular Immunology (Fourth Ed.), W. B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C. V. Mosby Co., St. Louis, Mo. (1999); Klein, J., Immunology, Blackwell Scientific Publications, Inc., Cambridge, Mass., (1990).

Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbiol., Immunol. Volume 119, 1985; Bona, C. et al., CRC Crit. Rev. Immunol., pp. 33-81 (1981); Jerne, N K, Ann. Immunol. 125C:373-389 (1974); Jerne, N K, In: Idiotypes-Antigens on the Inside, Westen-Schnurr, I., ed., Editiones Roche, Basel, 1982, Urbain, J et al., Ann. Immunol. 133D:179-(1982); Rajewsky, K. et al., Ann. Rev. Immunol. 1:569-607 (1983).

The term “antibody” is also meant to include both intact molecules as well as fragments thereof that include the antigen-binding site and are capable of binding to a Tpm epitope. These include, Fab and F(ab′)₂ fragments which lack the Fc fragment of an intact antibody, clear more rapidly from the circulation, and may have less non-specific tissue binding than an intact antibody (Wahl et al., J. Nucl. Med. 24:316-325 (1983)). Also included are Fv fragments (Hochman, J. et al. (1973) Biochemistry 12:1130-1135; Sharon, J. et al. (1976) Biochemistry 15:1591-1594).). These various fragments are to be produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography (see Zola et al., supra).

The immunogen used to produce the present anti-Tpm antibodies may comprise the complete Tpm protein, or fragments or derivatives thereof. Preferred immunogens comprise all or a part of the AALBP central domain of Tpm. Immunogens comprising this domain are produced in a variety of ways known in the art, e.g., expression of cloned genes using conventional recombinant methods, isolation from cells of origin, cell populations expressing high levels of Tpm, etc.

The mAbs may be produced using conventional hybridoma technology, such as the procedures introduced by Kohler and Milstein (supra) and modifications thereof (see above references). An animal, preferably a mouse is primed by immunization with an immunogen as above to elicit the desired antibody response in the primed animal.

B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g., by immunoassay techniques using the Tpm protein. Positive clones are subcloned, e.g., by limiting dilution, and the mAbs are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single mAb can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al,. J. Biol. Chem. 1994 269:26267-26273; U.S. Pat. Nos. 4,704,692, 4,853,871, 4,94,6778, 5,260,203, 5,455,030). DNA sequences encoding the V regions of the H chain and the L chain are ligated to a linker encoding at least about 4 amino acids (typically small neutral amino acids). The protein encoded by this fusion allows assembly of a functional variable region that retains the specificity and affinity of the original antibody.

For in vivo use, particularly for injection into humans, it is desirable to decrease the immunogenicity of the mAb by humanizing the antibodies using methods known in the art. The humanized antibody may be the product of an animal having transgenic human Ig Constant region genes (see for example WO 90/10077 and WO 90/04036). Alternatively, the antibody of interest may be genetically engineered to substitute the CH₁, CH₂, CH₃, hinge domains, and/or the framework domain with the corresponding human sequence (see WO 92/02190).

Antibodies can be selected for particular desired properties. In the case of an antibody to be used for therapy, antibody screening procedures can include any of the in vitro or in vivo bioassays that measure angiogenesis, cell invasion, and the like. Moreover, the antibodies may be screened in various of the tumor models described herein to see if they promote or inhibit angiogenesis (or resultant tumor growth or metastasis). In this way, antibodies that are Tpm mimics or antagonists can be selected. Thus, the present invention includes therapeutic antibodies (discussed in more detail below) that promote angiogenesis by binding to and otherwise inhibiting the action of an antiangiogenic ligand at Tpm or its AALBP “domain.”

Use of Antibodies to Detect Cell Surface or Free Tpm or a AALBP Fragment

Antibodies specific for an epitope of Tpm are useful in immunoassays to detect molecules containing these epitopes on the surface of a cell or in a body fluid or sample, preferably serum or plasma. Such antibodies would detect Tpm, or an epitope-bearing fragment of Tpm. Thus, if proteolysis in the tumor milieu results in release of Tpm or the AALBP in plasma or in tissue.

By measuring the levels of released Tpm from tumor cells or activated ECs, the antibodies and immunoassays of this invention are used diagnostically to monitor the progress of a disease, where Tpm levels may reflect the amount of tumor tissue present.

Any conventional immunoassay known in the art may be employed for this purpose, though Enzyme Immunoassays such as ELISA are preferred. Immunoassay methods are also described in Coligan, J E et al., eds., Current Protocols in Immunology, Wiley-Interscience, New York 1991(or current edition); Butt, W. R. (ed.) Practical Immunoassay: The State of the Art, Dekker, New York, 1984; Bizollon, Ch. A., ed., Monoclonal Antibodies and New Trends in Immunoassays, Elsevier, N.Y., 1984; Butler, J. E., ELISA (Chapter 29), In: van Oss, C J et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T S et al., Laboratory Techniques and Biochemistry in Molecular Biology, North Holland Publishing Company, NY, (1978) (Chapter by Chard, T., “An Introduction to Radioimmune Assay and Related Techniques”).

In Vitro Testing of Compositions

A. Binding to Immobilized Tpm in a 96 Well Plate.

Binding to immobilized Tpm is carried out either by a competition assay with a known ligand, biotin-HKa, or by direct binding of the corresponding biotinylated protein. Plates are coated at room temperature with chicken gizzard Tpm (Sigma) in Tris buffer-saline (TBS) (200 ng/well). After incubation for 2 hours, wells are washed with TBS, then 1% BSA/TBS/Tween-20 is added to each well and incubate at 37° C. for two hours. HKa (Enzyme Laboratories) that had been previously biotinylated with EZ-Link (Pierce) according to the manufacturers instructions, is added to the plate at a concentration of 10 nM together with ZnCl₂ (10 μM) and the appropriate concentration of the protein, domain or peptide competitor. The plates are incubated at room temperature and then washed with TBS/Tween-20. Avidin-HRP is added, incubated for 20 minutes at room temperature, washed with TBS/Tween-20 and the chromogenic substrate is added. The reaction is stopped with sulfuric acid and the plate read at 490 nm.

B. Assay for EC Migration

For EC migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as HUVEC, which have been detached from monolayer culture using trypsin, are diluted to a final concentration of about 10⁶ cells/mL with serum-free media and 0.2 mL of this cell suspension is added to the upper chamber of each transwell. Inhibitors to be tested are added to both the upper and lower chambers, and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated.

C. Biological Assay of Anti-Invasive Activity

The compositions of the invention are tested for their anti-invasive capacity. The ability of cells such as ECs or tumor cells (e.g., PC-3 human prostatic carcinoma) cells to invade through a reconstituted basement membrane (Matrigel®) in an assay known as a Matrigel® invasion assay system as described in detail by Kleinman et al., Biochemistry 25: 312-318,1986 and Parish et al., Int. J. Cancer 52:378-383,1992. Matrigel® is a reconstituted basement membrane containing type IV collagen, laminin, heparan sulfate proteoglycans such as perlecan, which bind to and localize bFGF, vitronectin as well as transforming growth factor-β (TGFβ), urokinase-type plasminogen activator (uPA), tissue plasminogen activator (tPA), and the serpin known as plasminogen activator inhibitor type 1 (PAI-1) (Chambers et al., Canc. Res. 55:1578-1585, 1995). It is accepted in the art that results obtained in this assay for compounds which target extracellular receptors or enzymes are predictive of the efficacy of these compounds in vivo (Rabbani et al., Int. J. Cancer 63: 840-845, 1995).

Such assays employ transwell tissue culture inserts. Invasive cells are defined as cells which are able to traverse through the Matrigel® and upper aspect of a polycarbonate membrane and adhere to the bottom of the membrane. Transwells (Costar) containing polycarbonate membranes (8.0 μm pore size) are coated with Matrigel® (Collaborative Research), which has been diluted in sterile PBS to a final concentration of 75 μg/mL (60 μL of diluted Matrigel® per insert), and placed in the wells of a 24-well plate. The membranes are dried overnight in a biological safety cabinet, then rehydrated by adding 100 μL of DMEM containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DiffQuik® (Giemsa stain) and the upper chamber is then scraped using a cotton swab to remove the Matrigel® and any cells which did not invade through the membrane. The membranes are detached from the transwell using an X-acto® blade, mounted on slides using Permount® and cover-slips, then counted under a high-powered (400×) field. An average of the cells invaded is determined from 5-10 fields counted and plotted as a function of inhibitor concentration.

D. Tube-Formation Assays of Anti-Angiogenic Activity

The compounds of this invention are tested for their anti-angiogenic activity in one of two different assay systems in vitro.

ECs, for example, HUVEC or human microvascular ECs (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch).

This assay is recognized in the art to be predictive of angiogenic (or anti-angiogenic) efficacy in vivo (Min, H Y et al., Cancer Res. 56: 2428-2433,1996).

In an alternate assay, EC tube formation is observed when ECs are cultured on Matrigel® (Schnaper et al., J. Cell. Physiol. 165:107-118 1995). ECs(1×10⁴ cells/well) are transferred onto Matrigel®-coated 24-well plates, and tube formation is quantitated after 48 hrs. Inhibitors are tested by adding them either at the same time as the ECs or at various time points thereafter. Tube formation can also be stimulated by adding (a) angiogenic growth factors such as bFGF or VEGF, (b) differentiation stimulating agents (e.g.,. PMA) or (c) a combination of these.

This assay models angiogenesis by presenting to the ECs a particular type of basement membrane, namely the layer of matrix which migrating and differentiating ECs might be expected to first encounter. In addition to bound growth factors, the matrix components found in Matrigel® (and in basement membranes in situ) or proteolytic products thereof may also be stimulatory for EC tube formation which makes this model complementary to the fibrin gel angiogenesis model previously described (Blood et al., Biochim. Biophys. Acta 1032:89-118, 1990; Odedra et al., Pharmac. Ther. 49:111-124, 1991). The compounds of this invention inhibit EC tube formation in both assays, which suggests that the compounds will also have anti-angiogenic activity.

E. Assays for the Inhibition of Proliferation

The ability of the compounds of the invention to inhibit the proliferation of EC's may be determined in a 96-well format. Type I collagen (gelatin) is used to coat the wells of the plate (0.1-1 mg/mL in PBS, 0.1 mL per well for 30 minutes at room temperature). After washing the plate (3×w/PBS), 3-6,000 cells are plated per well and allowed to attach for 4 hrs (37° C./5% CO₂) in Endothelial Growth Medium MGM; Clonetics) or M199 media containing 0.1-2% FBS. The media and any unattached cells are removed at the end of 4 hrs and fresh media containing bFGF (1-10 ng/L) or VEGF (1-10 ng/mL) is added to each well. Compounds to be tested are added last and the plate is allowed to incubate (37° C./5% CO₂) for 24-48 hrs. MTS (Promega) is added to each well and allowed to incubate from 1-4 hrs. The absorbance at 490 nm, which is proportional to the cell number, is then measured to determine the differences in proliferation between control wells and those containing test compounds.

A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Compounds to be tested are included in some of the wells. After 24 hrs, MTS is added to the plate and the assay developed and read as described above.

F. Assays of Cytotoxicity

The anti-proliferative and cytotoxic effects of the compositions may be determined for various cell types including tumor cells, ECs, fibroblasts and macrophages. This is especially useful when testing a compound of the invention which has been conjugated to a therapeutic moiety such as a radiotherapeutic or a toxin. For example, a conjugate of one of the compositions with Bolton-Hunter reagent which has been iodinated with ¹³¹I, would be expected to inhibit the proliferation of cells expressing surface Tpm or AALBP (most likely by inducing apoptosis). Anti-proliferative effects would be expected against tumor cells and stimulated ECs but, under some circumstances not quiescent ECs or normal human dermal fibroblasts. Any anti-proliferative or cytotoxic effects observed in the normal cells would represent non-specific toxicity of the conjugate.

A typical assay would involve plating cells at a density of 5-10,000 cells per well in a 96-well plate. The compound to be tested is added at a concentration 10× the IC₅₀ measured in a binding assay (this will vary depending on the conjugate) and allowed to incubate with the cells for 30 minutes. The cells are washed 3× with media, then fresh media containing [³H]thymidine (1 μCi/mL) is added to the cells and they are allowed to incubate at 37° C. in 5% CO₂ for 24 and 48 hours. Cells are lysed at the various time points using 1 M NaOH and counts per well determined using a β-counter. Proliferation may be measured non-radioactively using MTS reagent or CyQuant® to measure total cell number. For cytotoxicity assays (measuring cell lysis), a Promega 96-well cytotoxicity kit is used. If there is evidence of anti-proliferative activity, induction of apoptosis may be measured using TumorTACS (Genzyme).

G. Caspase-3 Activity

The ability of the compounds of the invention to promote apoptosis of EC's may be determined by measuring activation of caspase-3. Type I collagen (gelatin) is used to coat a P100 plate and 5×10⁵ ECs are seeded in EGM containing 10% FBS. After 24 hours (at 37° C. in 5% CO₂) the medium is replaced by EGM containing 2% FBS, 10 ng/ml bFGF and the desired test compound. The cells are harvested after 6 hours, cell lysates prepared in 1% Triton and assayed using the EnzChek®Caspase-3 Assay Kit #1 (Molecular Probes) according to the manufactures' instructions.

In Vivo Study of Tpm-Binding Antiangiogenic Polypeptides or Peptides

A. Corneal Angiogenesis Model

The protocol used is essentially identical to that described by Volpert et al. (J. Clin. Invest. 98:671-679 (1996)). Briefly, female Fischer rats (120-140 gms) are anesthetized and pellets (5 μl) comprised of Hydron®, bFGF (150 nM), and the compounds to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

B. Matrigel® Plug Assay

This assay is performed essentially as described by Passaniti et al. (Lab Invest. 67:519-528 (1992). Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-EC adhesion molecule or PECAM”) may also be used to confirm neovessel formation and microvessel density in the plugs.

C. Chick Chorioallantoic Membrane (CAM) Angiogenesis Assay

This assay is performed essentially as described by Nguyen et al (Microvascular Res. 47:31-40 (1994)). A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

D. In Vivo Assessment Angiogenesis Inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel® and then injected into the flank of a mouse following the protocol described in Sec. B., above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenize with a tissue homogenizer.

E. Xenograft Model of Subcutaneous (s.c.) Tumor Growth

Nude mice are inoculated with MDA-MB-231 cells (human breast carcinoma) and Matrigel® (1×10⁶ cells in 0.2 mL) s.c. in the right flank of the animals. The tumors are staged to 200 mm³ and then treatment with a test composition is initiated (100 μg/animal/day given q.d. IP). Tumor volumes are obtained every other day and the animals are sacrificed after 2 weeks of treatment. The tumors are excised, weighed and paraffin embedded. Histological sections of the tumors are analyzed by H and E, anti-CD31, Ki-67, TUNEL, and CD68 staining.

F. Xenograft Model of Metastasis

The compounds of this invention are also tested for inhibition of late metastasis using an experimental metastasis model (Crowley, C W et al., Proc. Natl. Acad. Sci. USA 90 5021-5025 (1993)). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This approach permits utilization of either of these markers (fluorescence detection of GFP or histochemical colorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected iv into the tail veins of nude (nu/nu) mice. Animals are treated with a test composition at 100 μg/animal/day given q.d. IP. Single metastatic cells and foci are visualized and quantitated by fluorescence microscopy or light microscopic histochemistry or by grinding the tissue and quantitative colorimetric assay of the detectable label.

G. Inhibition of Spontaneous Metastasis In Vivo by Antiangiogenic Agents Acting at Cell Surface Tropomyosin

The rat syngeneic breast cancer system (Xing et al., Int. J Cancer 67:423-429 (1996) employs Mat BIII rat breast cancer cells. Tumor cells, for example about 10⁶ suspended in 0.1 mL PBS, are inoculated into the mammary fat pads of female Fisher rats. At the time of inoculation, a 14-day Alza osmotic mini-pump is implanted intraperitoneally to dispense the test compound. The compound is dissolved in PBS (e.g., 200 mM stock), sterile filtered and placed in the minipump to achieve a release rate of about 4 mg/kg/day. Control animals receive vehicle (PBS) alone or a vehicle control peptide in the minipump. Animals are sacrificed at about day 14.

Therapeutic Outcomes

In the rats treated with the active compounds of the present invention, significant reductions in the size of the primary tumor and in the number of metastases in the spleen, lungs, liver, kidney and lymph nodes (enumerated as discrete foci) are observed. Histological and immunohistochemical analysis reveal increased necrosis and signs of apoptosis in tumors in treated animals. Large necrotic areas are seen in tumor regions lacking neovascularization. Human or rabbit HPRG or HKa or the D5 domain and their derivatives to which ¹³¹I is conjugated (either 1 or 2 I atoms per molecule of peptide) are effective radiotherapeutics and are found to be at least two-fold more potent than the unconjugated polypeptides. In contrast, treatment with control peptides fails to cause a significant change in tumor size or metastasis.

H. 3LL Lewis Lung Carcinoma: Primary Tumor Growth

This tumor line arose spontaneously in 1951 as carcinoma of the lung in a C57BL/6 mouse (Cancer Res 15:39, 1955. See, also Malave, I et al., J. Nat'l. Canc. Inst. 62:83-88 (1979)). It is propogated by passage in C57BL/6 mice by subcutaneous (sc) inoculation and is tested in semiallogeneic C57BL/6×DBA/2 F₁ mice or in allogeneic C3H mice. Typically six animals per group for subcutaneously (sc) implant, or ten for intramuscular (im) implant are used. Tumor may be implanted sc as a 2-4 mm fragment, or im or sc as an inoculum of suspended cells of about 0.5-2×10⁶-cells. Treatment begins 24 hours after implant or is delayed until a tumor of specified size (usually approximately 400 mg) can be palpated. The test compound is administered ip daily for 11 days Animals are followed by weighing, palpation, and measurement of tumor size. Typical tumor weight in untreated control recipients on day 12 after im inoculation is 500-2500 mg. Typical median survival time is 18-28 days. A positive control compound, for example cyclophosphamide at 20 mg/kg/injection per day on days 1-11 is used. Results computed include mean animal weight, tumor size, tumor weight, survival time. For confirmed therapeutic activity, the test composition should be tested in two multi-dose assays.

I. 3LL Lewis Lung Carcinoma: Primary Growth and Metastasis Model

This model has been utilized by a number of investigators. See, for example, Gorelik, E et al., J. Nat'l. Canc. Inst. 65:1257-1264 (1980); Gorelik, E. et al., Rec. Results Canc. Res. 75:20-28 (1980); Isakov, N et al., Invasion Metas. 2:12-32 (1982); Talmadge J E et al., J. Nat'l Canc. Inst. 69:975-980 (1982); Hilgard, P. et al., Br. J. Cancer 35:78-86(1977)). Test mice are male C57BL/6 mice, 2-3 months old. Following sc, im, or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725).

Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 3-4 days after dorsal sc injection of 10⁶ cells. The day of tumor appearance and the diameters of established tumors are measured by caliper every two days.

The treatment is given as one or two doses of peptide or derivative, per week. In another embodiment, the peptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M. L. et al., J. Lab. Clin. Med. 89:217-228 (1977). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd (iododeoxyuridine). One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints; or (2) mice are left intact as nonamputated tumor-bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

For a compound to be useful in accordance with this invention, it should demonstrate activity in at least one of the above (in vitro or in vivo) assay systems.

Pharmaceutical and Therapeutic Compositions and Their Administration

The compounds that may be employed in the pharmaceutical compositions of the invention include all of the polypeptide and peptide compounds described above, as well as the pharmaceutically acceptable salts of these compounds. Pharmaceutically acceptable acid addition salts of the compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts.

Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

As stated above, the compounds of the invention possess the ability to inhibit EC proliferation, motility, or invasiveness and angiogenesis, properties that are exploited in the treatment of cancer, in particular metastatic cancer. A composition of this invention may be active per se, or may act as a “pro-drug” that is converted in vivo to the active form.

Therapeutically Labeled Compositions

In a preferred embodiment, the polypeptide and peptides describe herein are “therapeutically conjugated” or “therapeutically labeled” (terms which are intended to be interchangeable) and used to deliver a therapeutic agent to the site to which the compounds home and bind, such as sites of tumor metastasis or foci of infection/inflammation, restenosis or fibrosis. The term “therapeutically conjugated” means that the modified peptide is conjugated to another therapeutic agent that is directed either to the underlying cause or to a “component” of tumor invasion, angiogenesis, inflammation or other pathology. A therapeutically labeled protein or peptide carries a suitable therapeutic “label” also referred to herein as a “therapeutic moiety.” A therapeutic moiety is an atom, a molecule, a compound or any chemical component added to the peptide that renders it active in treating a target disease or condition, primarily one a associated with undesired angiogenesis. As noted above, the peptides of the present invention are prepared by conventional means, either chemical synthesis, proteolysis of Tpm or its antiangiogenic ligands or recombinant means. The therapeutic moiety may be bound directly or indirectly to the peptide. The therapeutically labeled protein or peptide is administered as pharmaceutical composition which comprises a pharmaceutically acceptable carrier or excipient, and is preferably in a form suitable for injection.

Examples of useful therapeutic radioisotopes (ordered by atomic number) include ⁴⁷Sc, ⁶⁷Cu, ⁹⁰Y, ¹⁰⁹Pd, ¹²⁵I, ¹³¹I, ¹⁸⁶Re, ¹⁸⁸Re, 199 Au, ²¹¹At, ²¹²Pb and ²¹⁷Bi. These atoms can be conjugated to the peptide directly, indirectly as part of a chelate, or, in the case of iodine, indirectly as part of an iodinated Bolton-Hunter group. The radioiodine can be introduced either before or after this group is coupled to the peptide compound.

Preferred doses of the radionuclide conjugates are a function of the specific radioactivity to be delivered to the target site which varies with tumor type, tumor location and vascularization, kinetics and biodistribution of the peptide carrier, energy of radioactive emission by the nuclide, etc. Those skilled in the art of radiotherapy can readily adjust the dose of the peptide in conjunction with the dose of the particular nuclide to effect the desired therapeutic benefit without undue experimentation.

Another therapeutic approach included here is the use of boron neutron capture therapy, where a boronated peptide is delivered to a desired target site, such as a tumor, most preferably an intracranial tumor (Barth, R F, Cancer Invest. 14:534-550 (1996); Mishima, Y. (ed.), Cancer Neutron Capture Therapy, New York: Plenum Publishing Corp., 1996; Soloway, A. H., et al., (eds), J. Neuro-Oncol. 33:1-188 (1997). The stable isotope ¹⁰B is irradiated with low energy (<0.025 eV) thermal neutrons, and the resulting nuclear capture yields α-particles and ⁷Li nuclei which have high linear energy transfer and respective path lengths of about 9 and 5 μm. This method is predicated on ¹⁰B accumulation in the tumor with lower levels in blood, ECs and normal tissue (e.g., brain). Such delivery has been accomplished using epidermal growth factor (Yang. W. et al., Cancer Res 57:4333-4339 (1997).

Other therapeutic agents which can be coupled to the peptide compounds according to the method of the invention are drugs, prodrugs, enzymes for activating pro-drugs, photosensitizing agents, nucleic acid therapeutics, antisense vectors, viral vectors, lectins and other toxins.

Lectins are proteins, commonly derived from plants, that bind to carbohydrates. Among other activities, some lectins are toxic. Some of the most cytotoxic substances known are protein toxins of bacterial and plant origin (Frankel, A E, et al., Ann. Rev. Med. 37:125-142 (1986)). These molecules binding the cell surface and inhibit cellular protein synthesis. The most commonly used plant toxins are ricin and abrin; the most commonly used bacterial toxins are diphtheria toxin and Pseudomonas exotoxin A. In ricin and abrin, the binding and toxic functions are contained in two separate protein subunits, the A and B chains. The ricin B chain binds to the cell surface carbohydrates and promotes the uptake of the A chain into the cell. Once inside the cell, the ricin A chain inhibits protein synthesis by inactivating the 60S subunit of the eukaryotic ribosome Endo, Y. et al., J. Biol. Chem. 262: 5908-5912 (1987)). Other plant derived toxins, which are single chain ribosomal inhibitory proteins, include pokeweed antiviral protein, wheat germ protein, gelonin, dianthins, momorcharins, trichosanthin, and many others (Strip, F. et al., FEBS Lett. 195:1-8 (1986)). Diphtheria toxin and Pseudomonas exotoxin A are also single chain proteins, and their binding and toxicity functions reside in separate domains of the same protein Pseudomonas exotoxin A has the same catalytic activity as diphtheria toxin. Ricin has been used therapeutically by binding its toxic α-chain, to targeting molecules such as antibodies to enable site-specific delivery of the toxic effect. Bacterial toxins have also been used as anti-tumor conjugates. As intended herein, a toxic peptide chain or domain is conjugated to a compound of this invention and delivered in a site-specific manner to a target site where the toxic activity is desired, such as a metastatic focus. Conjugation of toxins to protein such as antibodies or other ligands are known in the art (Olsnes, S. et al., Immunol. Today 10:291-295 (1989); Vitetta, E. S. et al., Ann. Rev. Immunol. 3:197-212 (1985)).

Cytotoxic drugs that interfere with critical cellular processes including DNA, RNA, and protein synthesis, have been conjugated to antibodies and subsequently used for in vivo therapy. Such drugs, including, but not limited to, daunorubicin, doxorubicin, methotrexate, and Mitomycin C are also coupled to the compounds of this invention and used therapeutically in this form.

In a preferred embodiment of this invention, a cytotoxic drug is targeted to Tpm on the surface of an EC or tumor cell by conjugating the drug to a Tpm ligand. Again, preferred ligands for cell surface Tpm include HPRG or a H/P domain peptide thereof, HKa or the D5 domain or a shorter fragment thereof, or more preferably, an antibody specific for an epitope of Tpm expressed on these cell surfaces.

The compounds of the invention, as well as the pharmaceutically acceptable salts thereof, may be incorporated into convenient dosage forms, such as capsules, impregnated wafers, tablets or injectable preparations. Solid or liquid pharmaceutically acceptable carriers may be employed.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pennsylvania (Gennaro 18th ed. 1990).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the diagnosis or treatment of any of a number of animal genera and species, and are equally applicable in the practice of human or veterinary medicine. Thus, the pharmaceutical compositions can be used to treat domestic and commercial animals, including birds and more preferably mammals, as well as humans.

The term “systemic administration” refers to administration of a composition or agent such as the polypeptide, peptides or nucleic acids described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial (or lung instillation), intracranial, intra-aural or intraocular. The term “local administration” refers to administration of a composition or drug into a limited, or circumscribed, anatomic space, such as intratumoral injection into a tumor mass, subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration. Injectables or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, e.g., as an ointment, cream or gel; orally; rectally; e.g., as a suppository.

For topical application, the compound may be incorporated into topically applied vehicles such as a salve or ointment. The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Preferred vehicles for non-sprayable topical preparations include ointment bases, e.g., polyethylene glycol-1000 (PEG-1000); conventional creams such as HEB cream; gels; as well as petroleum jelly and the like.

Also suitable for topic application as well as for lung instillation are sprayable aerosol preparations wherein the compound, preferably in combination with a solid or liquid inert carrier material, is packaged in a squeeze bottle or in admixture with a pressurized volatile, normally gaseous propellant. The aerosol preparations can contain solvents, buffers, surfactants, perfumes, and/or antioxidants in addition to the compounds of the invention.

For the preferred topical applications, especially for humans, it is preferred to administer an effective amount of the compound to an affected area, e.g., skin surface, mucous membrane, eyes, etc. This amount will generally range from about 0.001 mg to about 1 g per application, depending upon the area to be treated, the severity of the symptoms, and the nature of the topical vehicle employed.

Antiangiogenic compositions may be administered in combination with a biodegradable, biocompatible polymeric implant which releases the troponin active agent over a controlled period of time at a selected site. Examples of preferred polymeric materials include polyanhydrides, polyorthoesters, polyglycolic acid, polylactic acid, polyethylene vinyl acetate, and copolymers and blends thereof. See, for example, Medical Applications of Controlled Release, Langer and Wise (eds.), 1974, CRC Press, Boca Raton, Fla.; Controlled Drug Bioavailability, Drug Product Design and Performance, Smolen and Ball (eds.), 1984, Wiley, NY; Ranger et al., 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105. In another embodiment, a controlled release system can be placed in proximity of the therapeutic target, e.g., the brain, thus requiring only a fraction of the systemic dose (Goodson, In: Medical Applications of Controlled Release, supra, vol. 2, pp. 115-138). Other controlled release systems are discussed in a review by Langer, R, 1990, Science 249:1527-1533).

Other pharmaceutically acceptable carriers for polypeptide or nucleic acid compositions of the present invention are liposomes, pharmaceutical compositions in which the active protein is contained either dispersed or variously present in corpuscles consisting of aqueous concentric layers adherent to lipidic layers. The active polypeptide or peptide, or the nucleic acid is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Therapeutic compositions for treating tumors and cancer may comprise, in addition to the antiangiogenic polypeptide or peptide, one or more additional anti-tumor agents, such as mitotic inhibitors, e.g., vinblastine; alkylating agents, e.g., cyclophosphamide; folate inhibitors, e.g., methotrexate, piritrexim or trimetrexate; antimetabolites, e.g., 5-fluorouracil and cytosine arabinoside; intercalating antibiotics, e.g., adriamycin and bleomycin; enzymes or enzyme inhibitors, e.g., asparaginase, topoisomerase inhibitors such as etoposide; or biological response modifiers, e.g., interferons or interleukins. In fact, pharmaceutical compositions comprising any known cancer therapeutic in combination with the peptides disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

While some antibodies will bind directly to cell surface Tpm and induce antiangiogenic activity (“agonists”) other antibodies specific for epitopes of Tpm are expected to inhibit binding of and anti-angiogenic effects of HPRG via the H/P domain, D5, etc. Such antibodies, termed “antagonists” are useful in the induction of neovascularization and can be used to treat diseases or conditions in which increased angiogenesis is desired. Such conditions include coronary artery disease and peripheral artery disease, in which therapeutic angiogenesis is know to be beneficial (Freedman S B et al., Ann Intern Med, 2002, 136:54-71 and J Mol Cell Cardiol, 2001 33:379-393; Durairaj, A. et al, Cardiol Rev, 2000, 8:279-287; Emanueli, C et al., Br J Pharmcol, 2001, 133:951-958; Isner, J M et al., Hum Gene Ther, 1996, 7:959-88). In general, any form of tissue ischemia resulting from vascular occlusion, vascular disease or surgery can be treated in this manner (Isner et al., supra; Webster K A., Crit Rev Eukaryot Gene Expr, 2000, 10:113-125), for example peripheral limb ischemia or hepatic arterial occlusion in liver transplantation (Yedlicka, J W et al., J Vasc Interv Radiol, 1991, 2:235-240) where the present antibodies will promote revascularization of ischemic tissues.

These antagonist antibodies are useful in the promotion of wound healing (including recovery from surgical wounds), which is known to be dependent upon angiogenic processes (Liekens S et al., Biochem Pharmacol, 2001, 61:253-270; Lingen, M W, Arch Pathol Lab Med, 2001, 125:67-71; Raza S L et al., J Investig Dermatol Symp Proc, 2000, 5:47-54; Tonnesen M G et al., J Investig Dermatol Symp Proc, 2000, 5:40-46; Hunt T K, Adv Skin Wound Care, 2000, 13(2 Suppl):6-11; Grant D S et al., Adv Exp Med Biol, 2000, 476:139-154; Drixler T A et al., Eur J Surg, 2000, 166:435-446; Singer A J et al., N Engl J Med, 1999, 341:738-746; Martin, P, Science, 1997, 276:75-81) and in accelerating or enhancing fracture repair (Glowacki, J, Clin Orthop, 1998, 355 Suppl:S82-89).

Antagonist anti-Tpm antibodies can be used in conjunction with cellular therapy and transplantation of pancreatic islet cells in the treatment of diabetes as vascular endothelium acts to stimulate or induce pancreatic organogenesis and insulin production by pancreatic beta cells (Lammert E et al., Science, 2001, 294:564-567; see also page 530-531). Liver organogenesis is also promoted by vasculogenic ECs and nascent vessels (Matsumoto, K. et al., Science, 2001, 294:559-563). See also, DeFrancesco, L., The Scientist 15:17 (2001).

Screening of antibodies or supernatants of hybridoma cultures to detect anti-Tpm antibodies with the desired antiangiogenic or pro-angiogenic activity are performed using the in vitro and in vivo bioassays described above, such as the Matrigel® plug assay.

Therapeutic Methods

The methods of this invention may be used to inhibit tumor growth and invasion in a subject or to suppress angiogenesis induced by tumors by inhibiting EC growth and migration. By inhibiting the growth or invasion of a tumor or angiogenesis, the methods result in inhibition of tumor metastasis. A vertebrate subject, preferably a mammal, more preferably a human, is administered an amount of the compound effective to inhibit tumor growth, invasion or angiogenesis. The compound or pharmaceutically acceptable salt thereof is preferably administered in the form of a pharmaceutical composition as described above.

Doses of the proteins (including antibodies), peptides, peptide multimers, etc., preferably include pharmaceutical dosage units comprising an effective amount of the peptide. Dosage unit form refers to physically discrete units suited as unitary dosages for a mammalian subject; each unit contains a predetermined quantity of active material (e.g., the HPRG-derived domain or peptide, or nucleic acid encoding the polypeptide) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects.

By an effective amount is meant an amount sufficient to achieve a steady state concentration in vivo which results in a measurable reduction in any relevant parameter of disease and may include growth of primary or metastatic tumor, any accepted index of inflammatory reactivity, or a measurable prolongation of disease-free interval or of survival. For example, a reduction in tumor growth in 20% of patients is considered efficacious (Frei III, E., The Cancer Journal 3:127-136 (1997)). However, an effect of this magnitude is not considered to be a minimal requirement for the dose to be effective in accordance with this invention.

In one embodiment, an effective dose is preferably 10-fold and more preferably 100-fold higher than the 50% effective dose (ED₅₀) of the compound in an in vivo assay as described herein.

The amount of active compound to be administered depends on the precise peptide or derivative selected, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

The therapeutically effective dosage for inhibition of angiogenesis in vivo, which include any one of more of inhibition of capillary endothelial cell proliferation, migration, and blood vessel ingrowth, may be extrapolated from in vitro inhibition assays described herein The effective dosage is also dependent on the method and means of delivery. For example, in some applications, as in the treatment of psoriasis or diabetic retinopathy, the inhibitor is delivered in a topical-ophthalmic carrier. In other applications, as in the treatment of solid tumors, the inhibitor is delivered by means of a biodegradable, polymeric implant. The protein can also be modified, for example, by polyethylene glycol treatment which would affect the effective dose.

A preferred dose of an agonist anti-Tpm for treating a subject, preferably mammalian, more preferably human, with a tumor is an amount of up to about 100 milligrams of active protein or peptide-based compound per kilogram of body weight. A typical single dosage of the peptide or peptidomimetic is between about 1 ng and about 100 mg/kg body weight. For topical administration, dosages in the range of about 0.01-20% concentration (by weight) of the compound, preferably 1-5%, are suggested. A total daily dosage in the range of about 0.1 milligrams to about 7 grams is preferred for intravenous administration. The foregoing ranges are, however, suggestive, as the number of variables in an individual treatment regime is large, and considerable excursions from these preferred values are expected.

An effective amount or dose of the peptide for inhibiting EC proliferation or migration in vitro is in the range of about 1 picogram to about 5 nanograms per cell. Effective doses and optimal dose ranges may be determined in vitro using the methods described herein.

The compounds of the invention may be characterized as producing an inhibitory effect on tumor cell or EC proliferation, migration, invasion, or on angiogenesis, on tumor metastasis or on inflammatory reactions. The compounds are especially useful in producing an anti-tumor effect in a mammalian host, preferably human, harboring a tumor.

Angiogenesis inhibitors may play a role in preventing inflammatory angiogenesis and gliosis following traumatic spinal cord injury, thereby promoting the reestablishment of neuronal connectivity (Wamil, A W et al., Proc. Nat'l. Acad. Sci. USA 95:13188-13193 (1998)). Therefore, the compositions of the present invention are administered as soon as possible after traumatic spinal cord injury and for several days up to about two weeks thereafter to inhibit the angiogenesis and gliosis that would sterically prevent reestablishment of neuronal connectivity. The treatment reduces the area of damage at the site of spinal cord injury and facilitates regeneration of neuronal function and thereby prevents paralysis. The compounds of the invention are expected also to protect axons from Wallerian degeneration, reverse aminobutyrate-mediated depolarization (occurring in traumatized neurons), and improve recovery of neuronal conductivity of isolated central nervous system cells and tissue in culture.

General Recombinant DNA Methods

Basic texts disclosing general methods of molecular biology, all of which are incorporated by reference, include: Sambrook, J et al., Molecular Cloning: A Laboratory Manual, 2nd (or later) Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, editor, DNA Cloning: A Practical Approach, vol. I & II, II Press, 1985; Albers, B. et al., Molecular Biology of the Cell, 2nd (or later) Ed., Garland Publishing, Inc., New York, N.Y. (1989); Watson, J D et al., Recombinant DNA, 2nd (or later) Ed., Scientific American Books, New York, 1992; and Old, R W et al., Principles of Gene Manipulation: An Introduction to Genetic Engineering, 2nd (or later) Ed., University of California Press, Berkeley, Calif. (1981).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, cDNA molecules encoding the amino acid sequence corresponding to the Tpm polypeptide, domain or peptide fragment of the present invention, or active variants thereof, can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

This invention includes isolated nucleic acids having a nucleotide sequence encoding the novel Tpm polypeptide, domain, peptide fragment, or equivalent thereof, and their use in transfecting cells in vitro or in vivo to express their polypeptide product. The term nucleic acid as used herein is intended to include such fragments or equivalents. The nucleic acid sequences of this invention can be DNA or RNA.

A cDNA nucleotide sequence encoding a Tpm polypeptide can be obtained by isolating total mRNA from an appropriate cell line. Double stranded cDNA is prepared from total mRNA cDNA can be inserted into a suitable plasmid, bacteriophage or viral vector using any one of a number of known techniques.

In reference to a nucleotide sequence, the term “equivalent” is intended to include sequences encoding structurally homologous and/or a functionally equivalent proteins such as naturally occurring isoforms or related, immunologically cross-reactive family members of these proteins. Such isoforms or family members are defined as proteins that share function and amino acid sequence similarity to, for example, SEQ ID NO:1 or 3 or SEQ ID NO:5, 7, 9, 11, 13,15 17 or 19.

Fragments of Nucleic Acid

A fragment of the nucleic acid sequence is defined as a nucleotide sequence having fewer nucleotides than the nucleotide sequence encoding the full length Tpm protein, the AALBP fragment or smaller fragments or domains. This invention includes such nucleic acid fragments that encode polypeptides which retain (1) the ability of the Tpm polypeptide to bind an inhibitor of angiogenesis, endothelial tube formation, cell invasion or tumor growth or metastasis.

Generally, the nucleic acid sequence encoding a fragment of Tpm comprises of nucleotides from the sequence encoding the mature protein (or the active fragment thereof).

Nucleic acid sequences, particularly those that encode peptide multimers of this invention may also include linker or spacer sequences (preferably encoding Gly₁₋₆). The nucleic acids further may include natural or modified restriction endonuclease sites and other sequences that are useful for manipulations related to cloning, expression or purification of encoded polypeptide or peptides. These and other modifications of nucleic acid sequences are described herein or are well-known in the art.

The techniques for assembling and expressing DNA coding sequences include synthesis of oligonucleotides, PCR, transforming cells, constructing vectors, expression systems, and the like; these are well-established in the art such that those of ordinary skill are familiar with standard resource materials, specific conditions and procedures.

Expression Vectors and Host Cells

This invention includes an expression vector comprising a nucleic acid sequence encoding a Tpm polypeptide, domain, or peptide operably linked to at least one regulatory sequence.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA, e.g., plasmids, viruses, and the like U.S. Pat. No. 5,217,879), and includes both the expression and nonexpression plasmids. Where a recombinant microorganism or cell culture is a host for an “expression vector,” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and the nature (e.g., size) of the polypeptide to be expressed.

The present expression vectors comprise the full range of nucleic acid molecules encoding the various embodiments of the Tpm polypeptide, fragment or peptide.

Such expression vectors are used to transfect host cells (in vitro, ex vivo or in vivo) for expression of the DNA and production of the encoded proteins which include fusion proteins or peptides. It will be understood that a genetically modified cell expressing the Tpm polypeptide, domain, peptide fragment or multimer, may transiently express the exogenous DNA for a time sufficient for the cell to be useful for its stated purpose.

Host cells may also be transfected with one or more expression vectors that singly or in combination comprise DNA encoding at least a portion of the Tpm polypeptide or AALBP fragment or shorter peptide and DNA encoding at least a portion of a second Tpm-derived sequence (or variant), so that the host cells produce yet further Tpm polypeptide, domain or peptide fragments that include both the portions.

Methods for producing the Tpm polypeptide, domain or peptide fragments, are all conventional in the art. Cultures typically includes host cells, appropriate growth media and other byproducts. Suitable culture media are well known in the art. The Tpm polypeptide, domain or peptide fragment can be isolated from medium or cell lysates using conventional techniques for purifying proteins and peptides, including ammonium sulfate precipitation, fractionation column chromatography (e.g. ion exchange, gel filtration, affinity chromatography, etc.) and/or electrophoresis (see generally, Meth Enzymol, 22:233-577 (1971)). Once purified, partially or to homogeneity, the recombinant polypeptides of the invention can be utilized in pharmaceutical compositions as described in more detail herein.

The term “isolated” as used herein, when referring to a molecule or composition, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). It is understood that even where a protein has been isolated so as to appear as a homogenous or dominant band in a gel pattern, there are generally trace contaminants which co-purify with it.

Prokaryotic or eukaryotic host cells transformed or transfected to express the Tpm polypeptide, domain or peptide fragment are within the scope of the invention. For example, the Tpm polypeptide, domain or peptide fragment may be expressed in bacterial cells such as E. coli, insect cells (baculovirus), yeast, or mammalian cells such as Chinese hamster ovary cells (CHO) or human cells (which are preferred for human therapeutic use of the transfected cells). Other suitable host cells may be found in Goeddel, (1990) supra or are otherwise known to those skilled in the art.

Expression in eukaryotic cells leads to partial or complete glycosylation and/or formation of relevant inter- or intra-chain disulfide bonds of the recombinant polypeptide.

Examples of vectors for expression in yeast S. cerevisiae include pYepSec1 (Baidari et al., (1987) EMBO J: 6:229-234), pMFa (Kujan et al. (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165,) and the pVL series (Lucklow, V. A., and Summers, M. D., (1989) Virology 170:31-39). Generally, COS cells (Gluzman, Y., (1981) Cell 23:175-182) are used in conjunction with such vectors as pCDM8 (Aruffo A. and Seed, B., supra, for transient amplification/expression in mammalian cells, while CHO (dhfr-negative CHO) cells are used with vectors such as pMT2PC (Kaufman et al. (1987), EMBO J. 6:187-195) for stable amplification/expression in mammalian cells. The NS0 myeloma cell line (a glutamine synthetase expression system.) is available from Celltech Ltd.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant polypeptide.

Inducible non-fusion expression vectors include pTrc (Amann et al., (1988) Gene 69:301-315) and pET 1 d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lacO fusion promoter mediated by coexpressed viral RNA polymerase (T7gn1). Th is viral polymerase is supplied by host strains BL21(DE3) or HMS 174(DE3) from a resident λ prophage harboring a T7gn1 under the transcriptional control of the lacUV 5 promoter.

Vector Construction

Construction of suitable vectors containing the desired coding and control sequences employs standard ligation and restriction techniques which are well understood in the art. Isolated plasmids, DNA sequences, or synthesized oligonucleotides are cleaved, tailored, and re-ligated in the form desired. The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, for example, Edge, M. D., Nature (1981) 292:756; Nambair, K. P., et al., Science (1984) 223:1299; and Jay, E., J Biol Chem (1984) 259:6311.

Synthetic oligonucleotides are prepared by either the phosphotriester method as described by references cited above or the phosphoramidite method as described by Beaucage, S. L., and Caruthers, M. H., Tetrahed Lett (1981) 22:1859; and Matteucci, M. D., and Caruthers, M. H., J Am Chem Soc (1981) 103:3185 and can be prepared using commercially available automated oligonucleotide synthesizers. Kinase treatment of single strands prior to annealing or for labeling is achieved using well-known methods.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. If desired, size separation of the cleaved fragments may be performed by polyacrylamide gel or agarose gel electrophoresis using standard techniques. A general description of size separations is found in Meth Enzymol (1980) 65:499-560.

Any of a number of methods are used to introduce mutations into the coding sequence to generate variants of the invention if these are to be produced recombinantly. These mutations include simple deletions or insertions, systematic deletions, insertions or substitutions of clusters of bases or substitutions of single bases. Modifications of the DNA sequence are created by site-directed mutagenesis, a well-known technique for which protocols and reagents are commercially available (Zoller, M J et al., Nucleic Acids Res (1982) 10:6487-6500 and Adelman, J P et al., DNA (1983) 2:183-193)). The isolated DNA is analyzed by restriction and/or sequenced by the dideoxy nucleotide method of Sanger (Proc Natl Acad Sci USA (1977) 74:5463) as further described by Messing, et al., Nucleic Acids Res (1981) 9:309, or by the method of Maxam et al., Meth. Enzymol., supra.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts. In fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521 (1985)) or in the RSV-LTR (from Rous sarcoma virus) (Gorman, C M, Proc. Natl. Acad. Sci. USA 79:6777 (1982). Also useful are the promoter of the mouse metallothionein I gene (Hamer, D., et al., J. Mol. Appl. Gen. 1:273-288 (1982)); the T K promoter of Herpes virus (McKnight, S., Cell 31:355-365 (1982)); the SV40 early promoter (Benoist, C., et al., Nature 290:304-310 (1981)); and the yeast gal4 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)). Other illustrative descriptions of transcriptional factor association with promoter regions and the separate activation and DNA binding of transcription factors include: Keegan et al., Nature (1986) 231:699; Fields et al., Nature (1989) 340:245; Jones, Cell (1990) 61:9; Lewin, Cell (1990) 61:1161; Ptashne et al., Nature (1990) 346:329; Adams et al., Cell (1993) 72:306. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B M, Genes IV, Oxford University Press, Oxford, (1990), pp. 552-576. Particularly useful are retroviral enhancers (e.g., viral LTR). The enhancer is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

The nucleic acid sequences of the invention can also be chemically synthesized using standard techniques. Various methods of chemically synthesizing polydeoxynucleotides are known, including solid-phase synthesis which, like peptide synthesis, has been fully automated with commercially available DNA synthesizers (See, e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

Delivery of Nucleic Acid to Cells and Animals

DNA delivery involves introduction of a “foreign” DNA either (1) into a cell ex vivo and ultimately, into a live animal by administering the cells, or (2) directly into the animal. Several general strategies for “gene delivery” (i.e., delivery of any nucleic acid vector) for purposes that include “gene therapy” have been studied and reviewed extensively (Yang, N-S., Crit. Rev. Biotechnol. 12:335-356 (1992); Anderson, W. F., Science 256:808-813 (1992); Miller, A S, Nature 357:455-460 (1992); Crystal, R G, Amer. J. Med. 92 (suppl 6A):44S-52S (1992); Zwiebel, J A et al., Ann. N.Y. Acad. Sci. 618:394-404 (1991); McLachlin, J R et al., Prog. Nucl. Acid Res; Molec. Biol. 38:91-135 (1990); Kohn, D B et al., Cancer Invest. 7:179-192 (1989), which references are herein incorporated by reference in their entirety).

One approach comprises nucleic acid transfer into primary cells in culture followed by autologous transplantation of the ex vivo transformed cells into the host, either systemically or into a particular organ or tissue.

Preferred DNA molecules for delivery as described below encode Tpm, e.g., SEQ ID NO:1, 3, 5, 7, 9, 11, 13, 15, 17 or 19, or a AALBP fragment thereof (SEQ ID NO:2, 4, 6, 8, 10, 12, 14, 16, 18 or 20) or shorter peptides based on these sequences.

For accomplishing the objectives of the present invention, nucleic acid therapy would be accomplished by direct transfer of a the functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Examples of successful “gene transfer” reported in the art include: (a) direct injection of plasmid DNA into mouse muscle tissues, which led to expression of marker genes for an indefinite period of time (Wolff, J. A. et al., Science 247:1465 (1990); Acsadi, G. et al., The New Biologist 3:71 (1991)); (b) retroviral vectors are effective for in vivo and in situ infection of blood vessel tissues; (c) portal vein injection and direct injection of retrovirus preparations into liver effected gene transfer and expression in vivo (Horzaglou, M. et al., J. Biol. Chem. 265:17285 (1990); Koleko, M. et al., Human Gene Therapy 2:27 (1991); Ferry, N. et al., Proc. Natl. Acad. Sci. USA 88:8387 (1991)); (d) intratracheal infusion of recombinant adenovirus into lung tissues was effective for in vivo transfer and prolonged expression of foreign genes in lung respiratory epithelium (Rosenfeld, M. A. et al., Science 252:431 (1991); (e) Herpes simplex virus vectors achieved in vivo gene transfer into brain tissue (Ahmad, F. et al., eds, Miami Short Reports—Advances in Gene Technology: The Molecular Biology of Human Genetic Disease, Vol 1, Boehringer Manneheim1 Biochemicals, USA, 1991). Gene therapy of cystic fibrosis using transfection by plasmids using any of a number of methods and by retroviral vectors has been described by Collins et al., U.S. Pat. No. 5,240,846.

Retroviral-mediated human therapy utilizes amphotrophic, replication-deficient retrovirus systems (Temin, H. M., Human Gene Therapy 1:111 (1990); Temin et al., U.S. Pat. No. 4,980,289; Temin et al., U.S. Pat. No. 4,650,764; Temin et al., U.S. Pat. No. 5,124,263; Wills, J. W. U.S. Pat. No. 5,175,099; Miller, A. D., U.S. Pat. No. 4,861,719). Such vectors have been used to introduce functional DNA into human cells or tissues, for example, the adenosine deaminase gene into lymphocytes, the NPT-II gene and the gene for tumor necrosis factor into tumor infiltrating lymphocytes. Retrovirus-mediated gene delivery generally requires target cell proliferation for gene transfer (Miller, D. G. et al., Mol. Cell. Biol. 10:4239 (1990). This condition is met by certain of the preferred target cells into which the present DNA molecules are to be introduced, i.e., actively growing tumor cells. The DNA molecules encoding the Tpm polypeptide, domain or peptide fragments of the present invention may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R F et al., Cell 33:153-159 (1983); Miller, A D et al., Molec. Cell. Biol. 5:431-437 (1985); Sorge, J, et al., Molec. Cell. Biol. 4:1730-1737 (1984); Hock, R A et al., Nature 320:257 (1986); Miller, A D et al., Molec. Cell. Biol. 6:2895-2902 (1986). Newer packaging cell lines which are efficient an safe for gene transfer have also been described (Bank et al., U.S. Pat. No. 5,278,056.

This approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M. S., In: Virology, Fields, B N et al., eds, Raven Press, New York, 1990, p. 1679; Berkner, K L, Biotechniques 6:616 9191988), Strauss, S E, In: The Adenoviruses, Ginsberg, H S, ed., Plenum Press, New York, 1984, chapter 11), herpes simplex virus (HSV) for neuron-specific delivery and persistence. Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941 (1991) in the present invention.

Another useful vector, particularly in humans, is vaccinia virus, which can be rendered non-replicating (U.S. Pat. Nos. 5,225,336; 5,204,243; 5,155,020; 4,769,330; Sutter, G et al., Proc. Natl. Acad. Sci. USA (1992) 89:10847-10851; Fuerst, T R et al., Proc. Natl. Acad. Sci. USA (1989) 86:2549-2553; Falkner F G et al.; Nucl. Acids Res (1987) 15:7192; Chakrabarti, S et al., Molec. Cell. Biol. (1985) 5:3403-3409). Descriptions of recombinant vaccinia viruses and other viruses containing heterologous DNA and their uses in immunization and DNA therapy are reviewed in: Moss, B, Curr. Opin. Genet. Dev. (1993) 3:86-90; Moss, B, Biotechnology (1992) 20:345-362; Moss, B, Curr Top Microbiol Immunol (1992) 158:25-38; Moss, B, Science (1991) 252:1662-1667; Piccini, A et al., Adv. Virus Res. (1988) 34:43-64; Moss, B et al., Gene Amplif Anal (1983) 3:201-213.

In addition to naked DNA or RNA, or viral vectors, engineered bacteria may be used as vectors. A number of bacterial strains including Salmonella, BCG and Listeria monocytogenes(LM) (Hoiseth & Stocker, Nature 291, 238-239 (1981); Poirier, T P et al. J. Exp. Med. 168, 25-32 (1988); (Sadoff, J. C., et al., Science 240, 336-338 (1988); Stover, C. K., et al., Nature 351, 456-460 (1991); Aldovini, A. et al., Nature 351, 479-482 (1991); Schafer, R., et al., J. Immunol. 149, 53-59 (1992); Ikonomidis, G. et al., J. Exp. Med. 180, 2209-2218 (1994)). These organisms permit enteric routes of infection, providing the possibility of oral nucleic acid delivery.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N.-S., et al., Proc. Natl. Acad. Sci. USA 87:9568 (1990); Williams, R S et al., Proc. Natl. Acad. Sci. USA 88:2726 (1991); Zelenin, A V et al., FEBS Lett. 280:94 (1991); Zelenin, A V et al., FEBS Lett. 244:65 (1989); Johnston, S A et al., In Vitro Cell. Dev. Biol. 27:11 (1991)). Furthermore, electroporation, a well-known means to transfer genes into cell in vitro, can be used to transfer DNA molecules of the present invention to tissues in vivo (Titomirov, A V et al., Biochim. Biophys. Acta 1088:131 ((1991)).

“Carrier mediated gene transfer” has also been described (Wu, C. H. et al., J. Biol. Chem. 264:16985 (1989); Wu, G Y et al., J. Biol. Chem. 263:14621 (1988); Soriano, P. et al., Proc. Natl. Acad. Sci. USA 80:7128 (1983); Wang, C-Y, et al., Proc. Natl. Acad. Sci USA 84:7851 (1982); Wilson, J M et al., J. Biol. Chem. 267:963 (1992)). Preferred carriers are targeted liposomes (Nicolau, C et al., Proc. Natl. Acad. Sci. USA 80:1068 (1983); Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA of the present invention for transfer.

Plasmid DNA used for transfection or microinjection may be prepared using methods well-known in the art, for example using the Quiagen procedure (Quiagen), followed by DNA purification using known methods, such as the methods exemplified herein.

Diseases and Disorders to be Treated

Malignant and metastatic diseases and conditions (tumors and cancer) which can be treated in accordance with the present invention include, but are not limited to, solid tumors, e.g., carcinomas, sarcomas, lymphomas and other malignant or nonmalignant tumors such as those listed in the table below (for a review of such disorders, see any textbook of clinical oncology, most recent edition, e.g., Cancer: Principles & Practice of Oncology, 5^{th} Ed. (DeVita, V. et al., eds), Philadelphia: Lippincott-Raven Publishers, 1997)

CANCERS/TUMORS

| | | |
|---|---|---|
| Acoustic neuroma | Ewing's tumor | oligodendroglioma |
| Adenocarcinoma | Fibrosarcoma | osteogenic sarcoma |
| Angiosarcoma | glioma | ovarian cancer |
| Astrocytoma | hemangioblastoma | pancreatic cancer |
| Basal cell carcinoma | hepatoma | papillary |
| | | adenocarcinomas |
| Blle duct carcinoma | Kaposi's sarcoma | pinealoma |
| Bladder carcinoma | leiomyosarcoma | prostate cancer |
| Breast cancer | liposarcoma | renal cell carcinoma |
| Bronchogenic | lung carcinoma | retinoblastoma |
| carcinoma | | |
| Cervical cancer | lymphangiosarcoma | rhabdomyosarcoma |
| Chondrosarcoma | lymphangioendotheliosarcoma | sebaceous |
| | | gland carcinoma |
| Choriocarcinoma | medullary carcinoma | seminoma |
| Colon carcinoma | medulloblastoma | small cell |
| | | lung carcinoma |
| Craniopharyngioma | melanoma | squamous cell |
| | | carcinoma |
| Cystadenocarcinoma | meningioma | sweat gland |
| | | carcinoma |
| Embryonal | mesothelioma | synovioma |
| carcinoma | | |
| Endotheliosarcoma | myxosarcoma | testicular tumor |
| Ependymoma | neuroblastoma | Wilms' tumor |

The present invention is directed to the treatment of ocular disorders that involve pathogenic ocular neovascularization such as that associated with, or a cause of, proliferative diabetic retinopathy, neovascular age-related macular degeneration, neovascular glaucoma, retinopathy of prematurity, sickle cell retinopathy, retinal vein occlusion, retrolental fibroplasia, uveitis, corneal graft neovascularization, as well as other eye inflammatory diseases, ocular tumors and diseases associated with choroidal or iris neovascularization.

Other disorders which can be treated in accordance with the present invention include, but are not limited to, uterine disease such as endometriosis, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Therapeutic or prophylactic utility of the present invention and the determination of therapeutically effective dosages can be determined or demonstrated in vivo in a suitable animal model system prior to testing in humans. Such model systems may be based on the use of rats, mice, chicken, cows, monkeys, rabbits, etc. For in vivo testing, prior to administration to humans, any animal model system known in the art may be used. Some preferred model systems have been set forth above.

Having now generally described the invention, the same will be more readily understood through reference to the following examples which are provided by way of illustration, and are not intended to be limiting of the present invention, unless specified.

### EXAMPLES

### Materials and Methods for Examples

Two-chain high molecular weight kininogen (HKa) was purchased from Enzyme Research Laboratories (Bloomington, Ind.). Recombinant bFGF and VEGF were from Becton-Dickinson Biosciences (Franklin Lakes, N.J.). NHS (sulfo)-LC-biotin and Bis(sulfocuccinimidyl) suberate (BS³) were from Pierce (Rockford, Ill.).

The anti-Tpm mAbTM-311, raised against chicken gizzard Tpm, was obtained as ascites from Sigma (St. Louis, Mo.), and purified with Protein A-Sepharose®. Affinity-purified rabbit antibodies that block the binding of HKa to domains 2+3 of the urokinase receptor (uPAR) have been described (Colman, R W et al. (1997) J. Clin. Invest. 100, 1481-1487. A rabbit antibody that blocks HK binding to cytokeratin 1 was from Dr. Alvin Schmaier (Hasan, A A et al. (1998) Proc. Natl. Acad. Sci., U.S.A. 95:3615-3620), and a mAb that blocks binding of HK to the EC gC1qR was from Dr. Berhane Geebreheweit (Joseph, K et al. (1996) Proc. Natl. Acad. Sci. USA 93:8552-8557).

Cloning expression, refolding and purification of kininogen domain 5. Recombinant HKa domain 5 (HKa D5) was produced as a calmodulin binding protein (CBP) conjugate in E. coli. Briefly, domain 5 cDNA was PCR amplified from a full-length HK cDNA using primers

| | |
|---|---|
| 5′-CGGGATCCGTAAGTCCACCCCACACTTC-3′ | (SEQ ID NO:27) |
| and | |
| 5′-CGAATTCTCAGCTTGCCAAATGCTC-3′. | (SEQ ID NO:28) |

The purified PCR product was digested with BamHI and EcoRI and ligated into the expression vector pCAL-n (Stratagene, La Jolla, Calif.). The vector was transformed into BL21(DE3) cells and subclones were grown and induced with 1 mM IPTG. SDS-PAGE revealed that the majority of expressed CBP-HKa D5 was in inclusion bodies. To purify these, the pellet from a 500 ml bacterial culture was lysed, homogenized in 4% Tergitol and centrifuged at 10,000×g for 45 minutes. The purified inclusion bodies were sonicated in 7 M guanidine HCl, and the denatured protein clarified by centrifugation and then added to 1000 ml of 50 mM bicine, pH 8.8, containing 150 mM NaCl. The refolded CBP-HKa D5 was purified by affinity chromatography on HiTrap SP (Amersham-Pharmacia, Piscataway N.J.), then digested with α-thrombin (2.5 μg/mg of CBP-D5). Free HKa D5 was purified using Mono S.

Cell culture. HUVEC were isolated and cultured as previously described (Zhang, J.-C. et al. (2000) FASEB J 14:2589-2600). MDA-MB-231 breast carcinoma cells were obtained from the American Type Culture Collection.

EC proliferation assays. The effect of HKa on ECs in the absence or presence of mAb TM-311 was initially assessed using a proliferation assay, as described in Zhang et al., supra. Relative numbers of cells remaining in each well of a 96 well microplate after incubation for 48 hours in the absence or presence of HKa were determined using the Aqueous® cell proliferation assay (Promega, Madison, Wis.). Results are presented as the percent inhibition of bFGF-induced EC proliferation, which essentially reflects the extent of HKa-induced EC apoptosis. Though bFGF was used as the EC mitogen in most studies of the studies described herein, identical results were obtained using VEGF.

Assessment of EC apoptosis. The effect of TM-311 on HKa- or HKa D5-induced EC apoptosis was determined using several methods. First, staining of control or HKa exposed ECs using 4′, 6′-diamidino-2-phenylindole dihydrochloride (DAPI) (Molecular Probes, Eugene Oreg.) was employed to highlight apoptosis-associated changes in nuclear morphology. Second, apoptosis was assessed by TUNEL staining, using the Apo-Direct kit (Pharmingen, San Diego, Calif.). Cells were counterstained with propidium iodide to define all nuclei, and the percentage of apoptotic CUNEL positive) nuclei determined by manual counting. Finally, to assess endothelial apoptosis by endonucleolytic cleavage of DNA, EC DNA was isolated and separated using 0.8% agarose gel electrophoresis. Gels were stained with ethidium bromide, and DNA visualized using UV light.

Exposure of Tpm on the cell surface. Two approaches were used to detect exposure of Tpm on ECs. First, confluent or proliferating ECs cultured in Lab-Tek® chambers (Nunc, Naperville Ill.) were fixed by exposure to 3.7% paraformaldehyde, blocked using 10% donkey serum and then incubated with either mAb TM-311 or non-immune murine IgG₁. Bound antibody was detected using rhodamine-conjugated donkey anti-mouse IgG, and stained cells were examined using a Bio-Rad MRC 600 laser scanning confocal microscope. For confocal imaging, control stains were set to a black background and positive samples viewed at the same laser intensity, aperture, gain and black level settings. One-micron optical slices were taken for each sample, beginning at the coverslip surface and ending at the apical surface. Projections were acquired using Confocal Assistant Imaging Software, v 4.02. In some experiments, cells were exposed to 0.1% Triton-X-100 in PBS for 15 minutes prior to staining.

To further assess the availability of Tpm on the surface of confluent or proliferating ECs, intact, unfixed cells were labeled using NHS (sulfo)-LC-biotin, as described by (Ma, K et al., (2000) J. Biol. Chem. 275:15541-15548). After labeling, cell extracts were prepared in a buffer containing 0.1 M Tris-HCl, pH 7.4, 1% Triton X-100 and protease inhibitors, and equal amounts of protein from confluent and subconfluent cultures were immunoprecipitated using mAb TM-311. Precipitated proteins were separated by 10% SDS-PAGE, and transferred to a polyvinylidene fluoride (PVDF) membrane. Biotinylated proteins were detected by incubating the membrane with streptavidin-peroxidase and chemiluminescence reagent (Super Signal, Pierce) before exposure to Kodak XL-blue autoradiographic film.

Cross-linking of HKa to ECs. To determine whether HKa interacted with a specific protein on proliferating ECs, the present inventors determined whether it could be cross-linked to such a protein. Biotin-labeled HKa (Ma et al., supra) was incubated with proliferating or confluent ECs for 30 minutes, at 37° C. Cells were then washed and exposed to the bifunctional, membrane-impermeable cross-linker, BS³, for 15 minutes at room temperature. Detergent extracts were prepared, and 40 μg of cell protein from proliferating and confluent cultures separated by 7.5% SDS-PAGE. Proteins were transferred to PVDF, and biotinylated proteins detected using streptavidin-peroxidase and chemiluminescence. To assess the specificity of the cross-linking procedure, studies were also performed in the presence of a 50-fold molar excess of unlabeled HKa, and with MDA-MB-231 breast carcinoma cells.

Binding of HKa to ECs and purified Tpm. Binding of HKa to ECs was measured as described by Hasan, A A et al. (1995) J. Biol. Chem. 270:19256-19261. Briefly, 3×10⁴ HLVEC/ml were cultured in 96-well microplates, then washed and incubated with increasing concentrations of biotin-HKa for two hours, at 4° C. After brief washing, cells were incubated sequentially with a 1:750 dilution of streptavidin peroxidase and the peroxidase substrate, turbo-TMB (Pierce), prior to measurement of A₄₉₀. Absolute amounts of bound HKa were determined by comparison of A₄₉₀ values with a standard curve prepared using known amounts of biotin-HKa Binding was measured in the presence (to determine total binding) and absence (to determine non-specific binding) of 10 μM Zn²⁺, and specific binding defined as the difference between total and non-specific binding (van Iwaarden, F et al. (1988) J. Biol. Chem. 263:4698-4703). The dissociation constant (K_{d}) was determined by fitting the saturation isotherm by nonlinear regression using the Prism software program (GraphPad, San Diego, Calif.).

In selected experiments, the ability of mAb TM-311 to inhibit the binding of HKa to cells was assessed by incubating biotin-HKa with ECs in the presence of increasing concentrations of antibody. The concentration of TM-311 that inhibited HKa binding by 50% (IC₅₀) was determined from plots of bound HKa versus the log of the TM-311 concentration.

To measure the binding of HKa to purified Tpm, 96 well microplates were coated with 20 μg/ml of chicken gizzard Tpm (Sigma), or bovine serum albumin (BSA), as a control. Wells were then blocked by incubation with phosphate buffered saline containing 5% nonfat milk, and incubated with increasing concentrations of HKa (0.01-20 nM) for 2 hours. Specifically-bound HKa was quantitated as described for cell binding assays (van Iwaarden et al., supra, and by assessing the ability of a 100-fold molar excess of unlabeled HKa to compete with biotin-HKa for binding.

In selected experiments, the ability of mAb TM-311 and recombinant HKa D5 to inhibit the binding of biotin-HKa to Tpm was assessed. These studies allowed determination of the K_{d} for binding of HKa D5 to Tpm using the equation:
K_{d (D5)}=IC₅₀/(1+[HKa]/K_{d (HKa)})
where IC₅₀ is the concentration of HKa D5 that inhibited HKa binding by 50%, and K_{d(HKa)} is the K_{d} for binding of HKa to Tpm.

Chick chorioallantoic membrane assay. The chick chorioallantoic membrane (CAM) assay was used to assess the role of Tpm in the antiangiogenic activity of HKa in vivo (Nguyen, M et al. (1993) Microvasc Res 47:40). Three day-old fertilized White Leghorn chicken eggs were cracked in sterile Petri dishes. Embryos were cultured at 37° C., under 4% CO₂, until day 7, at which time a 3.0 mm filter disc containing either 30 ng bFGF (positive control), 30 ng bFGF and 10 μg HKa or 30 ng bFGF, 10 μg HKa and 20 μg of mAb TM-311 was placed on the CAM. Each experimental condition was tested in at least 6 eggs. On day 10, embryos were photographed using a SPOT digital camera Angiogenesis was quantitated by counting the number of neovessels in direct contact with the filter disc.

### Example I

### Antibody Specific for Tpm Blocks the Action of HKa on ECs

Preliminary studies aimed at defining the structure of Zn²⁺-bound HKa D5 (Kumar, G A et al., 200, Abst. Am. Chem. Soc. 222:134) suggested structural homology between HKa D5 and endostatin, a Zn²⁺-binding, antiangiogenic polypeptide comprised of the NC domain of collagen XVIII (O'Reilly, M S et al (1997) Cell 88:277-285; Ding, Y-H et al. (1998) Proc. Natl. Acad. Sci., U.S.A. 95:10443-10446).

Prior studies by one of the present inventors and colleagues could not demonstrate involvement of previously-reported EC binding sites for HK or HKa in the antiangiogenic effects of this polypeptide (Zhang et al., supra). A recent report suggested that the anti-angiogenic activity of endostatin was mediated through binding to Tpm (MacDonald, N J et al. (2001) J. Biol. Chem. 276:25190-25196). The present inventors therefore sought to determine if Tpm functioned in a similar way with HKa.

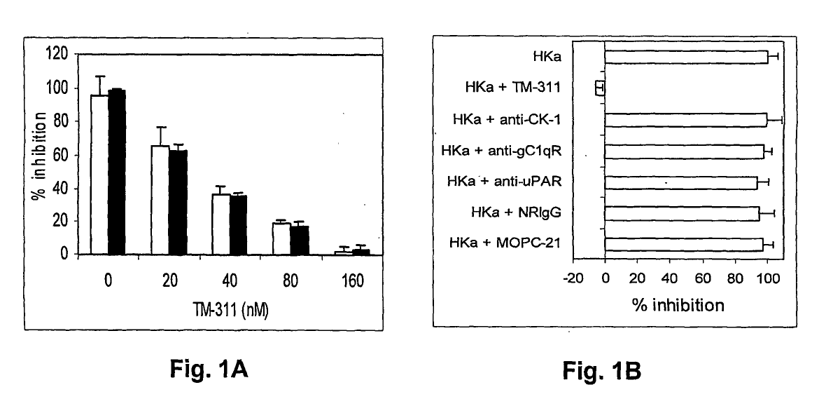

The initial study was designed to determine whether mAb TM-311 affected the ability of HKa to block growth factor-induced EC proliferation. TM-311 indeed blocked, in a concentration-dependent manner, the HKa and D5-mediated inhibition of bFGF- and VEGF-induced EC proliferation (FIG. 1A). Antibodies that block the binding of HKa to different receptors, the urokinase receptor (Colman, R W et al. (1997) J. Clin. Invest. 100:1481-1487), gC1qR (Joseph et al, supra; Herwald, H et al. (1996) J. Biol. Chem. 271:13040-13047) and cytokeratin 1 (Hasan et al., supra) did not have such an effect (FIG. 1B).

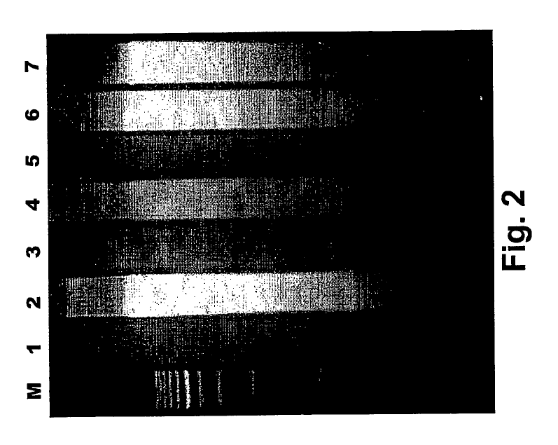

These effects reflected inhibition of HKa-induced EC apoptosis by TM-311. TM-311 prevented the endonucleolytic fragmentation of DNA (FIG. 2) as well as the characteristic apoptotic changes in nuclear morphology and the increase in TUNEL-positive cells following exposure of proliferating ECs to HKa or HKa D5 (Zhang et al., supra). Non-immune murine IgG (MOPC-21 myeloma protein) also lacked any such activity.

The specificity of these effects was addressed by evaluating the ability of mAb TM-311 to inhibit EC induced by 2-methoxyestradiol, another anti-angiogenic agent with such activity (Yue, T L et al. (1997) Mol Pharm 51:951-962). However, even at a very high concentration of 6 μM, mAb TM-311 did not inhibit EC apoptosis induced by 2 μM 2-methoxyestradiol (FIG. 2, lanes 6 versus 7).

### Example II

### Tpm is Present on the Surface of Activated EC's

The results of Example I suggested an essential role for Tpm in mediating HKa-induced EC apoptosis. However, Tpm is a cytoskeletal protein, and there have been no reports of it being exposed on the endothelial surface. Indeed, in only one prior study was a single isoform of Tpm, hTM5, observed on the surface of any cell type-colon epithelial cells and cells of a colon carcinoma line (Kesari K V et al., Clin. Exp. Immunol. (1999). 118:219-27). Although a recent report suggested that the antiangiogenic activity of endostatin requires interaction with Tpm, it was hypothesized that internalization of endostatin was necessary for this to occur (McDonald et al., supra).

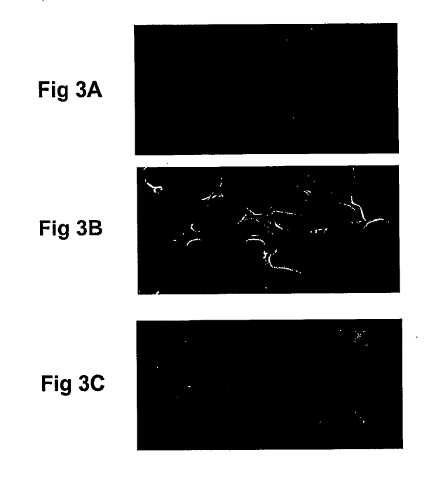

To assess whether Tpm is expressed on the EC surface, and to address the selectivity of HKa for proliferating ECs, the present inventors used confocal scanning laser microscopy to assess proliferating and confluent cultures of ECs stained with mAb TM-311. Proliferating cells stained specifically with TM-311 (FIGS. 3A versus 3B), and the surface staining of these cells was more prominent than with confluent cells (FIGS. 3B versus 3C). The surface staining pattern of proliferating cells was unchanged when cells were permeabilized by exposure to 0.2% Triton X-100 prior to staining, though an intracellular pool of Tpm, particularly evident in the confluent cells (FIG. 3C), was more prominent.

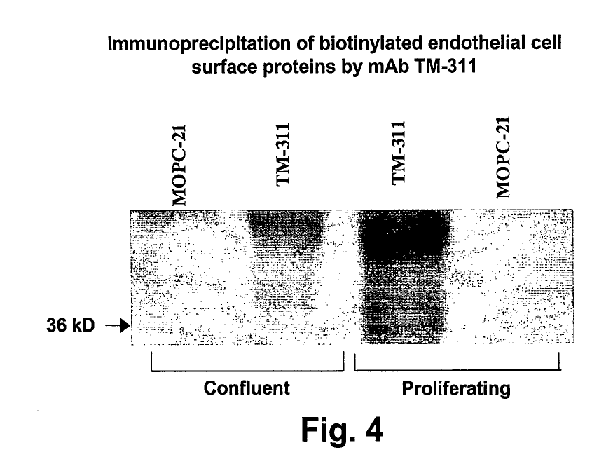

To confirm these results of the existence of Tpm on the EC surface, surface proteins on proliferating or confluent ECs were labeled with biotin, and detergent extracts of the labeled cells were immunoprecipitated with mAb TM-311. TM-311 precipitated proteins of ˜36 kDa and ˜40 kDa, consistent with the expected molecular weight of Tpm (Lees-Miller, J P et al. (1991) Bioessays 13:429-437; Lin, J J C et al., (1997) Int. Rev. Cytology 170:1-38), from both EC cultures (FIG. 4). However, these proteins were precipitated in markedly greater amounts from the proliferating cells (FIG. 4).

Characterization of the Binding of HKa to ECs and Tpm

Several approaches were used for further evaluation of the possibility that HKa binds to Tpm on ECs.

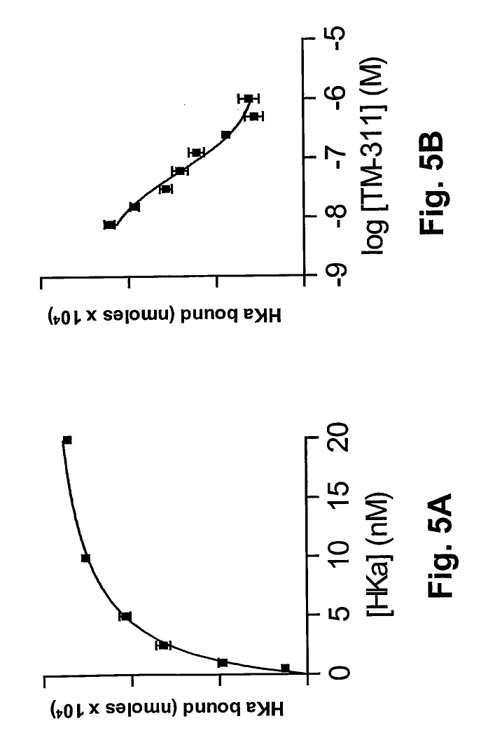

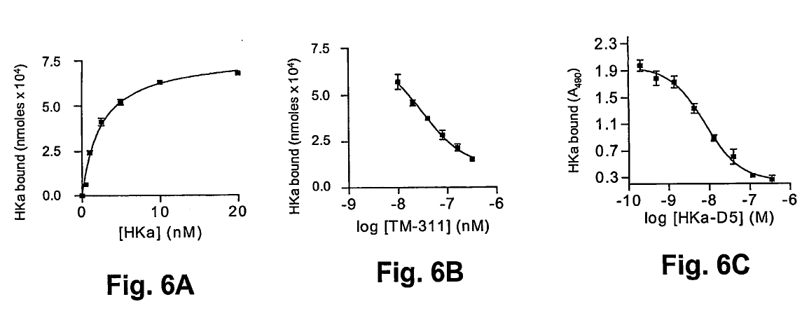

First, the ability of mAb TM-311 to block the binding of HKa to subconfluent ECs was tested. Biotin-HKa bound specifically to these cells in a Zn²⁺-dependent manner, with a K_{d} of ˜2.5 nM (FIG. 5A). TM-311, but not a control IgG (MOPC-21), blocked the specific binding of HKa to cells by about 90% (FIG. 5B) Next, the binding of HKa to purified Tpm immobilized in 96-well microplates was studied. The results are shown in FIGS. 6A-6C/PNAS. HKa bound with similar affinity (K_{d} of about 2.6 nM), and in a Zn²⁺-dependent manner, to purified Tpm (FIG. 6A). mAb TM-311 (FIG. 6B), as well as HKa D5 (FIG. 6C) blocked binding, confirming that HKa D5 binds with high affinity to Tpm (estimated K_{d} about 2.1 nM).

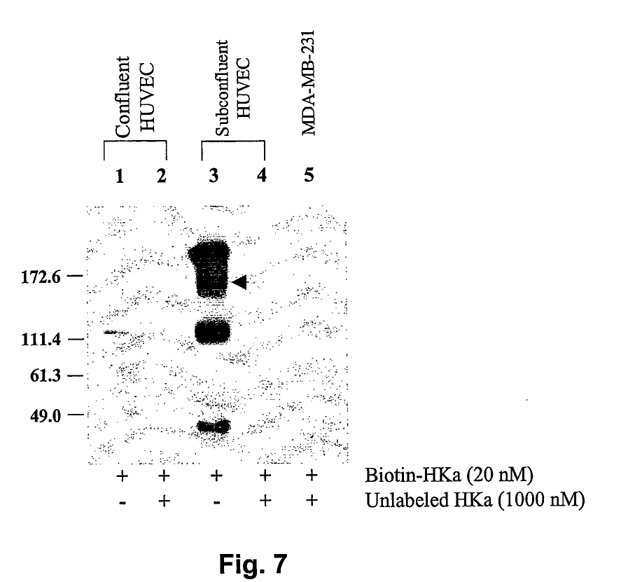

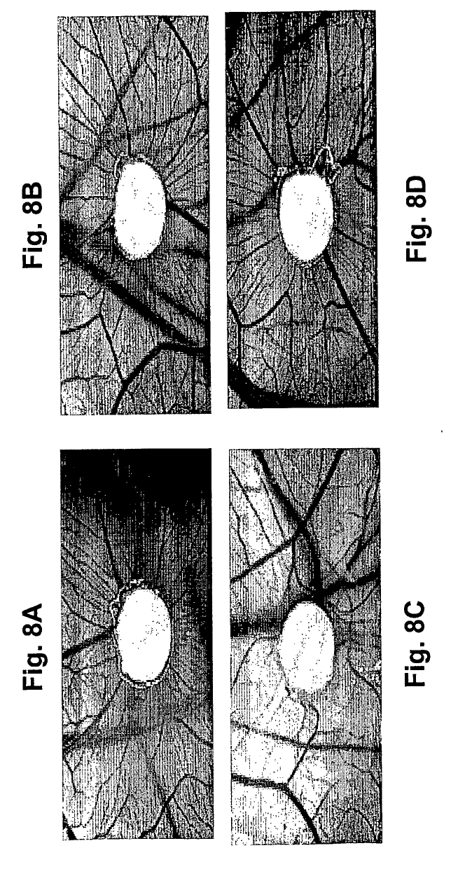

New FIG. 7 shows direct binding of mAb TM-311 to immobilized Tpm by ELISA. FIG. 8 provides additional proof that this antibody competes with HKa for binding to such immobilized Tpm.

Finally, a study was done to determine whether biotinylated HKa could be cross-linked to an EC surface protein of the size of Tpm. When incubated with confluent ECs prior to exposure to the membrane-impermeable cross-linker, BS³, biotin-HKa was barely detectable in cell extracts (FIG. 5, lane 1). However, HKa was detected in both an uncomplexed form (Mᵣ ˜110 kDa) and within a broad, high molecular weight complex (FIG. 5, lane 3) in extracts of proliferating cells. A complex between HKa and Tpm would be expected to exhibit a Mᵣ of ˜140-150 KDa. Indeed, a discrete band of this size was observed within the broad band (FIG. 5, arrow).

The specificity of this interaction is supported by the observations that complex formation was prevented by excess unlabeled HKa (FIG. 5, lane 4), and that high molecular weight complexes were not observed when the cells were MDA-MB-231 breast carcinoma cells rather than ECs (FIG. 5, lane 5).

The presence of HKa within complexes having a molecular size >150 kDa may result from association of the HKa-Tpm complex with actin or other Tpm binding proteins, or complexes between HKa and other EC binding proteins.

### Example III

### MAb Tm 311 Blocks the Anti-Angiogenic Effects of HKa In Vivo

The CAM assay was employed to assess the functional role of Tpm in an in vivo angiogenesis model. The results are shown in FIG. 8A-8D. HKa inhibited angiogenesis induced by bFGF-containing filter discs by about 85%, as determined by vessel counts (FIG. 8A vs. FIG. 8C). Similar results were obtained using HKa D5 (Colman, R W et al. (2000) Blood 95:543-550). However, the anti-angiogenic effect of HKa was blocked by TM-311 (FIG. 8C vs FIG. 8D), whereas non-immune murine IgG was without effect. These results demonstrate that Tpm mediates the anti-angiogenic activity of HKa in vivo as well as in vitro, ard hence may function as an “anti-angiogenic” binding site for HKa.

Discussion of Examples I-III

The result presented herein prove that that HKa binds with high affinity to Tpm through interactions involving the HKa D5 domain, and that inhibition of this interaction by an anti-Tpm antibody blocks (a) the induction of EC apoptosis and (b) the inhibition of angiogenesis by HKa. These observations provide compelling evidence that the effects of HKa on proliferating ECs are mediated through direct binding to Tpm.

In addition to identifying a new EC binding site for HKa, the present invention provides insight into the biology of ECs during angiogenesis by demonstrating that Tpm, a cytoskeletal protein, is preferentially exposed on the surface of proliferating cells. Exposure of Tpm on the endothelial surface was documented by (a) direct visualization and (2) the fact that Tpm could be biotinylated NHS (sulfo) LC-biotin.

Earlier studies examining the binding of HK or HKa to ECs used confluent, static EC monolayers; however, the present findings demonstrate that under these conditions Tpm is minimally available on the cell surface, leaving other binding sites to the more prominent role in binding single chain (Hasan et al., supra; Herwald et al., supra; Hasan, A A et al, (1994) J. Biol. Chem. 269, 31822-31830; Herwald, H et al. (1995) J. Biol. Chem. 270:14634-14642; Dedio, J et al (1996) FEBS Lett. 399:255-258; Dedio, J et al., (1998) J. Immunol. 160:3534-3542) or two-chain (Colman et al., 1997, supra) high molecular weight kininogen.

These findings are consistent with studies demonstrating that the EC cytoskeleton undergoes dramatic structural rearrangement during the transition between a quiescent and proliferative state (Ingber, D E (1997) Annu Rev Physiol 59:575-599; Ingber, D E et al. (1995) J. Biomechanics 28:1471-1484; Huang, S et al. (2000) Exp. Cell Res. 261:91-103).

However, other than one report in which actin was demonstrated on the surface of cultured ECs (Dudani, A K et al (1996) Br. J Haematol 95:168-178), there has been little evidence for exposure of cytoskeletal components on the EC surface. Hence, the present results challenge the paradigm that the cytoskeleton exists solely within the confines of the EC plasma membrane under all conditions. Moreover, the observation that mAb TM-311 inhibits the anti-angiogenic activity of HKa suggests that Tpm is available on the surface of angiogenic ECs in vivo.

Vertebrate cells express a number of Tpm isoforms in a cell-specific manner. The expression of Tpm in nonmuscle cells has been most intensively studied in human fibroblasts, which express at least eight isoforms (Lin et al, supra). The present inventors have obtained results suggesting that ECs also express multiple Tpm isoforms.

The major role of Tpm is the binding and stabilizing actin filaments, protecting them from separation or depolymerization by factors such as gelsolin or actin depolymerizing factor (Lin et al., supra; Ishikawa, R et al. (1989) J. Biol. Chem. 264:7490-7497). Alterations in Tpm isoform expression has been reported to play a role in cellular transformation (in et al., supra; Takenaga, K et al., (1988) Molec. Cell Biol 8:3934-3937; Pradad, G L et al., (1993) Proc. Natl. Acad. Sci., USA 90:7039-7043), suggesting that Tpm may influence the state of cytoskeletal organization. According to the present invention, alterations in the expression and cellular localization of Tpm contributes to the transition of ECs from a quiescent to an “angiogenic” phenotype.

The identification of Tpm as an endothelial binding site required for the anti-angiogenic activity of HKa raises several questions concerning the mechanism(s) by which HKa induces EC apoptosis. Though HK and HKa have anti-adhesive properties (Colman, R W et al. (1997) Blood 90:3819-3843; Chavakis, T et al., (2000) Blood 96:514-522), the ability of HKa to induce apoptosis of proliferating ECs at concentrations below those generally associated with the anti-adhesive activity argues in favor or additional mechanisms to account for HKa's anti-angiogenic activity (Zhang et al., supra). The results disclosed above suggest that HKa may affect the EC cytoskeleton directly, perhaps causing secondary alterations in downstream signaling pathways dependent upon reciprocal interactions between the cytoskeleton and integrin adhesion receptors. Indeed, biomechanical influences mediated through the cytoskeleton play a critical role in vital processes such as cell cycle entry, cellular growth, and apoptosis (Ingber et al., supra; Huang et al., supra).

A recent report by MacDonald et al., supra, disclosed that an antibody reactive with an endostatin-binding cyclic peptide cross-reacted with human Tpm 3 (hTM3) and that hTM3 may be involved in the anti-angiogenic effects of endostatin in vivo. Both this antibody and mAb TM-311, employed in the above studies, recognize a EC protein having a molecular weight of about 38 kDa, suggesting that mAb TM-311, the Tpm isoform specificity which has not been well characterized, also recognizes hTM3.

Since TM-311 blocked HKa-induced EC apoptosis, it is possible that hTM3 may mediate the antiangiogenic activity of HKa as well. However, in the present studies, TM-311 recognized two EC proteins of ˜36 and ˜40 kDa (FIG. 3), so that any specific role for hTM3 in mediating the activity of HKa is not conclusive. If indeed HKa binds hTM3, then the this interaction may differ from that of endostatin, since the affinity of HKa for Tpm (K_{d}˜2.6 nM) is orders of magnitude higher than that of endostatin (K_{d}˜100 μM). Moreover, MacDonald et al. supra, suggested that endostatin may require internalization prior to interacting with hTM3, while the high molecular weight kininogen is not internalized by ECs (Hasan, A A et al., (1995) Blood 85:3134-3143).

Because the present results support the conception that HKa binds directly to cell surface-exposed Tpm, it may be useful to reconsider the requirement that endostatin be internalized prior to its interaction with Tpm. Nevertheless, the observation that at least two antiangiogenic polypeptides bind to ECs through interactions with Tpm favors a broader role for Tpm, and perhaps other cytoskeletal proteins, in mediating the activity of naturally-occurring angiogenesis inhibitors.

In summary, the present invention is based on the discovery of a novel interaction between HKa and EC Tpm which underlies the antiangiogenic activity of HKa. This also serves as a basis for the development of novel agents targeted toward arrest of pathologic angiogenic processes.

### Example IV

### Binding of HKa and Other Antiangiogenic Agents to Tpm

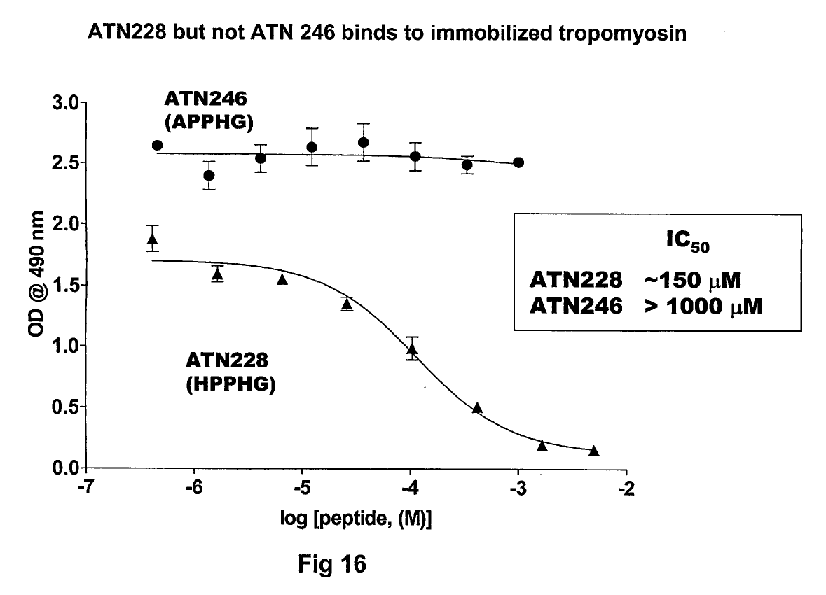

The binding affinity of HKa and its domain D5 (which possesses most, if not all, of the anti-angiogenic activity) to Tpm were measured in a plate binding assay such as that described above in which chicken gizzard Tpm was immobilized to the plate. As shown in FIG. 15, Kd values obtained for both proteins were approximately 1 nM. This value is identical to that obtain in vivo with HUVECs.

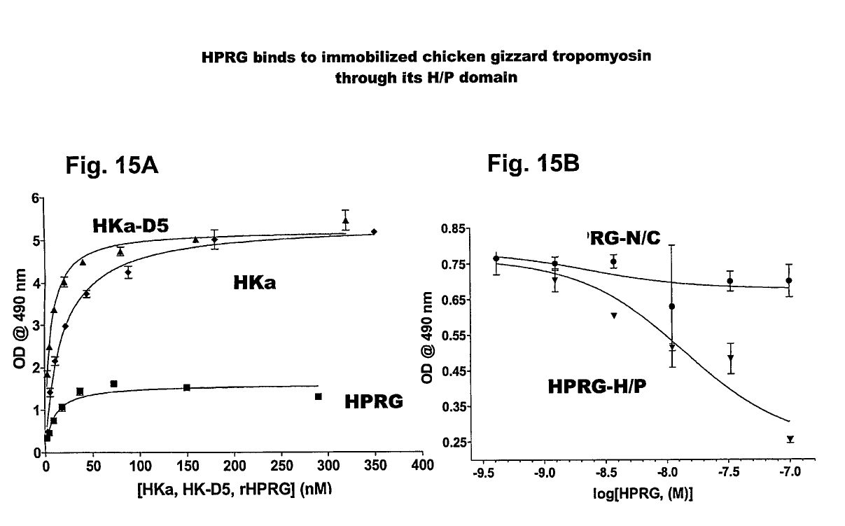

Other human Tpm human isoforms were tested in this same assay with similar results. Several other anti-angiogenic proteins also bind to Tpm. Endostatin binds to immobilized Tpm with a K_{d} of about 2 μM which is 50-fold better than that reported previously (measured by SPR), but is approximately 1,000-fold lower than the affinity of HKa, or HKa-D5 binding (FIGS. 14 and 15).

Troponin I is a component of the Troponin complex that binds and regulates actin contractility in muscle cells, which has also being found to be an anti-angiogenic molecule. Troponin I appears to bind to immobilized Tpm with a Kd in the nanomolar range

### Example V

### Tpm Binding and Inhibition of Angiogenesis by Short Peptides Derived from HPRG

The abundant multi-domain plasma protein Histidine-Prolne-Rich Glycoprotein (HPRG) has been shown by some of the present inventors and their colleagues to have robust anti-angiogenic properties (U.S. Ser. No. 60/268,370 and U.S. Ser. No. 10/074,225, filed Feb. 14, 2002, all of which are incorporated by reference in their entirety).

Interestingly, rabbit HPRG and the Histidine-Proline rich (“H/P”) domain of HPRG, which is responsible for anti-angiogenic activity, bind to Tpm with high affinity and specificity (FIGS. 15A and 15B). The calculated Kd, ˜2 nM, is similar to the Kd of HKa or HKa-D5 binding to Tpm. As a control, the rabbit N/C fragment, which corresponds to all of HPRG minus the H/P domain, shows no binding at the concentration tested (FIGS. 15A and B).

The H/P domain of rabbit HPRG is composed of 2 repeats of HHPHG [SEQ ID NO:29], 7 repeats of PPPHG [SEQ ID NO:30], 6 repeats of HPPHG [SEQ ID NO:31]. The human H/P domain contains 10 tandem repeats of the sequence HHPHG. A combined consensus sequence of human and rabbit is designated [H/P][H/P]PHG [SEQ ID NO:32].

In an effort to identify the minimal sequence within the H/P domain with significant anti-angiogenic activity, the present inventors synthesized and evaluated three peptides from the above mentioned consensus sequence (HHPHG, HPPHG and PPPHG). The binding affinity to immobilized Tpm of those three peptides together with activity in the Matrigel Plug assay was determined and is summarized in Table 5.

The IC₅₀ for the displacement of biotinylated-HKa for the peptides shown in Table 5 was determined as follows: 10 nM biotin-HKa is added to a 96 well plate previously coated with 200 ng of chicken gizzard Tpm in the presence of 10 μM ZnCl₂. Increasing amounts of the peptides are added to the wells. The remaining biotin-HKa bound to Tpm was detected using avidin-HRP and a chromogenic substrate. A Kd was determined by non-linear regression analysis of the empirical data. The effect on the Matrigel Plug assay was determined as follows: Matrigel (0.5 mL) containing 400 ng/ml of bFGF, 50 μg/ml heparin with or without the peptide (300 μM) or saline buffer are injected in the flanks of a mouse. After five days, the plugs are removed and the plugs scanned.

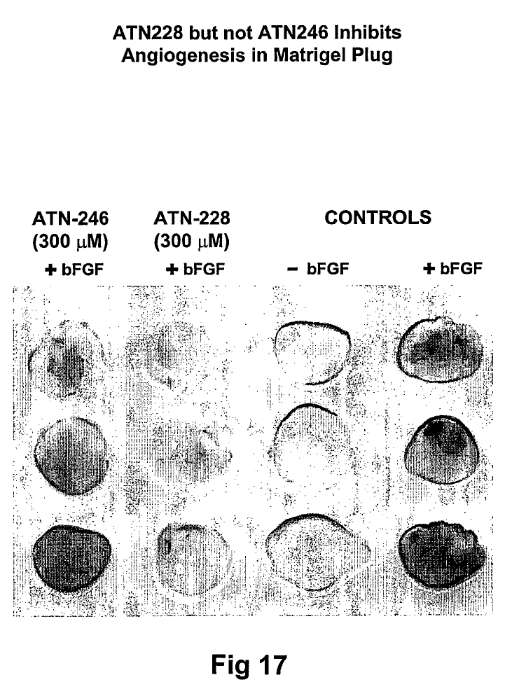

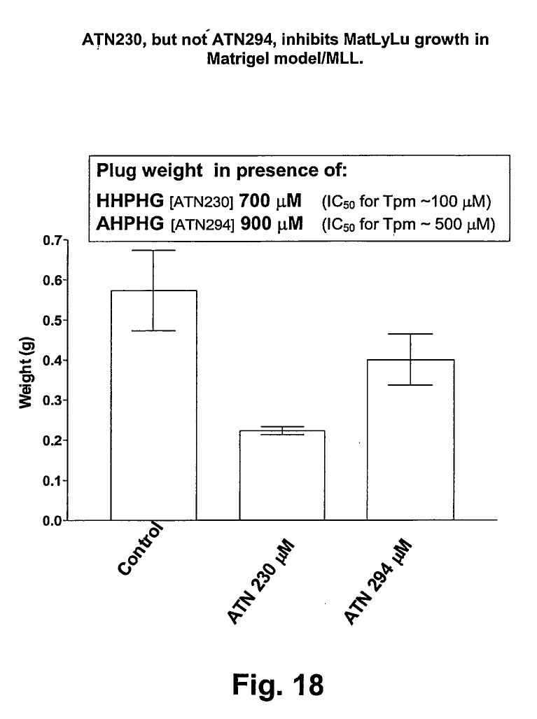

The peptide HHPHG binds Tpm with ˜100 μM affinity and has substantial activity in the Matrigel Plug assay. Alanine substitution of the first His in HPPHG (to yield APPHG) results in complete loss of Tpm binding and anti-angiogenic activities (FIGS. 16 and 17). Thus, loss of Tpm binding activity went hand-in-hand with a loss of anti-angiogenic activity in the Matrigel plug.

TABLE 5

Activity of consensus sequence peptides from
HK-D5 (⋆) or HPRG-H/P domain

| | | | | |
|---|---|---|---|---|
| | | SEQ. | | |
| | | ID | IC₅₀ | Inhibits Angiogenesis |
| ATN# | Sequence | NO: | (μM) | (Matrigel ® assay) |

| | | | | |
|---|---|---|---|---|
| ATN16 ⋆ | HKNKGKKN | 34 | 99.5 | Yes |
| ATN232 ⋆ | TRRHDWGH | 35 | 140.5 | Yes |
| ATN230 | HHPHG | 29 | 121 | Yes |
| ATN239 | c-HHPHG-c | 29 | NB* | |
| ATN269 | c-HHPHG | 29 | NB | |
| ATN228 | HPPHG | 31 | 150 | Yes |
| ATN246 | APPHG | 33 | NB | No |
| ATN231 | c-HPPHG-c | 31 | NB | |
| ATN227 | PPPHG | 30 | NB | |
| ATN229 | c-PPPHG-c | 30 | NB | |

ATN#- Attenuon code number
The IC₅₀ is determined in the Tpm binding assay; *NB = no binding (i.e., IC₅₀ > 10 mM)
C = acetyl or amide cap

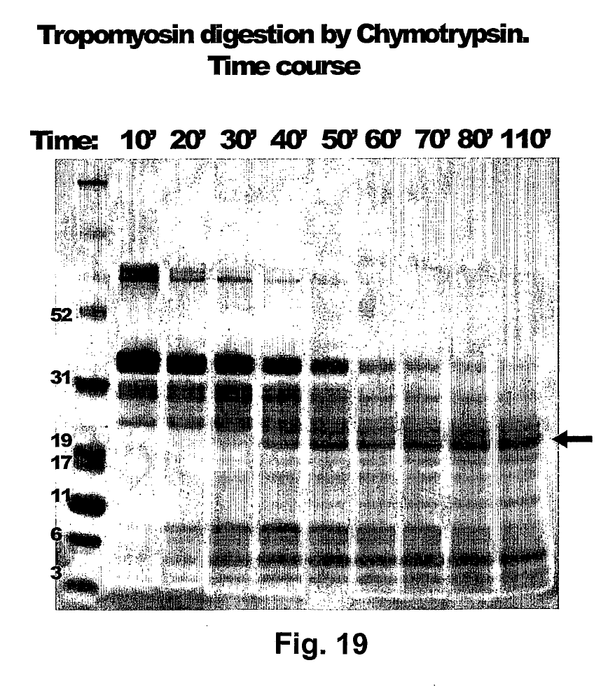

HHPHG also showed anti-tumor activity against MatLyLu prostate cancer cells, whereas the Ala substituted mutant, AHPHG (SEQ ID NO:36), in which binding to Tpm int vitro was abolished, also has significantly less anti-tumor activity (FIG. 18).

The N-terminus of the pentapeptide HHPHG has been derivatized (capped) in an attempt to increase the pentapeptides' affinity to Tpm by increasing productive molecular contacts (or interactions). Table 6 shows a few of these derivatives including the capping groups that have already better affinities for Tpm than the parent compound. The IC₅₀ for the displacement of biotinylated-HKa for the capped peptides shown in the table was determined as follows: 10 nM biotin-HKa was added to a 96 well plate previously coated with 200 ng of chicken gizzard Tpm in the presence of 10 μM ZnCl₂. Increasing amounts of the peptides were added to the wells. The remaining biotin-HKa bound to Tpm was detected using avidin-HRP and a chromogenic substrate. A Kd was determined by non-linear regression analysis of the empirical data

TABLE 6
Activity of N-capped peptides from HPRG-H/P domain

| ATN# | Common Name | IC₅₀(μM) |
|---|---|---|
| ATN-278.000.01 | Fmoc | 55 |
| ATN-276.000.01 | 4-Chlorobenzyl | 23 |
| ATN-281.000.01 | 3-(1-adamantyl)propanoyl | 59 |
| ATN-275.000.01 | (1S)-(+)-camphorsulphonyl | 30 |

### Example VI

### HPRG and the H/P Domain Inhibit Angiogenesis Stimulated by FGF-2 in Matrigel® Plug Model in Vivo

The present inventors identified the region of Tpm that binds to HKa-D5. Chicken gizzard Tpm, because of its high degree of homology to different human Tpm isoforms, has been used as a model.

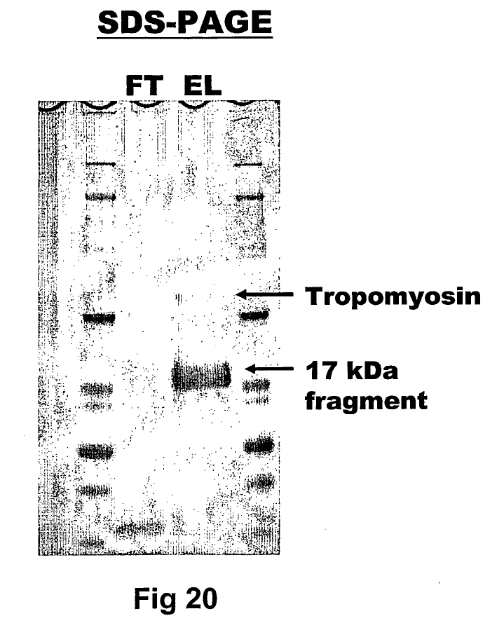

This Tpm was partially proteolyzed with chymotrypsin. A fragment of approximately 20-25 kDa was identified as being enriched during proteolysis by SDS-PAGE (FIG. 19). This fragment bound to the HKa-D5 domain immobilized in the Sepharose resin suggesting that it contains the region that binds to HKa-D5.

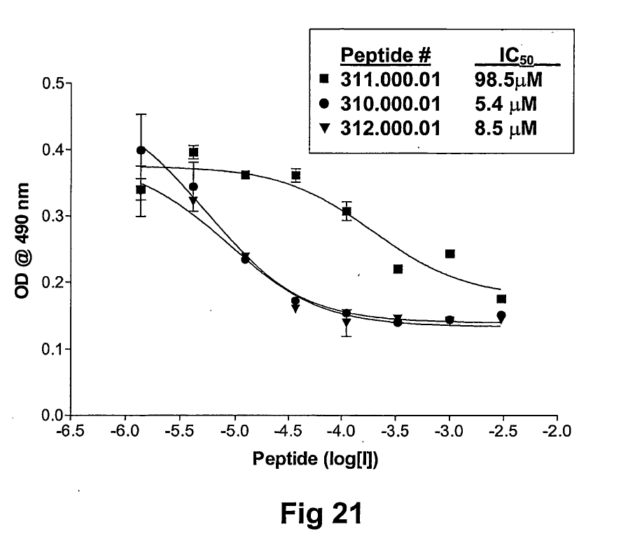

The fragment was partially purified by passing the chymotryptic digest through an affinity column of Sepharose-HKa-D5. The column was extensively washed and eluted with high salt buffer. The resulting material was run on an SDS gel which appears in (FIG. 20). The isolated chymotryptic fragment of Tpm, isolated as above was dialyzed against water and submitted to N-terminal sequencing (University of California, San Diego: Protein Sequencer Facility) and mass spectroscopy (MALDI-TOF) at the Scripps Research Institute Mass Spectrometry facility). The N terminal sequence of this fragment is shown above as residues 61-69 of SEQ ID NO: 1 (or residues 1-9 or SEQ ID NO:2).

The estimated molecular mass of this fragment from MALDI-TOF analysis was ˜17 kDa. The actual molecular weight of the polypeptide based on its sequence is 17,684.6 Da.

### Example VII

### Inhibition of Angiogenesis by Cyclic Peptides that Bind to HKa D5

The following three cyclic peptides were discovered to bind HKa-D5 and were tested here. 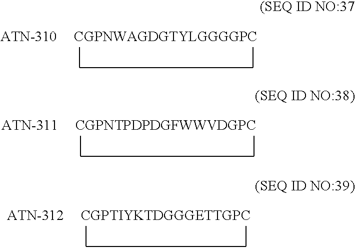
These cyclic peptides were stabilized by a disulfide link between the two terminal Cys residues, indicated above as a “line” or “bond” between the terminal C (cysteine) residues.

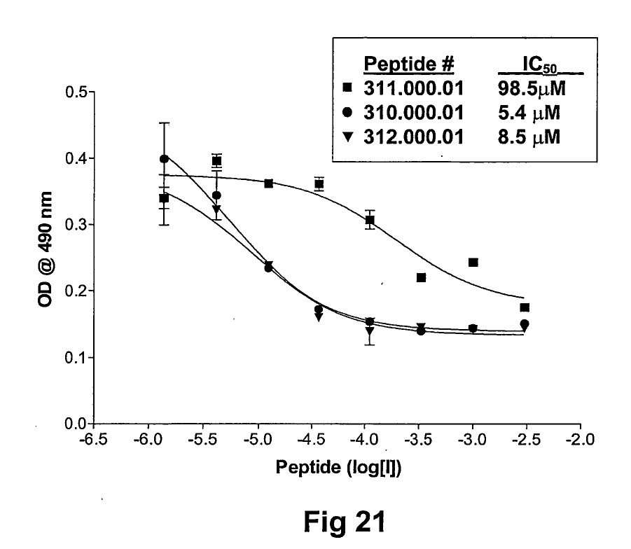

A study was performed to examined whether these peptides displace HKa bound to immobilized Tpm. The results shown in FIG. 21.

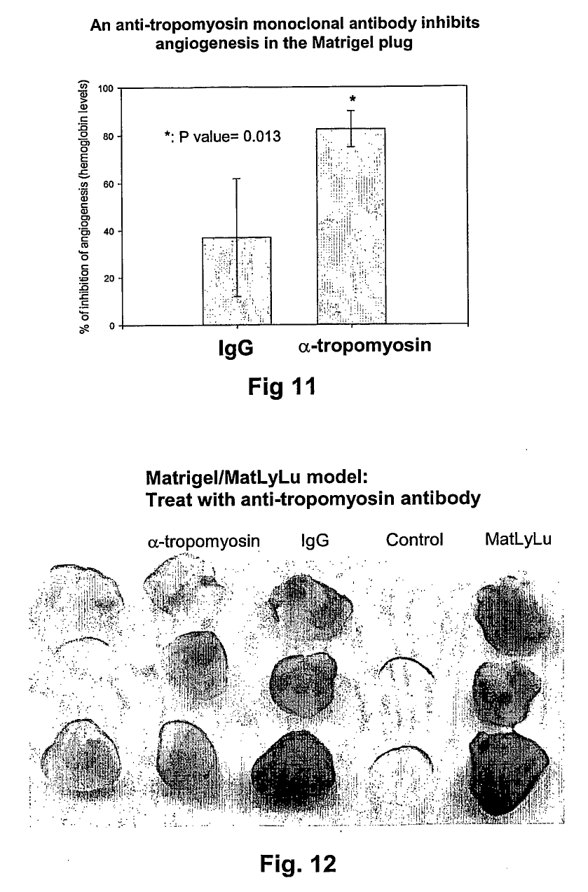

The binding results presented in FIG. 21 show that the cyclic peptides ATN-310, ATN-311 and ATN-312 (referred to in the Figure insert as 310.000.01, 311.000.01 and 312.000.01, respectively) displaced HKa that is bound to Tpm whereas a control ATN-246 did not (not shown). In this experiment, 10 nM biotin-HKa was added to a 96 well plate previously coated with 200 ng chicken gizzard Tpm in the presence of 10 μM ZnCl₂. Increasing amounts of the three cyclic peptides were added to wells as indicated. Bound biotin-HKa was detected and Kd was calculated as in the FIG. 10 description.

These cyclic peptides were also tested for their ability to inhibit bFGF-induced angiogenesis in vivo in a Matrigel plug assay. Matrigel (0.5 mL) containing 400 ng/ml of bFGF, 50 μg/ml heparin with or without 10 μM peptide (ATN-310, ATN-311 or ATN-312 or saline buffer for the controls, was injected subcutaneously in the hind flanks of a mouse as described above. All the treatments were done in triplicate. After five days, the vascularization of the Matrigel plug was determined fluorometrically after i.v. injection of 100 μl of dextran conjugated with fluorescein isothiocyanate (FITC-dextran; MW 250,000, Sigma). Five days after injection, the plugs were removed and subjected to mincing of tissue which releases vascular FITC-dextran into the supernatant. Samples of supernatant were subjected to fluorimetry as a measure of vascularity of the plug. The amount of vasculature (neovessels) is directly proportional to the fluorescent signal which reflects the total vascular blood volume of the plug. The results are shown in Table 7, below and expressed as arbitrary fluorescence units or as % inhibition relative to the negative control.

TABLE 7
Inhibition of Matrigel Plug Angiogenesis In Vivo by ATN-310, ATN-311
and ATN-312

| | Saline | | | |
|---|---|---|---|---|
| | Control | ATN310 | ATN311 | ATN312 |
| Mean¹ | 0.2259 | 0.02760 | 0.0277 | 0.0413 |
| ±SD | 0.0206 | 0.0070 | 0.0110 | 0.0089 |
| % Inhibition | 0 | 87.8 | 87.7 | 81.7 |

¹Arbitrary fluorescence units

It was concluded that at this concentration, all three peptides were very effective inhibitors of angiogenesis. Inhibition of angiogenesis was also observed at lower concentrations of these cyclic peptides.

The references cited above are all incorporated by reference, whether specifically incorporated or not (as are the references cited therein).

Having now fully described this invention, it will be appreciated by those skilled in the art that the same can be performed within a wide range of equivalent parameters, concentrations, and conditions without departing from the spirit and scope of the invention and without undue experimentation.

1. An isolated tropomyosin (Tpm)-related anti-angiogenic receptor polypeptide or peptide which,
(a) is a fragment of a full length native Tpm protein expressed on the surface of endothelial cells or a variant of said fragment,
(b) has a molecular mass of about 17 kDa and corresponds in its sequence to, or is a variant of, an internal fragment of a native Tpm isoform which is a binding site for antiangiogenic polypeptide agents, and
(c) binds to said antiangiogenic polypeptide agents which bind to said native Tpm internal fragment binding site;
wherein
said peptide has between about 4 and about 40 amino acids; and
said variant of the polypeptide or peptide is a conservative substitution variant of a native Tpm sequence; and
said isolated anti-angiogenic receptor polypeptide, peptide or variant has substantially the same biochemical activity of binding to said antiangiogenic polypeptide agents as does said native Tpm internal fragment.

2. The isolated polypeptide, peptide or variant of claim 1 wherein the native Tpm isoform has an amino acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, and SEQ ID NO:19.

3. The isolated polypeptide or peptide or variant of claim 1, wherein the internal fragment of said native Tpm has an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, and SEQ ID NO:20.

4. The isolated polypeptide, peptide or variant of claim 1 wherein the Tpm isoform is a human Tpm isoform.

5. The isolated polypeptide, peptide or variant of claim 1 wherein said antiangiogenic polypeptide agent which binds to said isolated polypeptide or peptide is selected from the group consisting of:
(a) human histidine-proline rich glycoprotein (HPRG);
(b) rabbit HPRG;
(c) a Tpm-binding, antiangiogenic homologue, variant, domain or fragment of human or rabbit HPRG;
(d) two chain human kininogen human kininogen (HKₐ);
(e) the D5 domain of HKₐ; and
(f) a Tpm-binding, antiangiogenic homologue, variant, domain or fragment of said HKₐ or said D5 domain thereof.

6. The isolated polypeptide, peptide or variant of claim 5 that binds to one or more of SEQ ID NO:21, 22, 23, 24, 25 and 26.

7. A peptide or variant according to claim 1 which is capped at its N-terminus, its C-terminus, or both its N- and its C-terminus.

8. An antibody or an antigen-binding fragment (ABF) thereof which is specific for an epitope of a Tpm isoform expressed on the surface an activated endothelial cell, which antibody or ABF has:
(a) antiangiogenic activity in that it binds to said activated endothelial cell, causing the generation of an antiangiogenic signal in said cell, resulting in
(i) inhibition of migration, invasion, proliferation or angiogenesis, or
(ii) apoptosis; or
(b) proangiogenic activity in that it binds to Tpm on said endothelial cell and inhibits the binding to said cell of a Tpm-binding antiangiogenic agent, thereby permitting or promoting migration, invasion, proliferation or angiogenesis that would otherwise be inhibited by said antiangiogenic agent.

9. An antibody or ABF according to claim 8 which has antiangiogenic activity.

10. An antibody or ABF according to claim 8 which has proangiogenic activity.

11. The antibody or ABF of claim 9, wherein the epitope for which said antibody or ABF is specific is present in, or formed by a polypeptide or peptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20.

12. The antibody or ABF of claim 9 wherein the Tpm-binding antiangiogenic agent is selected from the group consisting of:
(a) human HPRG;
(b) rabbit HPRG;
(c) a Tpm-binding, antiangiogenic homologue, variant, domain or fragment of human or rabbit HPRG;
(d) two chain human kininogen human kininogen (HKₐ);
(e) the D5 domain of HKₐ; and
(f) a Tpm-binding, antiangiogenic homologue, variant, domain or fragment of said HKₐ or said D5 domain thereof.

13. The antibody of claim 8 which is a monoclonal antibody.

14. (canceled)

15. The antibody or ABF of claim 8, which is detectably labeled with a detectable label, which labeled antibody or ABF is useful for detecting a Tpm polypeptide or peptide on endothelial cells.

16. The antibody or ABF of claim 11 which is detectably labeled with a detectable label, which labeled antibody or ABF is useful for detecting a Tpm polypeptide or peptide on endothelial cells.

17. The antibody or ABF of claim 12 which is detectably labeled with a detectable label, which labeled antibody or ABF is useful for detecting a Tpm polypeptide or peptide on endothelial cells.

18. The antibody or ABF of claim 13, which is detectably labeled with a detectable label which labeled antibody or ABF is useful for detecting a Tpm polypeptide or peptide on endothelial cells.

19. (canceled)

20. The antibody of claims 15 wherein the detectable label is a radionuclide, a PET-imageable agent, an MRI-imageable agent, a fluorescer, a fluorogen, a chromophore, a chromogen, a phosphorescer, a chemiluminescer or a bioluminescer.

21. A diagnostically useful Tpm-binding antibody composition comprising:
(a) the detectably labeled antibody or ABF of claim 15; and
(b) a diagnostically acceptable carrier.

22. The composition of claim 21, wherein the detectable label is a radionuclide selected from the group consisting of ³H, ¹⁴C, ³⁵S, ⁶⁷Ga, ⁶⁸Ga, ⁷²As, ⁸⁹Zr, ⁹⁷Ru, ⁹⁹Tc, ¹¹¹In, ¹²³I, ¹²⁵I, ¹³¹I, ¹⁶⁹Yb and ²⁰¹Tl.

23. The composition of claim 21 wherein the detectable label is a fluorescer or fluorogen selected from the group consisting of fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green and Texas Red.

24. The antibody or ABF of claim 9 to which is optionally bound, directly or indirectly, a therapeutically active moiety, which antibody binds to Tpm or to an epitope thereof and inhibits angiogenesis in vitro or in vivo.

25. The antibody or ABF of claim 11 to which is optionally bound, directly or indirectly, a therapeutically active moiety, which antibody binds to Tpm or to an epitope thereof and inhibits angiogenesis in vitro or in vivo.

26. The antibody or ABF of claim 12 to which is optionally bound, directly or indirectly, a therapeutically active moiety, which antibody binds to Tpm or to an epitope thereof and inhibits angiogenesis in vitro or in vivo.

27. The antibody or ABF of claim 13 to which is optionally bound, directly or indirectly, a therapeutically active moiety, which antibody binds to Tpm or to an epitope thereof and inhibits angiogenesis in vitro or in vivo.

28. (canceled)

29. A therapeutic antiangiogenic pharmaceutical composition that inhibits angiogenesis in vitro or in vivo, comprising:
(a) an antiangiogenic effective amount of the antibody or ABF of claim 24; and
(b) a pharmaceutically acceptable carrier.

30. (canceled)

31. The therapeutic pharmaceutical composition of claim 29 wherein the therapeutically active moiety is a radionuclide, drug or toxin.

32. The therapeutic pharmaceutical composition of claim 31, wherein the moiety is a radionuclide is selected from the group consisting of ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb and ²¹⁷Bi.

33. (canceled)

34. The antibody or ABF of claim 10, which binds to Tpm or to an epitope thereof and stimulates angiogenesis in vitro or in vivo.

35. The antibody or ABF of claim 68 which binds to Tpm or to an epitope thereof and stimulates angiogenesis in vitro or in vivo.

36. The antibody or ABF of claim 69 which binds to Tpm or to an epitope thereof and stimulates angiogenesis in vitro or in vivo.

37. The antibody or ABF of claim 13 which binds to Tpm or to an epitope thereof and stimulates angiogenesis in vitro or in vivo.

38. (canceled)

39. A pharmaceutical proangiogenic pharmaceutical composition, comprising:
(a) a proangiogenic effective amount of the antibody or ABF of claim 34; and
(b) a pharmaceutically acceptable carrier.

40. The therapeutic antibody of claim 34 in a form suitable for injection.

41. A cyclic peptide of between about 4 and about 20 amino acids which binds to the D5 domain of HKₐ and inhibit angiogenesis in an in vitro or in vivo assay of angiogenesis.

42. The cyclic peptide of claim 41 selected from the group consisting of:

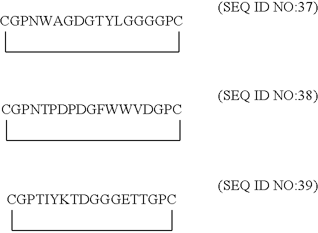

43. A method for inhibiting endothelial cell migration, invasion, proliferation or angiogenesis, or for inducing endothelial cell apoptosis, comprising contacting endothelial cells with an effective amount of a antiangiogenic polypeptide or peptide that binds to Tpm expressed on the surface of activated endothelial cells, and thereby causes said inhibition or said apoptosis.

44. The method of claim 43 wherein the Tpm-binding polypeptide is selected from the group consisting of:
(a) human histidine-proline rich glycoprotein (HPRG);
(b) rabbit HPRG;
(c) a Tpm-binding, antiangiogenic homologue, variant, domain or fragment of human or rabbit HPRG;
(d) two chain human kininogen human kininogen (HKₐ);
(e) the D5 domain of HKₐ;
(f) a Tpm-binding, antiangiogenic homologue, variant, domain or fragment of said HKₐ or said D5 domain thereof;
(g) troponin T;
(h) tropomodulin;
(i) caldesmon;
(j) actin;
(k) calponin;
(l) pEL98;
(m) glutamic dehydrogenase; and
(n) a Tpm-binding, antiangiogenic homologue, variant, domain or fragment of any of (g)-(m).

45. A method for treating a subject having a disease or condition associated with undesired cell migration, invasion, proliferation, or angiogenesis, comprising administering to the subject an effective angiogenesis-inhibiting amount of the a pharmaceutical composition of claim 29.

46-48. (canceled)

49. The method of claim 45 wherein said subject has a tumor, and said angiogenesis inhibition results in reduction in size or growth rate of said tumor or destruction of said tumor.

50. The method of claim 49 wherein said subject is a human.

51. A method for stimulating angiogenesis in a subject in need of enhanced angiogenesis, comprising administering to said subject an effective amount of the pharmaceutical composition of claim 39.

52. A method for detecting in a biological sample the presence of Tpm or an isoform expressed on the surface of activated endothelial cells, comprising the steps of:
(a) contacting the sample with the antibody or ABF of claim 15; and
(b) detecting the presence of the label associated with the sample.

53. A method for detecting the presence of Tpm in a biological sample, comprising the steps of:
(a) contacting the sample with the a detectably labeled antiangiogenic polypeptide or peptide that binds to Tpm expressed on the surface of activated endothelial cells; and
(b) detecting the presence of the label associated with the sample.

54. The method of claim 53 wherein said antiangiogenic polypeptide or peptide is selected from the group consisting of
(a) human histidine-proline rich glycoprotein (HPRG);
(b) rabbit HPRG;
(c) a Tpm-binding, antiangiogenic homologue, variant, domain or fragment of human or rabbit HPRG;
(d) two chain human kininogen human kininogen (HKₐ);
(e) the D5 domain of HKₐ; and
(f) a Tpm-binding, antiangiogenic homologue, variant, domain or fragment of said HKₐ or said D5 domain thereof.

55-59. (canceled)

60. A screening test to identify a test compound as a candidate antiangiogenic molecule that binds to Tpm, comprising
(a) adding the test compound to a mixture of a source of Tpm and a Tpm-binding antiangiogenic polypeptide or peptide agent or anti-Tpm antibody, wherein at least one of (i) said Tpm or (ii) said agent or antibody is detectably labeled;
(b) in parallel, mixing similar amounts of said Tpm and said agent or antibody in the absence of said test compound; and
(c) measuring the binding of said agent with said Tpm in (a) and (b);
wherein, if the binding in (a) is less than the binding in (b), the test is considered positive for said test compound being an inhibitor of said binding,
thereby identifying said test compound as a candidate antiangiogenic molecule.

61. The screening test of claim 60, further comprising testing a test compound that has been identified as a candidate antiangiogenic molecule for its activity as an inhibitor of angiogenesis in an in vitro or in vivo angiogenesis assay.

62. The screening test of claim 60 wherein said agent is selected from the group consisting of:
(a) human histidine-proline rich glycoprotein (HPRG);
(b) rabbit HPRG;
(c) a Tpm-binding, antiangiogenic homologue, variant, domain or fragment of human or rabbit HPRG;
(d) two chain human kininogen human kininogen (HKₐ);
(e) the D5 domain of HKₐ; and
(f) a Tpm-binding, antiangiogenic homologue, variant, domain or fragment of said HKₐ or said D5 domain thereof.

63. An affinity ligand useful for binding to or isolating a Tpm-binding antiangiogenic molecule or cells expressing the binding molecule, comprising the isolated polypeptide or peptide of claim 1, immobilized to a solid support or carrier.

64. (canceled)

65. An affinity ligand useful for binding to or isolating a Tpm-binding antiangiogenic molecule or cells expressing the binding molecule, comprising the isolated polypeptide or peptide of claim 6, immobilized to a solid support or carrier.

66. A method for isolating a Tpm-binding antiangiogenic molecule from a complex mixture comprising:
(a) contacting the mixture with the affinity ligand of claim 63;
(b) allowing any material in the mixture to bind to the ligand;
(c) removing unbound material from the ligand; and
(d) eluting the bound Tpm-binding molecule.

67. The method of claim 66 wherein said anti-angiogenic receptor polypeptide or peptide:
(i) has the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20;
(ii) is a Tpm-binding peptide fragment of one of said sequences; or
(iii) is a Tpm-binding conservative substitution variant of one of said sequences or of said peptide fragment.

68. The antibody or ABF of claim 10, wherein the epitope for which said antibody or ABF is specific is present in, or formed by a polypeptide or peptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ ID NO:20.

69. The antibody or ABF of claim 10 wherein the Tpm-binding antiangiogenic agent is selected from the group consisting of:
(a) human HPRG;
(b) rabbit HPRG;
(c) a Tpm-binding, antiangiogenic homologue, variant, domain or fragment of human or rabbit HPRG;
(d) two chain human kininogen human kininogen (HKa);
(e) the D5 domain of HKₐ; and
(f) a Tpm-binding, antiangiogenic homologue, variant, domain or fragment of said HKₐ or said D5 domain thereof.
